(19) United States
(12) Patent Application Publication
(10) Pub. No.: US 2004/0101874 A1
(43) Pub. Date: May 27, 2004

(54) Targets for therapeutic intervention identified in the mitochondrial proteome

(75) Inventors: Soumitra S. Ghosh, San Diego, CA (US); Eoin D. Fahy, San Diego, CA (US); Bing Zhang, Spring, TX (US); Bradford W. Gibson, Berkeley, CA (US); Steven W. Taylor, San Diego, CA (US); Gary M. Glenn, Encinitas, CA (US); Dale E. Warnock, San Diego, CA (US); Sara P. Gaucher, Castro Valley, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignees: MitoKor Inc., San Diego, CA (US); The Buck Institute for Age Research, Novato, CA (US)

(21) Appl. No.: 10/408,765

(22) Filed: Apr. 4, 2003

Related U.S. Application Data
(60) Provisional application No. 60/412,418, filed on Sep. 20, 2002.
(60) Provisional application No. 60/389,987, filed on Jun. 17, 2002.
(60) Provisional application No. 60/372,843, filed on Apr. 12, 2002.

(51) Int. Cl. C12Q 1/68; C12N 1/00

(52) U.S. Cl. 435/6; 435/317.1

(57) ABSTRACT

Mitochondrial targets for drug screening assays and for therapeutic intervention in the treatment of diseases associated with altered mitochondrial function are provided. Complete amino acid sequences [SEQ ID NOS:1-3025] of polypeptides that comprise the human heart mitochondrial proteome are provided, using fractionated proteins derived from highly purified mitochondrial preparations, to identify previously unrecognized mitochondrial molecular components.

### CROSS-REFERENCES TO RELATED APPLICATIONS

This application claims the benefit of U.S. Provisional Patent Applications No. 60/412,418, filed Sep. 20, 2002; 60/389,987, filed Jun. 17, 2002; and 60/372,843, filed Apr. 12, 2002.

### STATEMENT REGARDING SEQUENCE LISTING SUBMITTED ON CD-ROM

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 465.app.txt which is 14.4 MB and created on Apr. 4, 2003; CD-ROM No.2 is labeled COPY 2, contains the file 465.app.txt which is 14.4 MB and created on Apr. 4, 2003; CD-ROM No. 3 is labeled CRF, contains the file 465.app.txt which is 14.4 MB and created on Apr. 4, 2003.

### BACKGROUND OF THE INVENTION

1. Field of the Invention

The present invention relates generally to compositions and methods for identifying mitochondrial proteins that are useful as targets for therapeutic intervention in treating diseases associated with altered mitochondrial function. More specifically, the invention is directed to proteomic profiling of proteins and polypeptides of mitochondria and to uses of mitochondrial polypeptides in screening assays for, and as targets of, therapeutic agents.

2. Description of the Related Art

Mitochondria are the complex subcellular organelles that manufacture bioenergetically essential adenosine triphosphate (ATP) by oxidative phosphorylation, and that promote direct and indirect biochemical regulation of a wide array of cellular respiratory, oxidative and metabolic processes, including aerobic respiration and intracellular calcium regulation. For example, mitochondria provide the subcellular site for physiologically important processes such as the Krebs cycle, the urea cycle, fatty acid β-oxidation, and heme synthesis. Mitochondria also participate in mechanisms of apoptosis, or programmed cell death (e.g., Newmeyer et al., Cell 79:353-364,1994; Liu et al., Cell 86:147-157, 1996), which is apparently required for, inter alia, normal development of the nervous system and proper functioning of the immune system.

Functional mitochondria contain gene products encoded by mitochondrial genes situated in mitochondrial DNA (mtDNA) and by extramitochondrial (e.g., nuclear) genes not situated in the circular mitochondrial genome. While it has been estimated that a functional human mitochondrion contains on the order of 1,000-1,500 distinct proteins (Lopez et al., 2000 Electrophoresis 21:3427; Scheffler, I. E., Mitochondria, 1999 Wiley-Liss, Inc., New York; Rabilloud et al., 1998 Electrophoresis 19:1006; Scheffleretal., 2001 Mitochondrion 1:161; Schatz, G., 1995 Biochem. Biophys. Acta Mol. Basis Dis. 1271:123), the 16.5 kb mtDNA encodes 22 tRNAs, two ribosomal RNAs (12s and 16s rRNA) and only 13 polypeptides, which are enzymes of the electron transport chain (ETC), the elaborate multi-subunit complex mitochondrial assembly where, for example, respiratory oxidative phosphorylation takes place. (See, e.g., Wallace et al., in Mitochondria & Free Radicals in Neurodegenerative Diseases, M. F. Beal, N. Howell and I. BodisWollner, eds., 1997 Wiley-Liss, Inc., New York, pp. 283-307, and references cited therein; see also, e.g., Scheffler, I. E., Mitochondria, 1999Wiley-Liss, Inc., New York.) Mitochondrial DNA thus includes gene sequences encoding seven subunits of NADH dehydrogenase, also known as ETC Complex I (ND1, ND2, ND3, ND4, ND4L, ND5 and ND6); one subunit of ETC Complex III (ubiquinol: cytochrome c oxidoreductase, Cytb); three cytochrome c oxidase (ETC Complex IV) subunits (COX1, COX2 and COX3); and two proton-translocating ATP synthase (Complex V) subunits (ATPase6 and ATPase8). All other mitochondrial constituent polypeptides are presumed to be encoded by genes of the extramitochondrial genome, and the number and identities of a large number of these polypeptides remain unknown. Accordingly, for most of the estimated 25,000-40,000 proteins encoded by the human nuclear genome (Venter et al., 2001 Science 291:1304; Lander et al., 2001 Nature 409:860) little is known regarding subcellular localization, for example, which proteins may be molecular components of mitochondria.

Mitochondria contain an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix (for review, see, e.g., Ernster et al., 1981 J. Cell Biol. 91:227s.) The cristae, originally postulated to occur as infoldings of the inner mitochondrial membrane, have recently been characterized using three-dimensional electron tomography as also including tube-like conduits that may form networks, and that can be connected to the inner membrane by open, circular (30 nm diameter) junctions (Perkins et al., 1997, JI. of Struct. Biol. 119:260). While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (greater than about 10 kD) molecules.

Four of the five multisubunit protein complexes (Complexes I, III, IV and V) that mediate ETC activity are localized to the inner mitochondrial membrane. The remaining ETC complex (Complex II) is situated in the matrix. In at least three distinct chemical reactions known to take place within the ETC, protons are moved from the mitochondrial matrix, across the inner membrane, to the intermembrane space. This disequilibrium of charged species creates an electrochemical membrane potential of approximately 220 mV referred to as the “protonmotive force” (PMF). The PMF, which is often represented by the notation Δp, corresponds to the sum of the electric potential (Δψm) and the pH differential (ΔpH) across the inner membrane according to the equation

Δp=Δωm−ZΔpH

wherein Z stands for −2.303 RT/F. The value of Z is −59 at 25° C. when Δp and Δψm are expressed in mV and ΔpH is expressed in pH units (see, e.g., Ernster et al., J. Cell Biol. 91:227s, 1981 and references cited therein).

Δψm provides the energy for phosphorylation of adenosine diphosphate (ADP) to yield ATP by ETC Complex V, a process that is coupled stoichiometrically with transport of a proton into the matrix. Δψm is also the driving force for the influx of cytosolic Ca²⁺ into the mitochondrion. Under normal metabolic conditions, the inner membrane is impermeable to proton movement from the intermembrane space into the matrix, leaving ETC Complex V as the sole means whereby protons can return to the matrix. When, however, the integrity of the inner mitochondrial membrane is compromised, as occurs during mitochondrial permeability transition (MPT) that accompanies certain diseases associated with altered mitochondrial function, protons are able to bypass the conduit of Complex V without generating ATP, thereby uncoupling respiration. During MPT, Δψm collapses and mitochondrial membranes lose the ability to selectively regulate permeability to solutes both small (e.g., ionic Ca²⁺, Na⁺, K⁺ and H⁺) and large (e.g., proteins).

A number of diseases, disorders or conditions, including degenerative diseases, are thought to be caused by, or are associated with, alterations in mitochondrial function as provided herein. These disorders include Alzheimer's Disease (AD), diabetes mellitus, Parkinson's Disease (PD), Huntington's disease, Freidreich's ataxia, atherosclerosis, hypertension, ischemia-reperfusion injury, osteoarthritis, inflammatory diseases, amyotrophic lateral sclerosis (ALS), Wilson disease, autosomal recessive hereditary spastic paraplegia, Leigh syndrome, benign and fatal infantile myopathies, multiple sclerosis, dystonia, Leber's hereditary optic neuropathy, schizophrenia, cancer; psoriasis; Down's syndrome, hyperproliferative disorders; mitochondrial diabetes and deafness (MIDD) and myodegenerative disorders such as “mitochondrial encephalopathy, lactic acidosis, and stroke” (MELAS), and “myoclonic epilepsy ragged red fiber syndrome” (MERRF), as well as other mitochondrial respiratory chain diseases (reviewed in Chinnery et al., 1999 J. Med. Genet. 36:425; see also references cited therein). Diseases associated with altered mitochondrial function thus include these and other diseases in which one or more levels of an indicator of altered mitochondrial function differ in a statistically significant manner from the corresponding indicator levels found in clinically normal subjects known to be free of a presence or risk of such disease. Other diseases involving altered metabolism or respiration within cells may also be regarded as diseases associated with altered mitochondrial function, for example, those in which free radicals such as reactive oxygen species (ROS) contribute to pathogenesis. Certain diseases associated with altered mitochondrial function appear to involve states of insufficient apoptosis (e.g., cancer and autoimmune diseases) or excessive levels of apoptosis (e.g., stroke and neurodegeneration). For a general review of apoptosis, and the role of mitochondria therein, see, e.g., Green and Reed, Science 281:1309-1312, 1998; Green, Cell 94:695-698, 1998 and Kromer, Nature Medicine 3:614-620, 1997. The extensive list of additional diseases associated with altered mitochondrial function continues to expand as aberrant mitochondrial or mitonuclear activities are implicated in particular disease processes.

For instance, free radical production in biological systems is known to result in the generation of reactive species that can chemically modify molecular components of cells and tissues. Such modifications can alter or disrupt structural and/or functional properties of these molecules, leading to compromised cellular activity and tissue damage. Mitochondria are a primary source of free radicals in biological systems (see, e.g., Murphy et al., 1998 in Mitochondria and Free Radicals in Neurodegenerative Diseases, Beal, Howell and Bodis-Woliner, Eds., Wiley-Liss, New York, pp. 159-186 and references cited therein), and altered mitochondrial function, such as failure at any step of the mitochondrial electron transport chain (ETC), may also lead to the generation of highly reactive free radicals. Thus, free radicals generated in biological systems, including free radicals resulting from altered mitochondrial function or from extramitochondrial sources, include reactive oxygen species (ROS), for example, superoxide, peroxynitrite and hydroxyl radicals, and potentially other reactive species that may be toxic to cells. Diseases associated with altered mitochondrial function therefore include disorders in which free radicals contribute to pathogenesis at the molecular level (see, e.g., Halliwell B. and J. M. C. Gutteridge, Free Radicals in Biology and Medicine, 1989 Clarendon Press, Oxford, UK).

A particularly prevalent example of a disease associated with altered mitochondrial function is type 2 diabetes mellitus, or “late onset” diabetes, a common, degenerative disease affecting 5 to 10 percent of the population in developed countries. The propensity for developing type 2 diabetes mellitus (“type 2 DM”) is reportedly maternally inherited, suggesting a mitochondrial genetic involvement. (Alcolado, J. C. and Alcolado, R., Br. Med. J. 302:1178-1180 (1991); Reny, S. L., International J. Epidem. 23:886-890 (1994)). Diabetes is a heterogeneous disorder with a strong genetic component; monozygotic twins are highly concordant and there is a high incidence of the disease among first degree relatives of affected individuals.

At the cellular level, the degenerative phenotype that may be characteristic of late onset diabetes mellitus includes indicators of altered mitochondrial respiratory function, for example impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, ciruculating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

Despite intense effort, nuclear genes that segregate with diabetes mellitus are rare and include, for example, mutations in the insulin gene, the insulin receptor gene and the glucokinase gene. By comparison, although a number of altered mitochondrial genes that segregate with diabetes mellitus have been reported (see generally e.g., PCT/US95/04063), relationships amongst mitochondrial and extramitochondrial factors that contribute to cellular respiratory and/or metabolic activities as they pertain to diabetes remain poorly understood.

Current pharmacological therapies for type 2 DM include injected insulin, and oral agents that are designed to lower blood glucose levels. Currently available oral agents include (i) the sulfonylureas, which act by enhancing the sensitivity of the pancreatic beta cell to glucose, thereby increasing insulin secretion in response to a given glucose load; (ii) the biguanides, which improve glucose disposal rates and inhibit hepatic glucose output; (iii) the thiazolidinediones, which improve peripheral insulin sensitivity through interaction with nuclear peroxisome proliferator-activated receptors (PPAR, see, e.g., Spiegelman, 1998 Diabetes 47:507-514; Schoonjans et al., 1997 Curr. Opin. Lipidol. 8:159-166; Staels et al., 1997 Biochimie 79:95-99), (iv) repaglinide, which enhances insulin secretion through interaction with ATP-dependent potassium channels; and (v) acarbose, which decreases intestinal absorption of carbohydrates. It is clear that none of the current pharmacological therapies corrects the underlying biochemical defect in type 2 DM. Neither do any of these. currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

Clearly there is a need for improved diagnostic methods for early detection of a risk for developing a disease associated with altered mitochondrial function, and for better therapeutics that are specifically targeted to correct biochemical and/or metabolic defects responsible for such disease, regardless of whether such a defect underlying altered mitochondrial function may have mitochondrial or extramitochondrial origins. The present invention provides compositions and methods related to identification of mitochondrial targets for therapeutic intervention in treating these diseases, and offers other related advantages.

### BRIEF SUMMARY OF THE INVENTION

The present invention provides the identities of 3025 polypeptide sequences [SEQ ID NOS:1-3025] that are constituents of the human mitochondrial proteome. It is therefore an aspect of the present invention to provide a method for identifying a mitochondrial target for therapeutic intervention in treatment of a disease associated with altered mitochondrial function, comprising (a) determining a presence, in a biological sample from a subject known to have or suspected of having a disease associated with altered mitochondrial function, of at least one modified polypeptide, the modified polypeptide comprising at least one modification to a polypeptide having an amino acid sequence as set forth in any one of SEQ ID NOS 1-3025; and (b) correlating the modification with at least one disease associated with altered mitochondrial function, and therefrom identifying a mitochondrial target for therapeutic intervention.

In certain embodiments the modified polypeptide exhibits altered biological activity. In certain embodiments the biological sample is selected from the group consisting of blood, skin, skeletal muscle, liver and cartilage. In certain embodiments the disease associated with altered mitochondrial function is Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF) or cancer. In certain embodiments the modification is an amino acid substitution, an amino acid insertion, an amino acid deletion, a posttranslational modification or an altered expression level, and in certain further embodiments the posttranslational modification is glycosylation, phosphorylation, nitration, nitrosylation, amidation, fatty acylation or oxidative modification, including, for example, oxidative post-translational modification of tryptophan residues.

In certain other embodiments the present invention provides a method of identifying an agent for treating a disease associated with altered mitochondrial function, comprising (a) contacting a candidate agent with a biological sample from a subject having a disease associated with altered mitochondrial function, wherein the sample comprises at least one polypeptide that exhibits altered biological activity which accompanies the disease and wherein the polypeptide is (i) a polypeptide having an amino acid sequence as set forth in any one of SEQ ID NOS 1-3025, or (ii) a modified polypeptide that comprises at least one modification to a polypeptide having an amino acid sequence as set forth in any one of SEQ ID NOS 1-3025; and (b) determining an increase or decrease in the altered biological activity of the polypeptide in the presence of the candidate agent relative to the level of the altered biological activity in the absence of the candidate agent, and therefrom identifying an agent for treating a disease associated with altered mitochondrial function.

In certain embodiments the altered biological activity is an indicator of altered mitochondrial function that is ATP biosynthesis (e.g., an ATP biosynthesis factor), oxidative phosphorylation, mitochondrial calcium uptake, mitochondrial calcium release, maintenance of inner mitochondrial membrane potential, mitochondrial permeability transition, ETC-mediated electron transport or mitochondrial intermembrane space protein release. In certain other embodiments the sample is a cell, a mitochondria enriched sample, an isolated mitochondrion or a submitochondrial particle. In certain embodiments the disease associated with-altered mitochondrial function is Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF) or cancer.

According to certain other embodiments there is provided by the present invention a method of treating a disease associated with altered mitochondrial function comprising administering to a subject in need thereof an agent that compensates for at least one biological activity of a polypeptide that exhibits altered biological activity which accompanies the disease, wherein the polypeptide is (i) a polypeptide having an amino acid sequence as set forth in any one of SEQ ID NOS 1-3025, or (ii) a modified polypeptide that comprises at least one modification to a polypeptide having an amino acid sequence as set forth in any one of SEQ ID NOS 1-3025. In another embodiment the invention provides a method for identifying a risk for having or a presence of a disease associated with altered mitochondrial function, comprising (a) determining a presence, in a biological sample from a subject suspected of having a disease associated with altered mitochondrial function, of at least one modified polypeptide, the modified polypeptide comprising at least one modification to a polypeptide having an amino acid sequence as set forth in any one of SEQ ID NOS 1-3025, wherein the modification correlates with at least one disease associated with altered mitochondrial function, and therefrom identifying a risk for or presence of disease.

Certain other embodiments of the invention provide a method of identifying an agent for treating a disease associated with altered mitochondrial function, comprising (a) contacting a candidate agent with an isolated polypeptide that exhibits altered biological activity which accompanies a disease associated with altered mitochondrial function, wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025; and (b) determining an increase or decrease in the altered biological activity of the polypeptide in the presence of the candidate agent relative to the level of the altered biological activity in the absence of the candidate agent, and therefrom identifying an agent for treating a disease associated with altered mitochondrial function. In certain further embodiments the disease associated with altered mitochondrial function is Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF), or cancer. In other further embodiments the isolated polypeptide is present in a preparation that is a submitochondrial particle, a proteoliposome or a mitochondrial protein fraction.

In another embodiment the invention provides a method of identifying an agent for treating a disease associated with altered mitochondrial function, comprising (a) administering a candidate agent to a subject having a disease associated with altered mitochondrial function; and (b) determining, in a first biological sample obtained from the subject prior to the step of administering the candidate agent and in a second biological sample obtained from the subject subsequent to the step of administering the candidate agent, wherein each of said first and second samples comprises at least one polypeptide that exhibits altered biological activity which accompanies said disease and wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025, an increase or decrease in the altered biological activity of the polypeptide in the second sample relative to the level of the altered biological activity in the first sample, and therefrom identifying an agent for treating a disease associated with altered mitochondrial function. In a further embodiment, the altered biological activity is an indicator of altered mitochondrial function that is ATP biosynthesis, oxidative phosphorylation, calcium uptake, calcium release, maintenance of inner mitochondrial membrane potential, mitochondrial permeability transition, ETC-mediated electron transport or intermembrane space protein release. In another further embodiment the sample is a cell, a mitochondria enriched sample, an isolated mitochondrion or a submitochondrial particle. In certain other further embodiments, the disease associated with altered mitochondrial function is Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF), or cancer.

These and other aspects of the present invention will become evident upon reference to the following detailed description and attached drawings. In addition, various references are set forth below which describe in more detail certain procedures or compositions and are therefore incorporated by reference in their entireties.

### BRIEF DESCRIPTION OF THE DRAWINGS

FIG. 1 shows representative western immunoblot analysis (FIG. 1A) of indicated mitochondrial ETC proteins in sucrose density gradient fractionated isolated human heart mitochondria, following resolution of proteins by one-dimensional polyacrylamide gel electrophoresis (FIG. 1B).

FIG. 2 shows a representative MALDI mass spectrum for a single band excised from a one-dimensional polyacrylamide gel following electrophoretic resolution of proteins from sucrose density gradient fractionated isolated human heart mitochondria. Peptides are from indicated mitochondrial proteins as follows: β=ATP synthase beta subunit, γ=ATP synthase gamma subunit, eCoA=enlyl-CoA hydratase, and vd=voltage dependent anion channel 1 (VDAC-1). (K=keratin.)

FIG. 3 shows products of tryptophan oxidation in proteins.

FIG. 4 shows MALDI-TOF mass spectrometry of two peptides from complex I subunit NDUFS4 displaying (A) tryptophan and (B) methionine oxidation. The samples were as follows (i) human heart mitochondria complex I (HHM individual #1) prepared by sucrose density gradient fractionation (SDG) and 1D electrophoresis; (ii) HHM individual #1 prepared by immunocapture and ID electrophoresis (iii) HHM individual #2 prepared by immunocapture and 1D electrophoresis; (iv) HHM individuals #3,4,5 (pooled) prepared by SDG and 1D electrophoresis; (v) bovine heart mitochondria (BHM animal #1) prepared by SDG and 1D electrophoresis; (vi) (BHM animal #2) prepared by SDG and 2D electrophoresis.

FIG. 5 shows a comparison of the distribution of (a) tryptophan and (b) methionine oxidation for complex I subunit peptides.

### DETAILED DESCRIPTION OF THE INVENTION

The present invention provides a method for identifying mitochondrial polypeptide targets for therapeutic intervention in the treatment of diseases associated with altered mitochondrial function, and a method for identifying agents for treating such diseases, as well as other related advantages.

The invention derives from characterization of the human heart mitochondrial proteome as described herein, to arrive at the surprising discovery and recognition for the first time that polypeptides having the amino acid sequences set forth in SEQ ID NOS:1-3025 are mitochondrial molecular components. This unexpected determination, that isolated human mitochondria comprise polypeptides having the amino acid sequences set forth in SEQ ID NOS:1-3025, is usefully combined with methods for determining the presence of a disease associated with altered mitochondrial function, and with methods for determining modification to, and altered biological activity of, a polypeptide, to provide targets for drug-screening assays and for therapeutic agents. According to certain embodiments, the invention relates to determination of at least one modified polypeptide that comprises a modification to a polypeptide having an amino acid sequence as set forth in any one of SEQ ID NOS:1-3025, and according to certain other embodiments the invention relates to determination of a profile comprising a plurality (e.g., two or more) of polypeptides having distinct amino acid sequences wherein at least one such polypeptide has one of the amino sequences set forth in SEQ ID NOS:1-3025, and has not been previously identified as a mitochondrial component.

Thus, it is an aspect of the present invention to provide a method for identifying a mitochondrial target for therapeutic intervention in treatment of a disease associated with altered mitochondrial function, comprising (a) determining a presence, in a biological sample from a subject known to have or suspected of having a disease associated with altered mitochondrial function, of at least one modified polypeptide, the modified polypeptide comprising at least one modification to a polypeptide having an amino acid sequence as set forth in any one of SEQ ID NOS 1-3025; and (b) correlating the modification with at least one disease associated with altered mitochondrial function, and therefrom identifying a mitochondrial target for therapeutic intervention.

Biological samples may comprise any tissue or cell preparation containing mitochondria. Biological samples may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortal, immortalized or immortalizable cell lines (e.g., capable of at least ten cell doublings in vitro), somatic cell hybrid or cytoplasmic hybrid “cybrid” cell lines (including mitochondrial cybrid cells having nuclear and mitochondrial DNAs of differing biological origins, see, e.g., U.S. Pat. No. 5,888,498 and International Publication No. WO 95/26793), differentiated or differentiatable cell lines, transformed cell lines and the like. In certain preferred embodiments of the invention, the subject or biological source may be suspected of having or being at risk for having a disease associated with altered mitochondrial function, including, for example, altered mitochondrial molecular composition or constitution, or oxidative modification of one or more mitochondrial proteins, and in certain preferred embodiments of the invention the subject or biological source may be known to be free of a risk or presence of such a disease. In certain other preferred embodiments a biological sample comprises a cybrid cell line having nuclear and mitochondrial DNAs of differing biological origins, which in certain embodiments may be a human cell, an immortal cell, a neuronal cell, a neuroblastoma or other transformed cell, for example, a SH-SY5Y human neuroblastoma cell. In certain other particularly preferred embodiments a biological sample comprises a sample readily obtained from a subject or biological source, such as blood, skin, skeletal muscle, liver or cartilage.

By way of background, mitochondria are comprised of “mitochondrial molecular components”, which may be any protein, polypeptide, peptide, amino acid, or derivative thereof; any lipid, fatty acid or the like, or derivative thereof; any carbohydrate, saccharide or the like or derivative thereof, any nucleic acid, nucleotide, nucleoside, purine, pyrimidine or related molecule, or derivative thereof, or the like; or any other biological molecule that is a constituent of a mitochondrion, which may include molecules that are integral or stable components of mitochondrial structure, and may also include molecules that may transiently associate with mitochondria under certain conditions, for example, regulated intracellular events that involve mitochondria. In the most preferred embodiments, the present invention is directed to compositions and methods that relate to those mitochondrial molecular components that are mitochondrial polypeptides or proteins, although the invention need not be so limited.

In certain preferred embodiments of the present invention, a mitochondrial protein fraction is derived from the biological sample as provided herein. A protein fraction may be any preparation that contains at least one protein that is present in the sample and which may be obtained by processing a biological sample according to any biological and/or biochemical methods useful for isolating or otherwise separating a protein from its biological source. Those familiar with the art will be able to select an appropriate method depending on the biological starting material and other factors. Such methods may include, but need not be limited to, cell fractionation, density sedimentation, differential extraction, salt precipitation, ultrafiltration, gel filtration, ion-exchange chromatography, partition chromatography, hydrophobic chromatography, reversed-phase chromatography, one- and two-dimensional electrophoresis, affinity techniques or any other suitable separation method.

It will be noted that in certain particularly preferred embodiments of the present invention, at least one sample as described herein comprises a “mitochondria enriched” sample, which refers to a sample that comprises one or more mitochondria and that is substantially depleted (i.e., partially or fully depleted, where the degree of depletion of a given component can be quantified to show that its presence has been reduced in a statistically significant manner) of one or more non-mitochondrial marker proteins to the extent such markers can be removed from a preparation and are detectable, as described herein and known to the art. Thus, for example, cell fractionation techniques for the enrichment and detection of mitochondria, and/or biochemical markers characteristic of these and other defined organelles, may be used to determine that a particular subcellular fraction containing one or more detectable organelle-specific or organelle-associated markers or polypeptides, as provided herein, is substantially enriched in mitochondria (see, e.g., Ernster et al., 1981 J. Cell Biol. 91:227s; see also, e.g., Rickwood et al., 1987, Mitochondria, a practical approach (Darley-Usmar, R., Wilson,, Ed.), IRL Press; Storrie and Madden, 1990 Methods in Enzymology 182, 203-225).

For example, and in certain preferred embodiments including methods for determining the presence in a biological sample of a mitochondrial target polypeptide for therapeutic intervention-or for screening a candidate agent for its ability to alter the biological activity of such a target, a mitochondrial molecular component such as any protein or polypeptide having an amino acid sequence as set forth in any one of SEQ ID NOS:1-3025 may be obtained from a preparation of isolated mitochondria and/or from a preparation of isolated submitochondrial particles (SMP). Techniques for isolating mitochondria and for preparing SMP are well known to the person having ordinary skill in the art and may include certain minor modifications as appropriate for the particular conditions selected (e.g., Smith, A. L., Meths. Enzymol. 10:81-86; Darley-Usman et al., (eds.), Mitochondria: A Practical Approach, IRL Press, Oxford, UK; Storrie et al., 1990 Meths. Enzymol. 182:203-255). Cell or tissue lysates, homogenates, extracts, suspensions, fractions or the like, or other preparations containing partially or fully purified mitochondrial molecular components such as mitochondrial proteins (e.g., MCA) may also be useful in these and related embodiments. According to certain other related embodiments, one or more isolated mitochondrial molecular components such as isolated targets for therapeutic intervention in the treatment of a disease associated with altered mitochondrial function may be present in membrane vesicles such as uni- or multilamellar membrane vesicles, or reconstituted into naturally derived or synthetic liposomes or proteoliposomes or similar membrane-bounded compartments, or the like, according to generally accepted methodologies (e.g., Jezek et al., 1990 J. Biol. Chem. 265:10522-10526).

Affinity techniques are particularly useful-in the context of the present invention, and may include any method that exploits a specific binding interaction with a mitochondrial protein or peptide to effect a separation. Other useful affinity techniques include immunological techniques for isolating specific proteins or peptides, which techniques rely on specific binding interaction between antibody combining sites for antigen and antigenic determinants present in the proteins or peptides. Immunological techniques include, but need not be limited to, immunoaffinity chromatography, immunoprecipitation, solid phase immunoadsorption or other immunoaffinity methods. See, for example, Scopes, R. K., Protein Purification: Principles and Practice, 1987, Springer-Verlag, NY; Weir, D. M., Handbook of Experimental Immunology, 1986, Blackwell Scientific, Boston; Deutscher, M. P., Guide to Protein Purification, 1990, Methods in Enzymology Vol. 182, Academic Press, New York; and Hermanson, G. T. et al., Immobilized Affinity Ligand Techniques, 1992, Academic Press, Inc., California; which are hereby incorporated by reference in their entireties, for details regarding techniques for isolating and characterizing proteins and peptides, including affinity techniques.

The term “isolated” means that the material is removed from its original environment (e.g., the natural environment if it is naturally occurring). For instance, a naturally occurring protein or peptide present in a living animal is not isolated, but the same protein or peptide, separated from some or all of the co-existing materials in the natural system, is isolated. Thus, for example, such proteins could be part of a multisubunit complex or a membrane vesicle, and/or such peptides could be part of a composition, and still be isolated in that such complex, vesicle or composition is not part of its natural environment.

“Biological activity” of a protein may be any detectable parameter that directly relates to a condition, process, pathway, dynamic structure, state or other activity involving the protein and that permits detection of altered protein function in a biological sample from a subject or biological source, or in a preparation of the protein isolated therefrom. The methods of the present invention thus pertain in part to such correlation where the protein having biological activity may be, for example, an enzyme, a structural protein, a receptor, a ligand, a membrane channel, a regulatory protein, a subunit, a complex component, a chaperone protein, a binding protein or a protein having a biological activity according to other criteria including those provided herein. Such activity may include the amount of a protein that is present, or the amount of a given protein's function that is detectable.

“Altered biological activity” of a protein may refer to any condition or state, including those that accompany a disease associated with altered mitochondrial function, for example, a disease or disorder characterized by altered (e.g., increased or decreased in a statistically significant manner relative to an appropriate control) mitochondrial molecular composition or constitution or by modification of a mitochondrial protein as provided herein (and in particular, e.g., a modification to a polypeptide that in its unmodified form comprises an amino acid sequence as set forth in any one of SEQ ID NOS:1-3025), where any structure or activity that is directly or indirectly related to a particular protein's function (or multiple functions) has been changed in a statistically significant manner relative to a control or standard.

Altered biological activity may have its origin in deletion, substitution or insertion of one or more amino acids in a mitochondrial protein; in posttranslational modification of a mitochondrial protein; in an altered expression level (e.g., a statistically significant increase or decrease in the amount present) of a mitochondrial protein; in oxidatively modified structures or oxidative events as well as in oxidation-independent structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like. According to certain embodiments as provided herein, altered biological activity of a protein may also result from direct or indirect interaction of a biologically active protein with an introduced agent such as an agent for treating a disease associated with altered mitochondrial function as described herein, for example, a small molecule.

Additionally, altered biological activity of a mitochondrial protein (including proteins having any amino acid sequence set forth in SEQ ID NOS:1-3025 or modified forms of such proteins as provided herein) may result in altered respiratory, metabolic or other biochemical or biophysical activity in some or all cells of a biological source having a disease associated with altered mitochondrial function. As non-limiting examples, markedly impaired ETC activity may be related to altered biological activity of at least one protein, as may be generation of increased free radicals such as reactive oxygen species (ROS) or defective oxidative phosphorylation. As further examples, altered mitochondrial membrane potential, induction of apoptotic pathways and formation of a typical chemical and biochemical crosslinked species within a cell, whether by enzymatic or non-enzymatic mechanisms, may all be regarded as indicative of altered protein biological activity. Non-limiting examples of altered protein biological activity are described in greater detail below.

Thus, by way of non-limiting examples, coordinated replication of nuclear and mitochondrial DNA (reviewed in Clayton, D. A., 1992, Int. Rev. Cytol. 141, 217-232; and Shadel and Clayton, 1997, Annu. Rev. Biochem. 66, 409-435), or mitochondrial DNA transcription and RNA processing (Shadel and Clayton, 1996, Methods Enzymol. 264,149-158; Micol et al., 1996, Methods Enzymol. 264,158-173) both incompletely understood processes involving a large number of mitochondrial and extramitochondrial proteins, may be altered mitochondrial functions in certain diseases associated with altered mitochondrial function as provided herein. According to these examples, the disclosure herein—that polypeptides such as those listed in Table 2 alongside the functional classifications such as “carrier”, “DNA synthesis”, “nucleotide metabolism”, “transcription” and “transport”, are mitochondrial components—provides targets for therapeutic intervention in such diseases. In like manner, the disclosure herein that other polypeptides having amino acid sequences as set forth in SEQ ID NOS:1-3025 are mitochondrial components also identifies these proteins as targets for therapeutic intervention in a disease associated with altered mitochondrial function. Moreover, functional classifications of these proteins as recited in Tables 1 and 2 and in the GenBank annotations cited therein (which are incorporated by-reference) provides further guidance to those familiar with the art regarding how readily and without undue experimentation to select a biological activity for interrogation, to determine whether such activity is altered in a sample according to art accepted methodologies.

According to certain embodiments of the invention, a mitochondrial polypeptide is isolated from a biological sample following exposure of the sample to a “biological stimulus”, which may include any naturally occurring or artificial (including recombinant) compound that is capable of inducing altered biological activity of a mitochondrial molecular component which is, in preferred embodiments, a mitochondrial polypeptide. Thus, a biological stimulus may be employed, according to certain of the subject invention methods, to effect a perturbation of the biological status of a cell in a manner that alters biological activity of a mitochondrial polypeptide, such that the altered activity can be detected using any methodology described or referred to herein or known to the art, for example, according to the mass spectrometric fingerprinting methods described herein and in the cited references. Non-limiting examples of biological stimuli include antibodies, hormones, cytokines, chemokines, biologically active polypeptides and peptides and other soluble mediators, apoptogens, signal transduction agents, small molecules, cations and ionophores, physical and chemical stressors, and the like.

The polypeptides of the present invention are preferably provided in an isolated form, and in certain preferred embodiments are purified to homogeneity. The terms “fragment,” “derivative” and “analog” when referring to mitochondrial proteins such as polypeptides identified herein as mitochondrial components and having amino acid sequences as set forth in at least one of SEQ ID NOS:1-3025, or when referring to modified polypeptides that comprise at least one modification to a polypeptide having an amino acid sequence as set forth in any one of SEQ ID NOS:1-3025 as provided herein, refers to any polypeptide or protein that retains essentially the same biological function or activity as such polypeptide. Thus, an analog includes a proprotein which can be activated by cleavage of the proprotein portion to produce an active polypeptide.

The polypeptide (e.g., a human mitochondrial protein or polypeptide having an amino acid sequence set forth in SEQ ID NOS:1-3025) of the present invention may be a naturally occurring, a recombinant polypeptide or a synthetic polypeptide, and is preferably an isolated, naturally occurring polypeptide. Modified polypeptides according to the present invention comprise at least one modification (e.g., a structural change that occurs with statistical significance in a disease associated with altered mitochondrial function) to a protein or polypeptide having an amino acid sequence as set forth in any one of SEQ ID NOS:1-3025. The protein or polypeptide may therefore be an unmodified polypeptide or may be a polypeptide that has been posttranslationally modified, for example by glycosylation (e.g., N-linked glycosylation via asparagines residues, or O-linked glycoslyation via serine or threonine residues or post-biosynthetic glycation, etc.), phosphorylation, oxidation or oxidative modification, nitration, nitrosylation, amidation, fatty acylation including glycosylphosphatidylinositol anchor modification or the like, phospholipase cleavage such as phosphatidylinositol-specific phospholipase c mediated hydrolysis or the like, protease cleavage, dephosphorylation or any other type of protein posttranslational modification such as a modification involving formation or cleavage of a covalent chemical bond, although the invention need not be so limited and also contemplates non-covalent associations of proteins with other biomolecules (e.g., lipoproteins, metalloproteins, etc.). Methods for determining the presence of such modifications are well known in the art (e.g., Scopes, R. K., Protein Purification: Principles and Practice, 1987, Springer-Verlag, NY; Angeletti, Ed., Techniques in Protein Chemistry III, Academic Press, Inc., New York, 1993; Baynes et al., 1991 Diabetes 40:405; Baynes et al., 1999 Diabetes 48:1; Yamakura et al., 1998 J. Biol. Chem. 273:14085; MacMillan et al., 1998 Biochem. 37:1613; see also PCT/US01/14066).

A fragment, derivative or analog of a mitochondrial molecular component polypeptide or protein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, which may include a posttranslational modification or an adduct (e.g., an oxidative adduct), or (iii) one in which one or more of the amino acid residues are deleted, or (iv) one in which additional amino acids are fused to the polypeptide, including a signal sequence, a leader sequence or a proprotein sequence or the like, and also including additional peptide or non-peptide moieties that may be added to proteins such as ubiquitin, glutathione, thioredoxin and the like. Such fragments, derivatives and analogs are deemed to be within the scope of those skilled in the art from the teachings herein.

The polypeptides of the present invention include mitochondrial polypeptides and proteins having amino acid sequences that are identical or similar to sequences known in the art. As known in the art “similarity” between two polypeptides is determined by comparing the amino acid sequence and conserved amino acid substitutes thereto of the polypeptide to the sequence of a second polypeptide. Fragments or portions of the polypeptides of the present invention may be employed for producing the corresponding full-length polypeptide by peptide synthesis; therefore, the fragments may be employed as intermediates for producing the full-length polypeptides.

As described herein, isolation of a mitochondrial polypeptide component such as a mitochondrial molecular component with which an agent identified according to the methods of the invention interacts refers to physical separation of such a complex from its biological source, and may be accomplished by any of a number of well known techniques including but not limited to those described herein, and in the cited references. Without wishing to be bound by theory, a compound that “binds a mitochondrial component” can be any discrete molecule, agent compound, composition of matter or the like that may, but need not, directly bind to a mitochondrial molecular component, and may in the alternative bind indirectly to a mitochondrial molecular component by interacting with one or more additional components that bind to a mitochondrial molecular component. These or other mechanisms by which a compound may bind to and/or associate with a mitochondrial molecular component are within the scope of the claimed methods. Binding to a mitochondrial component may under certain conditions result in altered biological activity of the mitochondrial component.

According to certain preferred embodiments of the present invention, proteins and polypeptides comprising one or more of the amino acid sequences set forth in SEQ ID NOS:1-3025, which include polypeptides not previously known to be mitochondrial components, may be targets for drug screening and/or for therapeutic intervention. A “target” refers to a biochemical entity involved in a biological process, typically a protein that plays a useful role in the physiology or biology of a subject or biological source. A therapeutic composition or compound may bind to, alter the conformation of, impair or enhance the activity of or otherwise influence a target to alter (e.g., increase or decrease in a statistically significant manner relative to an appropriate untreated control) its function. As used herein, targets can include, but need not be limited to, proteins having a mitochondrial function classification as summarized in Table 2 and as described in greater detail below.

For example, targets may include proteins that are components of, or that associate with, mitochondrial ETC complexes, Krebs cycle or TCA cycle components including any molecules functionally linked (e.g., as substrates, cofactors, intermediates, biochemical donor or acceptor species, or the like) to such components, transport protein or carrier protein assemblies, factors or complexes involved in DNA (including mtDNA) replication or transcription or in translation of mRNA, cellular receptors, G-proteins or G-protein coupled receptors, kinases, phosphatases, ion channels, lipases, phosholipases, nuclear receptors and factors, intracellular structures, components of signal transduction and apoptotic pathways, and the like.

Methods for identifying a mitochondrial target (e.g., a pharmaceutical target such as a target for therapeutic intervention in a disease associated with altered mitochondrial function as provided herein, for instance, diabetes mellitus, a neurodegenerative disease, a disease associated with inappropriate cell proliferation or cell survival, or a cardiovascular condition) include providing a compound that modulates expression level, structure and/or activity of a particular mitochondrial protein (e.g., a component of the human mitochondrial proteome such as any one or more of the proteins having amino acid sequences set forth in SEQ ID NOS:1-3025) and identifying the cellular component(s) that binds to the compound to form a molecular complex, preferably through a specific interaction.

“Altered mitochondrial function” may refer to any condition or state, including those that accompany a disease associated with altered mitochondrial function, where any structure or activity that is directly or indirectly related to a mitochondrial function has been changed in a statistically significant manner relative to a control or standard. Altered mitochondrial function may have its origin in extramitochondrial structures or events as well as in mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

Additionally, altered mitochondrial function may include altered respiratory, metabolic or other biochemical or biophysical activity in one or more cells of a biological sample or a biological source. As non-limiting examples, markedly impaired ETC activity may be related to altered mitochondrial function, as may be generation of increased reactive oxygen species (ROS) or defective oxidative phosphorylation. As further examples, altered mitochondrial membrane potential, induction of apoptotic pathways and formation of a typical chemical and biochemical crosslinked species within a cell, whether by enzymatic or non-enzymatic mechanisms, may all be regarded as indicative of altered mitochondrial function. These and other non-limiting examples of altered mitochondrial function are contemplated by the present invention.

For instance, altered mitochondrial function may be related, interalia, to altered intracellular calcium regulation that may accompany loss of mitochondrial membrane electrochemical potential by intracellular calcium flux, by mechanisms that include free radical oxidation, defects in transmitochondrial membrane shuttles and transporters such as the adenine nucleotide transporter or the malate-aspartate shuttle, by defects in ATP biosynthesis, by impaired association of hexokinases and/or other enzymes with porin at the inner mitochondrial membrane, or by other events. Altered intracellular calcium regulation and/or collapse of mitochondrial inner membrane potential may result from direct or indirect effects of mitochondrial genes, gene products or related downstream mediator molecules and/or extramitochondrial genes, gene products or related downstream mediators, or from other known or unknown causes.

Thus, an “indicator of altered mitochondrial function” may be any detectable parameter that directly relates to a condition, process, pathway, dynamic structure, state or other activity involving mitochondria and that permits detection of altered mitochondrial function in a biological sample from a subject or biological source. According to non-limiting theory, altered mitochondrial function therefore may also include altered mitochondrial permeability to calcium or to mitochondrial molecular components involved in apoptosis (e.g., cytochrome c), or other alterations in mitochondrial respiration, or any other altered biological activity as provided herein that is a mitochondrially associated activity.

In certain preferred embodiments of the invention, an enzyme is the indicator of altered mitochondrial function as provided herein. The enzyme may be a mitochondrial enzyme, which may further be an ETC enzyme or a Krebs cycle enzyme. The enzyme may also be an ATP biosynthesis factor, which may include an ETC enzyme and/or a Krebs cycle enzyme, or other enzymes or cellular components related to ATP production as provided herein. A “non-enzyme” refers to an indicator of altered mitochondrial function that is not an enzyme (i.e., that is not a mitochondrial enzyme or an ATP biosynthesis factor as provided herein). In certain other preferred embodiments, an enzyme is a co-indicator of altered mitochondrial function. The following enzymes may not be indicators of altered mitochondrial function according to the present invention, but may be co-indicators of altered mitochondrial function as provided herein: citrate synthase (EC 4.1.3.7), hexokinase II (EC 2.7.1.1; see, e.g., Kruszynska et al. 1.998), cytochrome c oxidase (EC 1.9.3.1), phosphofructokinase (EC 2.7.1.11), glyceraldehyde phosphate dehydrogenase (EC 1.2.1.12), glycogen phosphorylase (EC 2.4.1.1) creatine kinase (EC 2.7.3.2), NADH dehydrogenase (EC 1.6.5.3), glycerol 3-phosphate dehydrogenase (EC 1.1.1.8), triose phosphate dehydrogenase (EC 1.2.1.12) and malate dehydrogenase (EC 1.1.1.37).

In other highly preferred embodiments, the indicator of altered mitochondrial function is any ATP biosynthesis factor as described below. In other preferred embodiments, the indicator is ATP production. In other preferred embodiments, the indicator of altered mitochondrial function may be mitochondrial mass or mitochondrial number. According to the present invention, mitochondrial DNA content may not be an indicator of altered mitochondrial function but may be a co-predictor of altered mitochondrial function or a co-indicator of altered mitochondrial function, as provided herein. In other preferred embodiments the indicator of altered mitochondrial function may be free radical production, a cellular response to elevated intracellular calcium or a cellular response to an apoptogen.

INDICATORS OF ALTERED MITOCHONDRIAL FUNCTION THAT ARE ENZYMES

As provided herein, in certain preferred embodiments, an altered biological activity comprises an indicator of altered mitochondrial function that may be an enzyme; such an enzyme may be a mitochondrial enzyme or an ATP biosynthesis factor that is an enzyme, for example an ETC enzyme or a Krebs cycle enzyme.

Reference herein to “enzyme quantity”, “enzyme catalytic activity” or “enzyme expression level” is meant to include a reference to any of a mitochondrial enzyme quantity, activity or expression level or an ATP biosynthesis factor quantity, activity or expression level; either of which may further include, for example, an ETC enzyme quantity, activity or expression level or a Krebs cycle enzyme quantity, activity or expression level. In the most preferred embodiments of the invention, an enzyme is a natural or recombinant protein or polypeptide that has enzyme catalytic activity as provided herein. Such an enzyme may be, by way of non-limiting examples, an enzyme, a holoenzyme, an enzyme complex, an enzyme subunit, an enzyme fragment, derivative or analog or the like, including a truncated, processed or cleaved enzyme.

A “mitochondrial enzyme” that may be an indicator of altered mitochondrial function as provided herein refers to a mitochondrial molecular component that has enzyme catalytic activity and/or functions as an enzyme cofactor capable of influencing enzyme catalytic activity. As used herein, mitochondria are comprised of “mitochondrial molecular components”, which may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or any covalently or non-covalently complexed combination of these components, or any other biological molecule that is a stable or transient constituent of a mitochondrion.

A mitochondrial enzyme that may be an indicator of altered mitochondrial function or a co-indicator of altered mitochondrial function as provided herein, or an ATP biosynthesis factor that may be an indicator of altered mitochondrial function as provided herein, may comprise an ETC enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths. Enzymol. 260:14; Ernster et al., 1981 J. Cell Biol. 91:227s-255s, and references cited therein).

A mitochondrial enzyme that may be an indicator of altered mitochondrial function as provided herein, or an ATP biosynthesis factor that may be an indicator of altered mitochondrial function as provided herein, may also comprise a Krebs cycle enzyme, which includes mitochondrial molecular components that mediate the series of biochemical/bioenergetic reactions also known as the citric acid cycle or the tricarboxylic acid cycle (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the α-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide, acetyl-coA carboxylase (ACC) and nucleoside diphosphokinase.

The methods of the present invention also pertain in part to the correlation of mitochondrial associated disease with an indicator of altered mitochondrial function that may be an ATP biosynthesis factor, an altered amount of ATP or an altered amount of ATP production.

An “ATP biosynthesis factor” refers to any naturally occurring cellular component that contributes to the efficiency of ATP production in mitochondria. Such a cellular component may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g., a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²P; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of altered mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (IU), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain preferred embodiments of the invention, enzyme catalytic activity may be expressed as units of substrate detectably converted by an enzyme to a product per unit time per unit total protein in a sample. In certain particularly preferred embodiments, enzyme catalytic activity may be expressed as units of substrate detectably converted by an enzyme to product per unit time per unit mitochondrial mass in a sample. In certain highly preferred embodiments, enzyme catalytic activity may be expressed as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, calorimetric, spectrophotometric, fluorimetric, immunometric or mass spectrometric procedures, or by other suitable means that will be readily apparent to a person having ordinary skill in the art.

In certain embodiments of the invention, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65,1987; Harlow and Lane, Antibodies: A Laboratory Manual, Cold Spring Harbor Laboratory, 1988; Haugland, 1996 Handbook of Fluorescent Probes and Research Chemicals-Sixth Ed., Molecular Probes, Eugene, Oreg.; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein). Spectroscopic methods may be used to detect dyes (including, for example, calorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

As noted above, enzyme catalytic activity of an ATP biosynthesis factor may further include other functional activities that lead to ATP production, beyond those involving covalent alteration of a substrate or reactant. For example by way of illustration and not limitation, an ATP biosynthesis factor that is an enzyme may refer to a transmembrane transporter molecule that, through its enzyme catalytic activity, facilitates the movement of metabolites between cellular compartments. Such metabolites may be-ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may, through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, according to the present invention, an ATP biosynthesis factor may include, as non-limiting examples, an ATP synthase, acetyl-coA carboxylase (ACC) a mitochondrial matrix protein and a mitochondrial membrane protein. Suitable mitochondrial membrane proteins include such mitochondrial components as the adenine nucleotide transporter (ANT; e.g., Fiore et al., 1998 Biochimie 80:137; Klingenberg 1985 Ann. N.Y. Acad. Sci. 456:279), the voltage dependent anion channel (VDAC, also referred to as porin; e.g., Manella, 1997 J. Bioenergetics Biomembr. 29:525), the malate-aspartate shuttle, the mitochondrial calcium uniporter (e.g., Litsky et al., 1997 Biochem. 36:7071), uncoupling proteins (UCP-1, -2, -3; see e.g., Jezek et al., 1998 Int. J. Biochem. Cell Biol. 30:1163), a hexokinase, a peripheral benzodiazepine receptor, a mitochondrial intermembrane creatine kinase, cyclophilin D, a Bcl-2 gene family encoded polypeptide, the tricarboxylate carrier (e.g., lacobazi et al., 1996 Biochim. Biophys. Acta 1284:9; Bisaccia et al., 1990 Biochim. Biophys. Acta 1019:250) and the dicarboxylate carrier (e.g., Fiermonte et al., 1998 J. Biol. Chem. 273:24754; Indiveri et al., 1993 Biochim. Biophys. Acta 1143:310; for a general review of mitochondrial membrane transporters, see, e.g., Zoratti et al., 1994 J. Bioenergetics Biomembr. 26:543 and references cited therein).

“Enzyme quantity” as used herein refers to an amount of an enzyme including mitochondrial enzymes or enzymes that are ATP biosynthesis factors as provided herein, or of another ATP biosynthesis factor, that is present, i.e., the physical presence of an enzyme or ATP biosynthesis factor selected as an indicator of altered mitochondrial function, irrespective of enzyme catalytic activity. Depending on the physicochemical properties of a particular enzyme or ATP biosynthesis factor, the preferred method for determining the enzyme quantity will vary. In the most highly preferred embodiments of the invention, determination of enzyme quantity will involve quantitative determination of the level of a protein or polypeptide using routine methods in protein chemistry with which those having skill in the art will be readily familiar, for example by way of illustration and not limitation, those described in greater detail below.

Accordingly, determination of enzyme quantity may be by any suitable method known in the art for quantifying a particular cellular component that is an enzyme or an ATP biosynthesis factor as provided herein, and that in preferred embodiments is a protein or polypeptide. Depending on the nature and physicochemical properties of the enzyme or ATP biosynthesis factor, determination of enzyme quantity may be by densitometric, mass spectrometric, spectrophotometric, fluorimetric, immunometric, chromatographic, electrochemical or any other means of quantitatively detecting a particular cellular component. Methods for determining enzyme quantity also include methods described above that are useful for detecting products of enzyme catalytic activity, including those measuring enzyme quantity directly and those measuring a detectable label or reporter moiety. In certain preferred embodiments of the invention, enzyme quantity is determined by immunometric measurement of an isolated enzyme or ATP biosynthesis factor. In certain preferred embodiments of the invention, these and other immunological and immunochemical techniques for quantitative determination of biomolecules such as an enzyme or ATP biosynthesis factor may be employed using a variety of assay formats known to those of ordinary skill in the art, including but not limited to enzyme linked immunosorbent assay (ELISA), radioimmunoassay (RIA), immunofluorimetry, immunoprecipitation, equilibrium dialysis, immunodiffusion and other techniques. (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston.) For example, the assay may be performed in a Western blot format, wherein a preparation comprising proteins from a biological sample is submitted to gel electrophoresis, transferred to a suitable membrane and allowed to react with an antibody specific for an enzyme or an ATP biosynthesis factor that is a protein or polypeptide. The presence of the antibody on the membrane may then be detected using a suitable detection reagent, as is well known in the art and described above.

INDICATORS OF ALTERED MITOCHONDRIAL FUNCTION THAT ARE CELLULAR RESPONSES TO ELEVATED INTRACELLULAR CALCIUM

According to certain embodiments of the present invention, a method is provided that comprises in pertinent part determining a biological activity of a mitochondrial polypeptide by monitoring intracellular calcium homeostasis and/or cellular responses to perturbations of this homeostasis, including physiological and pathophysiological calcium regulation. In particular, according to these embodiments, the method of the present invention is directed to comparing a cellular response to elevated intracellular calcium in a biological sample in the presence and absence of a candidate agent, or to comparing such a response in a sample from a subject known or suspected of having a disease associated with altered mitochondrial function with that of a control subject. The range of cellular responses to elevated intracellular calcium is broad, as is the range of methods and reagents for the detection of such responses. Many specific cellular responses are known to those having ordinary skill in the art; these responses will depend on the particular cell types present in a selected biological sample. It is within the contemplation of the present invention to provide a method comprising comparing a cellular response to elevated intracellular calcium, where such response is an indicator of altered mitochondrial function as provided herein. As non-limiting examples, cellular responses to elevated intracellular calcium include secretion of specific secretory products, exocytosis of particular preformed components, increased glycogen metabolism and cell proliferation (see, e.g., Clapham, 1995 Cell 80:259; Cooper, The Cell—A Molecular Approach, 1997 ASM Press, Washington, D.C.; Alberts, B., Bray, D., et al., Molecular Biology of the Cell, 1995 Garland Publishing, NY).

As a brief background, normal alterations of intramitochondrial Ca²⁺ are associated with normal metabolic regulation (Dykens, 1998 in Mitochondria & Free Radicals in Neurodegenerative Diseases, Beal, Howell and Bodis-Wollner, Eds., Wiley-Liss, New York, pp.29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physiol. 27: C755; Gunter et al., 1994, Am. J. Physiol. 267: 313). For example, fluctuating levels of mitochondrial free Ca²⁺ may be responsible for regulating oxidative metabolism in response to increased ATP utilization, via allosteric regulation of enzymes (reviewed by Crompton et al., 1993 Basic Res. Cardiol. 88: 513-523;) and the glycerophosphate shuttle (Gunter et al., 1994 J. Bioenerg. Biomembr. 26: 471).

Normal mitochondrial function includes regulation of cytosolic free calcium levels by sequestration of excess Ca²⁺ within the mitochondrial matrix. Depending on cell type, cytosolic Ca²⁺ concentration is typically 50-100 nM. In normally functioning cells, when Ca²⁺ levels reach 200-300 nM, mitochondria begin to accumulate Ca²⁺ as a function of the equilibrium between influx via a Ca²⁺ uniporter in the inner mitochondrial membrane and Ca²⁺ efflux via both Na⁺ dependent and Na⁺ independent calcium carriers. In certain instances, such perturbation of intracellular calcium homeostasis is a feature of diseases associated with altered mitochondrial function, regardless of whether the calcium regulatory dysfunction is causative of, or a consequence of, altered mitochondrial function.

Elevated mitochondrial calcium levels thus may accumulate in response to an initial elevation in cytosolic free calcium, as described above. Such elevated mitochondrial calcium concentrations in combination with reduced ATP or other conditions associated with mitochondrial pathology, can lead to collapse of mitochondrial inner membrane potential (see Gunter et al., 1998 Biochim. Biophys. Acta 1366:5; Rottenberg and Marbach, 1990, Biochim. Biophys. Acta 1016:87). Generally, in order to practice the subject invention methods, the extramitochondrial (cytosolic) level of Ca²⁺ in a biological sample is greater than that present within mitochondria. For example, in the case of type 2 diabetes mellitus (type 2 DM), mitochondrial or cytosolic calcium levels may vary from the above ranges and may range from, e.g., about 1 nM to about 500 mM, more typically from about 10 nM to about 100 μM and usually from about 20 nM to about 1 μM, where “about” indicates ±10%. A variety of calcium indicators are known in the art, including but not limited to, for example, fura-2 (McCormack et al., 1989 Biochim. Biophys. Acta 973:420); magfura-2; BTC (U.S. Pat. No. 5,501,980); fluo-3, fluo-4 and fluo-5N (U.S. Pat. No. 5,049,673); rhod-2; benzothiaza-1; and benzothiaza-2 (all of which are available from Molecular Probes, Eugene, Oreg.). These or any other means for monitoring intracellular calcium are contemplated according to the subject invention method for identifying a risk for type 2 DM.

For monitoring an indicator of altered mitochondrial function that is a cellular response to elevated intracellular calcium, compounds that induce increased cytoplasmic and mitochondrial concentrations of Ca²⁺, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals-Sixth Ed., Molecular Probes, Eugene, Oreg.). Accordingly, a person skilled in the art may readily select a suitable ionophore (or another compound that results in increased cytoplasmic and/or mitochondrial concentrations of Ca²⁺) and an appropriate means for detecting intracellular and/or intramitochondrial calcium for use in the present invention, according to the instant disclosure and to well known methods.

Ca²⁺ influx into mitochondria appears to be largely dependent, and may be completely dependent, upon the negative transmembrane electrochemical potential (Δψ) established at the inner mitochondrial membrane by electron transfer, and such influx fails to occur in the absence of Δψ even when an eight-fold Ca²⁺ concentration gradient is imposed (Kapus et al., 1991 FEBS Lett. 282:61). Accordingly, mitochondria may release Ca²⁺ when the membrane potential is dissipated, as occurs with uncouplers like 2,4-dinitrophenol and carbonyl cyanide p-trifluoro-methoxyphenylhydrazone (FCCP). Thus, according to certain embodiments of the present invention, collapse of Δψ may be potentiated by influxes of cytosolic free calcium into the mitochondria, as may occur under certain physiological conditions including those encountered by cells of a subject having type 2 DM. Detection of such collapse may be accomplished by a variety of means as provided herein.

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals-Sixth Ed., Molecular Probes, Eugene, Oreg., pp.266-274 and 589-594.). For example, by way of illustration and not limitation, the fluorescent probes 2-,4-dimethylaminostyryl-N-methyl pyridinium (DASPMI) and tetramethylrhodamine esters (such as, e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp.159-186 and references cited therein; and Molecular Probes On-line Handbook of Fluorescent Probes and Research Chemicals, at http://www.probes.com/handbook/toc.html). Other fluorescent detectable compounds that may be used in the invention include but are not limited to rhodamine 123, rhodamine B hexyl ester, DiOC₆(3), JC-1 [5,5′,6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Göttingen, Germany; see Sakanoue et al., 1997 J. Biochem. 121:29). Methods for monitoring mitochondrial membrane potential are also disclosed in U.S. application Ser. No. 09/161,172.

Mitochondrial membrane potential can also be measured by nonfluorescent means, for example by using TTP (tetraphenylphosphonium ion) and a TTP-sensitive electrode (Kamo et al., 1979 J. Membrane Biol. 49:105; Porter and Brand, 1995 Am. J. Physiol. 269:R1213). Those skilled in the art will be able to select appropriate detectable compounds or other appropriate means for measuring Δψm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.)

Exquisite sensitivity to extraordinary mitochondrial accumulations of Ca²⁺ that result from elevation of intracellular calcium, as described above, may also characterize type 2 DM. Such mitochondrial sensitivity may provide an indicator of altered mitochondrial function according to the present invention. Additionally, a variety of physiologically pertinent agents, including hydroperoxide and free radicals, may synergize with Ca²⁺ to induce collapse of Δψ (Novgorodov et al., 1991 Biochem. Biophys. Acta 1058: 242; Takeyama et al., 1993 Biochem. J. 294:719; Guidox et al., 1993 Arch. Biochem. Biophys. 306:139).

INDICATORS OF ALTERED MITOCHONDRIAL FUNCTION THAT ARE CELLULAR RESPONSES TO APOPTOGENIC STIMULI

Turning to another aspect, the present invention relates to the correlation of diseases associated with altered mitochondrial function with an indicator of altered mitochondrial function, involving programmed cell death or apoptosis. In particular, according to this aspect, the present invention is directed to a method comprising comparing a cellular response to an apoptosis-inducing (“apoptogenic”) stimulus in a biological sample from (i) a subject believed to be at risk for disease, and (ii) a control subject. The range of cellular responses to various known apoptogenic stimuli is broad, as is the range of methods and reagents for the detection of such responses. It is within the contemplation of the present invention to provide a method for identifying a risk for disease by comparing a cellular response to an apoptogenic stimulus, where such response is an indicator of altered mitochondrial function as provided herein.

By way of background, mitochondrial dysfunction is thought to be critical in the cascade of events leading to apoptosis in various cell types (Kroemer et al., FASEB J. 9:1277-87, 1995). Altered mitochondrial physiology may be among the earliest events in programmed cell death (Zamzami et al., J. Exp. Med. 182:367-77, 1995; Zamzami et al., J. Exp. Med. 181:1661-72, 1995) and elevated reactive oxygen species (ROS) levels that result from such altered mitochondrial function may initiate the apoptotic cascade (Ausserer et al., Mol. Cell. Biol. 14:5032-42,1994). In several cell types, reduction in the mitochondrial membrane potential (Δψm) precedes the nuclear DNA degradation that accompanies apoptosis. In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS, may occur in certain diseases associated with altered mitochondrial function (e.g., type 2 DM) and may further induce pathogenetic events via apoptotic mechanisms.

Oxidatively stressed mitochondria may release a pre-formed soluble factor that can induce chromosomal condensation, an event preceding apoptosis (Marchetti et al., Cancer Res. 56:2033-38, 1996). In addition, members of the Bcl-2 family of anti-apoptosis gene products are located within the outer mitochondrial membrane (Monaghan et al., J. Histochem. Cytochem. 40:1819-25,1992) and these proteins appear to protect membranes from oxidative stress (Korsmeyer et al, Biochim. Biophys. Act. 1271:63, 1995). Localization of Bcl-2 to this membrane appears to be indispensable for modulation of apoptosis (Nguyen et al., J. Biol. Chem. 269:16521-24, 1994). Thus, changes in mitochondrial physiology may be important mediators of apoptosis.

Altered mitochondrial function, may therefore lower the threshold for induction of apoptosis by an apoptogen. A variety of apoptogens are known to those familiar with the art (see, e.g., Green et al., 1998 Science 281:1309 and references cited therein) and may include by way of illustration and not limitation: tumor necrosis factor-alpha (TNF-α); Fas ligand; glutamate; N-methyl-D-aspartate (NMDA); interleukin-3 (IL-3); herbimycin A (Mancini et al., 1997 J. Cell. Biol. 138:449-469); paraquat (Costantini et al., 1995 Toxicology 99:1-2); ethylene glycols; protein kinase inhibitors, such as, e.g. staurosporine, calphostin C, caffeic acid phenethyl ester, chelerythrine chloride, genistein; 1-(5-isoquinolinesulfonyl)-2-methylpiperazine; N-[2-((p-bromocinnamyl)amino)ethyl]-5-5-isoquinolinesulfonamide; KN-93; quercitin; d-erythro-sphingosine derivatives; UV irradiation; ionophores such as, e.g.: ionomycin and valinomycin; MAP kinase inducers such as, e.g.: anisomycin, anandamine; cell cycle blockers such as, e.g.: aphidicolin, colcemid, 5-fluorouracil, homoharringtonine; acetylcholinesterase inhibitors such as, e.g. berberine; anti-estrogens such as, e.g.: tamoxifen; pro-oxidants, such as, e.g.,: tert-butyl peroxide, hydrogen peroxide; free radicals such as, e.g., nitric oxide; inorganic metal ions, such as, e.g., cadmium; DNA synthesis inhibitors such as, e.g.: actinomycin D; DNA intercalators such as, e.g., doxorubicin, bleomycin sulfate, hydroxyurea, methotrexate, mitomycin C, camptothecin, daunorubicin; protein synthesis inhibitors such as, e.g., cycloheximide, puromycin, rapamycin; agents that affect microtubulin formation or stability such as, e.g.: vinblastine, vincristine, colchicine, 4-hydroxyphenylretinamide, paclitaxel; Bad protein, Bid protein and Bax protein (see, e.g., Jurgenmeier et al., 1998 Proc. Nat. Acad. Sci. USA 95:4997-5002 and references cited therein); calcium and inorganic phosphate (Kroemer et al., 1998 Ann. Rev. Physiol. 60:619).

In one embodiment of the subject invention method wherein the indicator of altered mitochondrial function is a cellular response to an apoptogen, cells in a biological sample that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another embodiment of the subject invention method wherein the indicator of altered mitochondrial function is a cellular response to an apoptogen, cells in a biological sample may be assayed for translocation of cell membrane phosphatidylserine (PS) from the inner to the outer leaflet of the plasma membrane, which may be detected, for example, by measuring outer leaflet binding by the PS-specific protein annexin. (Martin et al., J. Exp. Med. 182:1545,1995; Fadok et al., J. Immunol. 148:2207,1992.) In still another embodiment of this aspect of the invention, a cellular response to an apoptogen is determined by an assay for induction of specific protease activity in any member of a family of apoptosis-activated proteases known as the caspases (see, e.g., Green et al., 1998 Science 281:1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., 1997 J. Neurosci. 17:6165). The synthetic peptide Z-Tyr-Val-Ala-Asp-AFC (SEQ ID NO: ______;), wherein “Z” indicates a benzoyl carbonyl moiety and AFC indicates 7-amino-4-trifluoromethylcoumarin (Kluck et al., 1997 Science 275:1132; Nicholson et al., 1995 Nature 376:37), is one such substrate. Other non-limiting examples of substrates include nuclear proteins such as U1-70 kDa and DNA-PKcs (Rosen and Casciola-Rosen, 1997 J. Cell. Biochem. 64:50; Cohen, 1997 Biochem. J. 326:1).

As described above, the mitochondrial inner membrane may exhibit highly selective and regulated permeability for many small solutes, but is impermeable to large (>˜10 kDa) molecules. (See, e.g., Quinn, 1976 The Molecular Biology of Cell Membranes, University Park Press, Baltimore, Md.). In cells undergoing apoptosis, however, collapse of mitochondrial membrane potential may be accompanied by increased permeability permitting macromolecule diffusion across the mitochondrial membrane. Thus, in another embodiment of the subject invention method wherein the indicator of altered mitochondrial function is a cellular response to an apoptogen, detection of a mitochondrial protein, for example cytochrome c that has escaped from mitochondria in apoptotic cells, may provide evidence of a response to an apoptogen that can be readily determined. (Liu et al., Cell 86:147, 1996) Such detection of cytochrome c may be performed spectrophotometrically, immunochemically or by other well established methods for determining the presence of a specific protein.

For instance, release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/lonization (SELDI™) system (Ciphergen, Palo Alto, Calif.) may be utilized to detect cytochrome c release from mitochondria in apoptogen treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI™ mass spectrometer.

A person having ordinary skill in the art will readily appreciate that there may be other suitable techniques for quantifying apoptosis, and such techniques for purposes of determining an indicator of altered mitochondrial function that is a cellular response to an apoptogenic stimulus are within the scope of the methods provided by the present invention.

As noted above, an increasing number of diseases, disorders and conditions have been identified as diseases associated with altered mitochondrial function as provided herein, such that given the present disclosure and the state of the art with respect to methods for assessing mitochondrial function and with respect to clinical signs and symptoms of such diseases, the person having ordinary skill in the art can readily determine criteria for establishing a statistically significant deviation from a normal range for one or more parameters that are appropriate to the definition of the disease, in order to establish that a disease associated with altered mitochondrial function is present. As an illustrative example, where it is desirable to determine whether or not a subject or biological source falls within clinical parameters indicative of type 2 diabetes mellitus, signs and symptoms of type 2 diabetes that are accepted by those skilled in the art may be used to so designate a subject or biological source, for example clinical signs referred to in Gavin et al. (Diabetes Care 22(suppl. 1):S5-S19,1999, American Diabetes Association Expert Committee on the Diagnosis and Classification of Diabetes Mellitus) and references cited therein, or other means known in the art for diagnosing type 2 diabetes. Similarly, those familiar with the art will be aware of art accepted criteria for determining the presence of other diseases associated with altered mitochondrial function as provided herein.

Hence, the person having ordinary skill in the art can “correlate” one or more parameters described herein (e.g., mitochondrial functions) with such a disease associated with altered mitochondrial function, in view of the present disclosure and based on familiarity with the art. Briefly, statistically significant deviation from a normal, disease-free range for any of a number of clinical signs and symptoms and/or criteria for mitochondrial function, permits determination of the statistically significant coincidence of such parameter(s) with disease. Such deviation can further be confirmed, for instance, by comparing the same parameters and criteria that are detected in disease to those in a suitable control sample, in this case a control derived from a subject known to be free of a risk for having, or presence of, such disease.

Accordingly, given the disclosure of the instant application, and in particular the identification of the polypeptide sequences set forth in SEQ ID NOS:1-3025 as belonging to a defined human mitochondrial proteome, the present invention provides a control set of polypeptides such that a sample may be analyzed for the presence of at least one modified polypeptide as described herein, in order to so “correlate” such modification with a disease associated with altered mitochondrial function. Establishing such a correlation then provides a target for screening assays to identify an agent suitable for therapeutic intervention, i.e., an agent that beneficially counteracts the disease-associated alteration in mitochondrial function. Without wishing to be bound by theory, a target for therapeutic intervention preferably contributes to the pathogenesis of disease by exhibiting undesirably altered biological activity, such that a therapeutic agent reverses such alteration to a control range. The invention need not, however, be so limited, as even in situations where the target identified according to the subject invention method is a surrogate marker of disease, such a target nevertheless may be restored to a normal control range by a therapeutic agent regardless of whether the interaction is direct, in a manner that ameliorates disease. In certain embodiments the invention further provides for determination of altered biological activity in such a modified polypeptide, as also described herein.

According to the present invention, there are provided compositions and methods for the identification of differential protein expression at the organellar proteome level (e.g., the mitochondrial proteome), in a sub-proteomic, complex mixture of proteins or at the level of a single targeted protein. The invention thus relates in pertinent part to the unexpected advantages associated with the unique physicochemical properties of particular organelle-derived (e.g., mitochondria) polypeptides, peptides (e.g., peptide fragments) and proteins, in conjunction with biochemical (including immunochemical) methods, modern spectrometry and protein bioinformatics software tools to identify peptides and proteins that are detected as differentially expressed products, and to identify previously unrecognized peptides and proteins as molecular components of a particular organelle (e.g., mitochondrial molecular components as provided herein).

The invention also relates in pertinent part to the surprising advantages offered by the use of an organelle enriched sample fraction (e.g., a mitochondria enriched sample as provided herein). Determining the pattern of differential protein expression (e.g., absence or presence of one or more particular proteins in a sample; structural modification of a particular protein; or other altered expression such as a statistically significant increase or decrease in the amount of one or more particular proteins in a sample when normalized to a control) at the peptide and/or protein level in a complex protein mixture obtained from a biological sample as provided herein (i.e., at the proteomic level) provides, in certain embodiments, targets for drug screening assays and for therapeutic intervention in specific disease states. Accordingly, in certain embodiments the invention provides methods for evaluating the effects of candidate therapeutic agents (e.g., drugs or biological stimuli as provided herein) on biological activity of a mitochondrial protein, for example, where the protein exhibits altered biological activity due to one or more of a modification such as a mutation (insertion, deletion and/or substitution of one or more amino acids), a posttranslational modification or an altered level of protein expression. Thus, in certain embodiments, such candidate agents may cause one or more specific alterations (e.g., increases or decreases in a statistically significant manner) in the biological activity of a mitochondrial protein, preferably in some beneficial fashion.

As also noted elsewhere herein, certain embodiments of the invention relate in pertinent part to isolating at least one mitochondrial polypeptide according to any of a variety of biochemical separation methodologies for isolating a polypeptide as known in the art and as provided herein (see, e.g., Scopes, 1987 Protein Purification: Principles and Practice, Springer-Verlag, NY; Deutscher, 1990 Meths. Enzymol. Vol. 182; Nilsson et al., 2000 Mass Spectrom. Rev. 19:390; Godovac-Zimmermann et al., 2001 Mass Spectrom. Rev. 20:1; Gatlin et al., 2000 Anal. Chem. 72:757; Link et al., 1999 Nat. Biotechnol. 17:676). Hence, as provided herein and as known to the art, such methodologies for isolating a mitochondrial polypeptide may exploit physicochemical and hydrodynamic properties of the polypeptide, including, for example, the approximate apparent molecular mass of the polypeptide, the amino acid sequence of the polypeptide, and in certain contemplated embodiments, the apparent approximate isolelectric focusing point of the polypeptide.

As is well known to those having ordinary skill in the art, variability in biological sample source and condition, extraction reagents and methods, separation media and instrumentation, analytical apparatus and the like, may account for differences in values observed for such properties of polypeptides as molecular mass and isoelectric focusing point. Hence, it will be understood that an “apparent” molecular mass or isoelectric focusing point refers to that which is detected in a particular rendition of a particular isolation procedure, although the value detected for such a parameter may vary among separate isolations; similarly those familiar with the art will appreciate that from among the variables listed above, including imprecision in instrumentation, apparent values may vary in a manner that renders a particular value that is detected only an “approximation” of the actual parameter being measured. Thus, according to certain embodiments of the present invention a mitochondrial polypeptide may be isolated on the basis of approximate apparent molecular mass, apparent approximate isoelectric focusing point and/or amino acid sequence, which parameters may be susceptible to some variability for reasons discussed above but which, in any event, will permit isolation of such a polypeptide as provided herein.

The isolated polypeptide is then contacted with a proteolytic agent to generate a plurality of derivative peptide fragments, from which a mass spectrum can be generated to permit determination of the presence, amount or structure (e.g., level) of the polypeptide in the sample, which may then be compared to similarly obtained levels of a mitochondrial polypeptide obtained from other samples.

In an effort to better understand the molecular details of mitochondrial dysfunction as a contributing factor in disease, a high-resolution map of the human mitochondrial proteome is disclosed herein using human heart tissue as the source of isolated mitochondria, which are further enriched on metrizamide density gradients, solubilized and fractionated using sucrose density gradients. Although a protein map was previously generated using an only partially enriched mitochondrial fraction from human placenta (Rabilloud et al., 1998 Electrophor. 19:1006), no reliable database cataloguing mitochondrial proteins is currently available (cf., e.g., Koc et al., 2000 J. Biol. Chem. 275:32585; Lopez et al., 2000 Electrophor. 21:3427). Typically, mitochondria may be obtained from brain, heart, skeletal muscle or liver, where they are most abundant, although other sources (e.g., blood platelets) may also be used. According to the present invention there is provided a framework for investigating mitochondrial proteins, including identifying previously unrecognized mitochondrial proteins (e.g., novel proteins or known proteins not previously known to exist as mitochondrial molecular components) as well as those that are modified as provided herein as a correlate of disease, by mapping the human heart mitochondrial proteome. As described in greater detail in the Examples, mitochondrial proteins in distinct sucrose density gradient fractions were separated by one-dimensional polyacrylamide gel electrophoresis, and isolated proteins recovered from gels were analyzed as described below using matrix assisted laser desorption ionization (MALDI) and MALDI-post source decay (MALDI-PSD) techniques. (For other MS methods for proteins, see, e.g., Godovac-Zimmermann et al., 2001 Mass Spectromet. Rev. 20:1-57; Nilsson et al., 2000 Mass Spectromet. Rev. 19:390-397.) Over 1400 proteins were identified in the NCBI (http://www.ncbi.nim.nih.gov/Entrez/) and GenPept (http://www.ncbi.nlm.nih.gov/Entrez/protein.html) databases. Alternative databases for identifying protein sequences are known to the art and include, for example, Swissprot (http://www.expasy.ch/sprot/sprot-top.html), and owl (http://www.biochem.ucl.ac.uk/bsm/dbbrowser/OWL/OWL.html.) The data set so obtained provides for the identification of proteins present in mitochondria from human heart, a bioenergetically active tissue.

As described in greater detail below, the present invention is also directed in pertinent part to the use of mass spectrometry (MS), and in particular to the use of matrix assisted laser desorption ionization time-of-flight (MALDI-TOF) mass spectrometry, for the analysis of mitochondrial proteins and peptides obtained from a subject or biological source as provided herein.

In particularly preferred embodiments of the present invention, all or a portion of a protein fraction derived from a biological sample as provided herein may be contacted with one or more proteolytic agents under conditions and for a time sufficient to generate a plurality of peptide fragments derived from the protein fraction. Peptide fragments are typically continuous portions of a polypeptide chain derived from a protein of the protein fraction, which portions may be up to about 100 amino acids in length, preferably up to about 50 amino acids in length, more preferably up to about 30 amino acids in length, and still more preferably up to about 15-20 amino acids in length. In particularly preferred embodiments peptide fragments are 10-15 amino acids in length, and in other preferred embodiments peptide fragments may be 2-12 amino acids long.

A variety of proteolytic agents and suitable conditions for using them are known in the art, any of which may be useful according to certain embodiments of the present invention wherein peptide fragments are generated. Particularly preferred are proteolytic agents that are proteolytic enzymes or proteases, for example trypsin, Glu-C protease (Staphylococcal V8 protease), Lys-C protease, Arg-C protease, or other proteases known in the art to cleave peptides at specific amino acid linkages, typically at a relatively limited number of cleavage sites within a protein or polypeptide. Other useful proteolytic agents that are proteolytic enzymes include serine proteases, for example, chymotrypsin, elastase and trypsin; thiolproteases, such as papain or yeast proteinase B; acid proteases, including, e.g., pepsin or cathepsin D; metalloproteinases (e.g., collagenases, microbial neutral proteinases); carboxypeptidases; N-terminal peptidases or any other proteolytic enzymes that those having ordinary skill in the art will recognize may be employed to generate peptide fragments as provided herein (see, e.g., Bell, J. E. and Bell, E. T., Proteins and Enzymes, 1988 Prentice-Hall, Englewood Cliffs, N.J.; Worthington Enzyme Manual, V. Worthington, ed., 1993 Worthington Biochemical Corp., Freehold, N.J.).

Alternatively, in certain embodiments it may be desirable to use proteolytic agents that are chemical agents, for example HCl, CNBr, formic acid, N-bromosuccinimide, BNPS-skatole, o-iodosobenzoic acid/p-cresol, Cyssor, 2-nitro-5-thiocyanobenzoic acid, hydroxylamine, pyridine/acetic acid or other chemical cleavage procedures (see, e.g., Bell and Bell, 1988, and references cited therein).

As noted above, oxidative damage to proteins, such as protein modification that results from reactive free radical activity in biological systems, is an underlying feature in the pathogenesis of a number of diseases. Accordingly, a disease associated with altered mitochondrial function, for example a disease associated with altered mitochondrial constitution or composition (e.g., a disorder or condition characterized by statistically significant alterations in the quantity, structure and/or activity of one or more mitochondrial molecular components as provided herein) may also include a “disease associated with oxidative modification of a protein”, such as any disease in which at least one protein or peptide is oxidatively (e.g., covalently) and, in most cases, inappropriately modified. In highly preferred embodiments, at least one protein or peptide in a subject or biological source having a disease associated with oxidative modification of a protein includes a mitochondrial protein that has undergone disease-associated oxidative damage. Thus, such a disease may have a basis in a respiratory or metabolic or other defect, whether mitochondrial or extramitochondrial in origin. Diseases associated with oxidative modification of proteins may include Alzheimer's disease (AD), diabetes mellitus, Parkinson's disease, amyotrophic lateral sclerosis (ALS), atherosclerosis and other degenerative and inflammatory diseases. Those familiar with the art will be aware of clinical criteria for diagnosing certain of these diseases, which diagnostic criteria are augmented in view of the subject invention methods and compositions.

As described in greater detail in the Examples, certain embodiments of the invention contemplate the unexpected discovery that a mitochondrial protein or peptide containing tryptophan may be oxidatively modified to yield proteins or peptides containing this modified amino acid, although the invention is not intended to be so limited and as described herein contemplates mitochondrial proteins and peptides comprising a wide variety of other amino acids that may be oxidatively modified, according to oxidation reactions such as those described, for example, in Halliwell and Gutteridge (Free Radicals in Biology and Medicine, 1989 Clarendon Press, Oxford, UK). As described below, a number of mitochondrial proteins have been identified in which at least one tryptophan residue was doubly oxidized, thereby undergoing conversion to N-formylkynurenine. Accordingly, in certain embodiments the invention contemplates determination of a modified polypeptide (e.g., SEQ ID NOS:1-3025) comprising an oxidative modification that may, in certain further embodiments comprise an oxidized trytophan residue, which may in certain still further comprise N-formylkynurenine. Identification and determination of oxidative modification of tryptophan in proteins and peptides are well known to those familiar with the art (e.g., Halliwell and Gutteridge, pages 93-97; 315-320; 413-429).

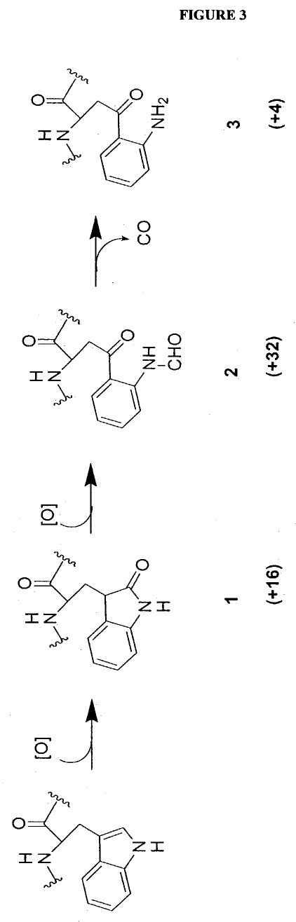

For instance, the oxidation of tryptophan to N-formylkynurenine in proteins has been known for over 35 years since Previero et al. described it in hen's egg-white lysozyme in anhydrous formic acid (1967 J. Mol. Biol. 24:261). Kuroda et al. (1975 J. Biochem. (Tokyo) 78:641) subsequently found inactivation of lysozyme by ozone in aqueous solution occurred only when one critical tryptophan residue was oxidized, thus providing the first evidence that oxidation of a specific tryptophan residue can impair enzyme function. These early reports relied on identification of the tryptophan oxidation products by characteristic electronic absorption spectra. Finley et al. (1998 Protein Sci. 7:2391) exposed α-crystallin from bovine lens tissue to Fenton chemistry in vitro and separated the component tryptic peptides by HPLC. Tandem MS/MS spectrometry was used to identify oxidized amino acid sites by +16, +32 and +4 u increases in the molecular mass of peptide fragment ions containing tryptophan residues. Structures corresponding to those mass shifts are shown in FIG. 3. More recently Thiede et al. (2000 Rapid Commun. Mass Spectrom. 14:496) described oxidatively modified tryptophan residues in peptides from human Jurkat T lymphoblastoid cells. These workers described oxidatively modified tryptophan in a peptide which, as shown by the Examples provided herein, shares structure with a similar peptide derived from the mitochondrial voltage dependent anion channel-1 (VDAC1, e.g., SEQ ID NO:2559) polypeptide (see Table 3, KLETAVNLAWTAGNSNTR). Certain embodiments of the present invention therefore contemplate expressly excluding determination of the peptide KLETAVNLAWTAGNSNTR which comprises oxidatively modified tryptophan, certain other embodiments contemplate expressly excluding an oxidatively modified VDAC1 polypeptide, and certain other embodiments of the present invention therefore contemplate expressly excluding a disease associated with altered mitochondrial function that is T-cell lymphoma or leukemia.

In order to determine whether a mitochondrial component may contribute to a particular disease associated with oxidative modification of a protein, it may be useful to construct a model system for diagnostic tests and for screening candidate therapeutic agents in which the nuclear genetic background may be held constant while the mitochondrial genome is modified. It is known in the art to deplete mitochondrial DNA from cultured cells to produce η⁰ cells, thereby preventing expression and replication of mitochondrial genes and inactivating mitochondrial function. It is further known in the art to repopulate such η⁰ cells with mitochondria derived from foreign cells in order to assess the contribution of the donor mitochondrial genotype to the respiratory phenotype of the recipient cells. Such cytoplasmic hybrid cells, containing genomic and mitochondrial DNAs of differing biological origins, are known as cybrids. See, for example, International Publication Number WO 95/26973 and U.S. Pat. No. 5,888,498 which are hereby incorporated by reference in their entireties, and references cited therein.

According to the present invention, a level of at least one mitochondrial protein or peptide is determined in a biological sample from a subject or biological source. For subjects that are asymptomatic, that exhibit a pre-disease phenotype or that meet clinical criteria for having or being at risk for having a particular disease, such determination may have prognostic and/or diagnostic usefulness. For example, where other clinical indicators of a given disease are known, levels of at least one mitochondrial protein or peptide in subjects known to be free of a risk or presence of such disease based on the absence of these indicators may be determined to establish a control range for such level(s). The levels may also be determined in biological samples obtained from subjects suspected of having or being at risk for having the disease, and compared to the control range determined in disease free subjects. Those having familiarity with the art will appreciate that there may be any number of variations on the particular subjects, biological sources and bases for comparing levels of at least one mitochondrial protein or peptide that are useful. beyond those that are expressly presented herein, and these additional uses are within the scope and spirit of the invention.

For instance, determination of levels of at least one mitochondrial protein or peptide may take the form of a prognostic or a diagnostic assay performed on a skeletal muscle biopsy, on whole blood collected from a subject by routine venous blood draw, on buffy coat cells prepared from blood or on biological samples that are other cells, organs or tissue from a subject. Alternatively, in certain situations it may be desirable to construct cybrid cell lines using mitochondria from either control subjects or subjects suspected of being at risk for a particular disease associated with oxidative modification of proteins. Such cybrids may be used to determine levels of at least one mitochondrial peptide or protein for diagnostic or predictive purposes, or as biological sources for screening assays to identify agents that may be suitable for treating the disease based on their ability to alter (e.g., to increase or decrease in a statistically significant manner) the levels of at least one mitochondrial protein or peptide in treated cells.

In one embodiment of this aspect of the invention, therapeutic agents or combinations of agents that are tailored to effectively treat an individual patient's particular disease may be identified by routine screening of candidate agents on cybrid cells constructed with the patient's mitochondria. In another embodiment, a method for identifying subtypes of the particular disease is provided, for example, based on differential effects of individual candidate agents on cybrid cells constructed using mitochondria from different subjects diagnosed with the same disease.

MALDI

As noted above, in certain preferred embodiments of the present invention there is provided a method for identifying at least one mitochondrial protein comprising generating a mass spectrum of a mitochondrial polypeptide-derived peptide fragment, wherein the mass spectrum is preferably generated using MALDI-TOF. By way of background, in 1987, matrix-assisted laser desorption/ionization mass spectrometry (MALDI) was introduced by Hillenkamp and Karas, and since has become a very powerful bioanalytical tool (Anal. Chem. 60:2288-2301,1988; see also Burlingame et al., Anal. Chem. 68:599-651, 1996 and references cited therein). The success of MALDI in the area of protein science can be attributed to several factors. The greatest of these is that MALDI can be rapidly (˜5 minutes) applied as an analytical technique to analyze small quantities of virtually any protein (practical sensitivities of ˜1 pmole protein loaded into the mass spectrometer). The technique is also extremely accurate. Beavis and Chait demonstrated that the molecular weights of peptides and proteins can be determined to within ˜0.01% by using methods in which internal mass calibrants (x-axis calibration) are introduced into the analysis (Anal. Chem. 62:1836-40, 1990). MALDI can also be made quantitative using a similar method in which internal reference standards are introduced into the analysis for ion signal normalization (y-axis calibration). Quantitative determination of proteins and peptides is possible using this approach with accuracies on the order of ˜10% (Nelson et al., Anal. Chem. 66:1408-15, 1994). Finally, MALDI is extremely tolerant of large molar excesses of buffer salts and, more importantly, the presence of other proteins.

With the high tolerance towards buffer salts and other biomolecular components comes the ability to directly analyze complex biological mixtures. Many examples exist where MALDI is used to directly analyze the results of proteolytic or chemical digestion of polypeptides (see Burlingame et al., supra). Other examples extend to elucidating post-translational modifications (namely carbohydrate type and content), a process requiring the simultaneous analysis of components present in a heterogeneous glycoprotein mixture (Sutton et al., Techniques in Protein Chemistry III, Angeletti, Ed., Academic Press, Inc., New York, pp.109-116,1993). Arguably, the most impressive use of direct mixture analysis is the screening of natural biological fluids. In that application, proteins are identified, as prepared directly from the host fluid, by detection at precise and characteristic mass-to-charge (m/z) values (Tempst et al., Mass Spectrometry in the Biological Sciences, Burlingame and Carr, Ed., Humana Press, Totowa, N.J., p.105, 1996).

The use of an affinity ligand-derivatized support to selectively retrieve a target analyte specifically for MALDI analysis was first demonstrated by Hutchens and Yip (Rapid Commun. Mass Spectrom. 7:576-80, 1993). Those investigators used single-stranded DNA-derivatized agarose beads to selectively retrieve a protein, lactoferrin, from pre-term infant urine by incubating the beads with urine. The agarose beads were then treated as the MALDI analyte—a process involving mixing with a solution-phase MALDI matrix followed by deposition of the mixture on a mass spectrometer probe. MALDI then proceeded in the usual manner. Results indicated that the derivatized beads selectively retrieved and concentrated the lactoferrin; enough so to enable ion signal in the MALDI mass spectrum adequate to unambiguously identify the analyte at the appropriate m/z value (81,000 Da). A number of variations on this approach have since been reported. These include the use of immunoaffinity precipitation for the MALDI analysis of transferrins in serum (Nakanishi et al., Biol. Mass Spectrom. 23:230-33,1994), screening of ascites for the production of monoclonal antibodies (Papac et al., Anal. Chem. 66:2609-13, 1994), and the identification of linear epitope regions within an antigen (Zhao et al., Anal. Chem. 66:3723-26,1994). Even more recently, the affinity capture approaches have been made rigorously quantitative by incorporating mass-shifted variants of the analyte into the analysis (Nelson et al. Anal. Chem. 67:1153-58,1995). The variants are retained throughout the analysis (in the same manner as the true analyte) and observed as unique (resolved) signals in the MALDI mass spectrum. Quantification of the analyte is performed by equating the relative ion signals of the analyte and variant to an analyte concentration.

Suitable mass spectrometers include, but are not limited to, a magnetic sector mass spectrometer, a Fourier transform ion cyclotron resonance (FTICR) mass spectrometer, a quadrupole (rods or ion trap) mass spectrometer and a time-of-flight (TOF) mass spectrometer, and/or various hybrid instruments comprising combinations of any two or more of such types of mass analyzer (e.g., quadrupole/orthogonal TOF, Qq/TOF, TOF/TOF, etc.). In a preferred embodiment, the mass spectrometer is a time TOF mass spectrometer.

Since large molecules, such as peptides and proteins, are generally too large to be desorbed/ionized intact, a matrix is used to assist laser desorption/ionization of the same. This technique is referred to as matrix assisted laser desorption/ionization or (MALDI), and the matrix agent is referred to as a “MALDI matrix.” In short, the analyte is contacted with a suitable MALDI matrix and allowed to crystallize. Suitable MALDI matrix materials are known to those skilled in this field, and include, for example, derivatives of cinnamic acid such as α-cyano-4-hydroxycinnamic acid (ACCA) and sinapinic acid (SA).

A first criterion to performing mass spectrometry on the analyte captured by the interactive surface is the generation of vapor-phase ions. In the practice of this invention, such species are generated by desorption/ionization techniques. Suitable techniques include desorption/ionization methods derived from impact of particles with the sample. These methods include fast atom bombardment (FAB—impact of neutrals with a sample suspended in a volatile matrix), secondary ion mass spectrometry (SIMS—impact of keV primary ions generating secondary ions from a surface), liquid SIMS (LSIMS—like FAB except the primary species is an ion), plasma desorption mass spectrometry (like SIMS except using MeV primary ions), massive cluster impact (MCI—like SIMS using large cluster primary ions), laser desorption/ionization (LDI—laser light is used to desorb/ionize species from a surface), and matrix-assisted laser desorption/ionization (MALDI—like LDI except the species are desorbed/ionized from a matrix capable of assisting in the desorption and ionization events). Any of the aforementioned desorption/ionization techniques may be employed in the practice of the present invention. In a preferred embodiment, LDI is employed, and in a more preferred embodiment, MALDI is utilized. For matrix assisted laser desorption ionization/time of flight (MALDI-TOF) analysis or other MS (mass spectrometry) techniques known to those skilled in the art, see, for example, U.S. Pat. Nos. 5,622,824, 5,605,798 and 5,547,835. Alternatively, other soft-ionization mechanisms that are not based on particle bombardment but that are also capable of ionizing peptides and/or proteins could be employed. Such methods include electrospray ionization (ESI, liquid flow containing analyte sprayed from a nozzle or needle at high voltage) or atmospheric pressure ionzation (API).

Screening Assays and Agents

In certain embodiments, the present invention provides a method of identifying an agent for treating a disease associated with altered mitochondrial function, comprising (a) contacting a candidate agent with a biological sample from a subject having a disease associated with altered mitochondrial function, wherein the sample comprises at least one polypeptide that exhibits altered biological activity which accompanies the disease and wherein the polypeptide is (i) a polypeptide having an amino acid sequence as set forth in any one of SEQ ID NOS 1-3025, or (ii) a modified polypeptide that comprises at least one modification to a polypeptide having an amino acid sequence as set forth in any one of SEQ ID NOS 1-3025; and (b) determining an increase or decrease in the altered biological activity of the polypeptide in the presence of the candidate agent relative to the level-of the altered biological activity in the absence of the candidate agent, and therefrom identifying an agent for treating a disease associated with altered mitochondrial function.

Candidate agents for use in these and related methods of screening for a modulator of mitochondrial protein or peptide according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, and then assayed for their ability to increase or decrease the level of at least one indicator of altered mitochondrial function.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures, and tested for their influence on an indicator of altered mitochondrial function, according to the present disclosure.

The present invention provides compositions and methods that are useful in pharmacogenomics, for the classification and/or stratification of a subject or patient population. In one embodiment, for example, such stratification may be achieved by identification in a subject or patient population of one or more distinct profiles of at least one mitochondrial protein or peptide that is modified (e.g., an altered expression level, altered amino acid sequence, altered posttranslational modification or an oxidative modification) or in which the biological activity is altered and that correlates with a particular disease associated with altered mitochondrial function. Such profiles may define parameters indicative of a subject's predisposition to develop the particular disease, and may further be useful in the identification of novel subtypes of that disease. In another embodiment, correlation of one or more traits in a subject with at least one mitochondrial protein or peptide (e.g., expression levels of a mitochondrial protein that can be determined to differ from a control in a statistically significant manner) may be used to gauge the subject's responsiveness to, or the efficacy of, a particular therapeutic treatment. Similarly, where levels of at least one indicator mitochondrial protein or peptide and risk for a particular disease associated with altered mitochondrial function are correlated, the present invention provides advantageous methods for identifying agents suitable for treating such disease(s), where such agents affect levels of at least one mitochondrial protein or peptide (or levels of a modification) in a biological source. Such suitable agents will be those that alter (e.g., increase or decrease) the level of at least one mitochondrial protein or peptide in a statistically significant manner. In certain preferred embodiments, a suitable agent alters a mitochondrial protein or peptide level in a manner that confers a clinical benefit, and in certain other, non-exclusive preferred embodiments, a suitable agent alters a mitochondrial protein or peptide level by causing it to return to a level detected in control or normal (e.g., disease-free) subjects.

As described herein, determination of levels of at least one mitochondrial protein or peptide may also be used to stratify a patient population (i.e., a population classified as having one or more diseases associated with altered mitochondrial function, for example, by oxidative modification of a protein). Accordingly, in another preferred embodiment of the invention, determination of levels of a mitochondrial protein or peptide in at least one protein or peptide in a biological sample from an oxidatively stressed subject may provide a useful correlative indicator for that subject. A subject so classified on the basis of mitochondrial protein expression levels may be monitored using any known clinical parameters for a specific disease referred to above, such that correlation between levels of at least one mitochondrial protein or peptide and any particular clinical score used to evaluate a particular disease may be monitored. For example, stratification of an AD patient population according to levels of at least one mitochondrial protein or peptide may provide a useful marker with which to correlate the efficacy of any candidate therapeutic agent being used in AD subjects.

In certain other embodiments, the invention provides a method of treating a patient having a disease associated with altered mitochondrial function by administering to the patient an agent that that compensates for at least one biological activity of a polypeptide that exhibits altered biological activity which accompanies the disease, wherein the polypeptide is (i) a polypeptide having an amino acid sequence as set forth in any one of SEQ ID NOS 1-3025, or (ii) a modified polypeptide that comprises at least one modification to a polypeptide having an amino acid sequence as set forth in any one of SEQ ID NOS 1-3025. As known to the art, an agent that “compensates” for an altered biological activity of a polypeptide includes an agent that counterbalances any structural or functional defect or alteration in such polypeptide, such as an altered biological activity arising as the result of a modification as provided herein, where such counterbalancing may be partial or full restoration of normal activity, or restoration to supranormal levels, so long as an effect is demonstrable in a statistically significant manner. In certain preferred embodiments the agent substantially restores at least one mitochondrial protein or peptide to a level found in control or normal subjects (which in some cases may be an undetectable level). In a most preferred embodiment, an agent that substantially restores (e.g., increases or decreases) at least one mitochondrial protein or peptide to a normal level effects the return of the level of that indicator to a level found in control subjects. In another preferred embodiment, the agent that substantially restores such an indicator confers a clinically beneficial effect on the subject. In another embodiment, the agent that substantially restores the indicator promotes a statistically significant change in the level of at least one mitochondrial protein or peptide. As noted herein, those having ordinary skill in the art can readily determine whether a change in the level of a particular mitochondrial protein or peptide brings that level closer to a normal value and/or clinically benefits the subject, based on the present disclosure. Thus, an agent that substantially restores at least one mitochondrial protein or peptide to a normal level may include an agent capable of fully or partially restoring such level. These and related advantages will be appreciated by those familiar with the art.

Any of the agents for treating a disease associated with altered mitochondrial function (e.g., oxidative modification of a protein), identified as described herein, are preferably part of a pharmaceutical composition when used in the methods of the present invention. The pharmaceutical composition will include at least one of a pharmaceutically acceptable carrier, diluent or excipient, in addition to one or more agents for treating a disease associated with oxidative modification of a protein, and, optionally, other components.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more agents for treating a disease associated with oxidative modification of a protein may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, intrathecal and intranasal. The term parenteral as used herein includes subcutaneous injections, intravenous, intramuscular, intrasternal, intracavernous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more agents for treating a disease associated with oxidative modification of a protein, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic Physiological saline is a preferred adjuvant. An injectable pharmaceutical composition is preferably sterile.

A liquid composition intended for either parenteral or oral administration should contain an amount of agent(s) for treating a disease associated with oxidative modification of a protein such that a suitable dosage will be obtained. Typically, this amount is at least 0.01 wt % of an agent for treating a disease associated with oxidative modification of a protein in the composition. When intended for oral administration, this amount may be varied to be between 0.1 and about 70% of the weight of the composition. Preferred oral compositions contain between about 4% and about 50% of the agent for treating a disease associated with oxidative modification of a protein. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the agent(s) for treating a disease associated with oxidative modification of a protein of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

In the methods of the invention, the agent(s) for treating a disease associated with oxidative modification of a protein may be administered through use of insert(s), bead(s), timed-release formulation(s), patch(es) or fast-release formulation(s).

It will be evident to those of ordinary skill in the art that the optimal dosage of the agent(s) for treating a disease associated with oxidative modification of a protein may depend on the weight and physical condition of the patient; on the severity and longevity of the physical condition being treated; on the particular form of the active ingredient, the manner of administration and the composition employed. It is to be understood that use of an agent for treating a disease associated with oxidative modification of a protein in a chemotherapy can involve such a compound being bound to an agent, for example, a monoclonal or polyclonal antibody, a protein or a liposome, which assist the delivery of said compound.

These and related advantages will be appreciated by those familiar with the art. The following Examples are offered by way of illustration and not limitation.

### EXAMPLES

### Example 1

### PREPARATION OF HUMAN HEART MITOCHONDRIA

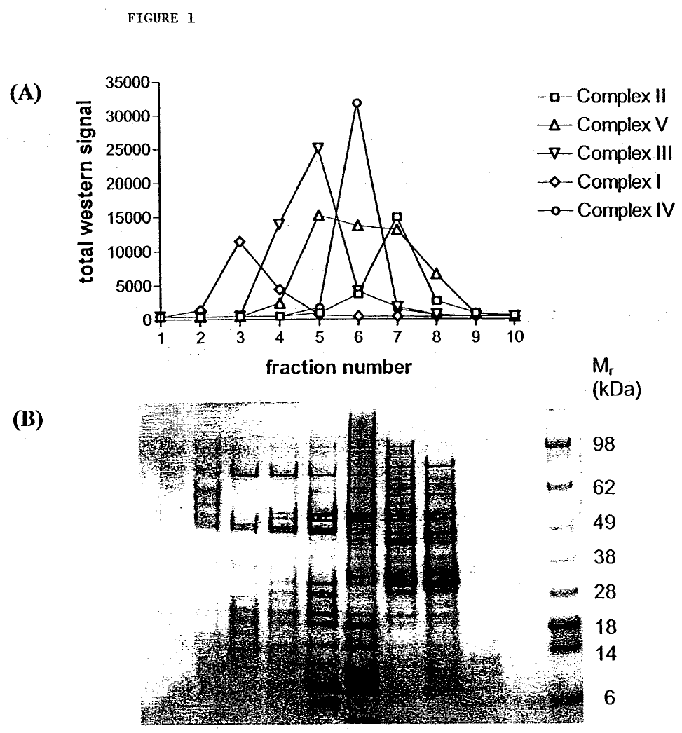

Human heart mitochondria were obtained from Analytical Biological Services (Wilmington, Del.) and were further purified by metrizamide gradient centrifugation (see, e.g., Rosenthal, R. E., et al., 1987, J. Cereb. Blood Flow Metab. 7:752-8). Mitochondria (40 mg) were resuspended in MSHE (210 mM mannitol, 70 mM sucrose, 5 mM Hepes, 1 mM EGTA plus a Complete protease inhibitor cocktail tablet (Roche, Indianapolis, Ind.)) and loaded onto a 35%/17% metrizamide gradient in 6% Percoll. Gradients were centrifuged for 45 min at 19000 rpm, 4° C. in a SW40 rotor. The heavy mitochondrial fraction was collected from the 35/17% interface, diluted in MSHE before pelleting at 12000 g for 10 min, and resuspended in MSHE. Protein concentrations were determined using the BioRad DC protein assay (BioRad Laboratories, Hercules, Calif.). The purity of the mitochondria was assessed by Western analysis using antisera directed against actin (Abcam, Cambridge, UK), dynamin II (Transduction Labs, Lexington, Ky.), KDEL, and LAMP1 (Stressgen, Victoria, BC Canada) to detect contamination due to cytoplasm, plasma membrane, ER, and lysosomes, respectively. The integrity of the mitochondria was assessed by Western analysis using a cocktail of antibodies directed against components of the electron transport chain; NDUFS2, 70 kD subunit of complex II, core I of complex III, cox 4, and ATP synthase alpha; all from Molecular Probes (Eugene, Oreg.). A representative example of western immunoblot analysis of mitochondrial fractions prepared essentially as described here is shown in FIG. 1.

### Example 2

### SUCROSE DENSITY GRADIENT FRACTIONATION OF SOLUBILIZED MITOCHONDRIA

Metrizamide purified mitochondria (13 mg) were resuspended in MSHE plus protease inhibitors and solubilized with 1% lauryl maltoside for 25 min on ice with frequent vortexing. Samples were centrifuged at 14000 rpm, 4° C. for 20 min. The pellet was frozen by immersion in liquid nitrogen and stored at −80° C. The supernatant was subjected to sucrose gradient centrifugation (Hanson, B. J. et al., 2001, Electrophoresis 22:950-959). The gradient consisted of 1 mL step-fractions of 35, 32.5, 30, 27.5, 25, 22.5, 20, 17.5, 15 and 10% sucrose in 10 mM Tris, pH 7.5/1 mM EDTA/0.05% lauryl maltoside, plus protease inhibitors). The solubilized mitochondria were loaded onto the gradient in 5% sucrose and centrifuged at 38000 rpm, 4° C. for 16.5 h in a SW40 rotor. The gradient was collected from the bottom in 1 mL fractions. The gradient fractions were concentrated in Microcon YM-3 centrifugal concentrators (Millipore, Bedford, Mass.). The concentrated samples were quantitated using the BioRad DC protein reagent, snap frozen by immersion in liquid nitrogen and stored at −80° C. Separation of proteins across the gradient was initially assessed by subjecting 1 □L aliquots of the concentrated fractions to electrophoresis on precast 4-12% NuPAGE gels in Mes buffer (Invitrogen, Carlsbad, Calif.) followed by staining with SimplyBlue Safe Stain (Invitrogen) or Western analysis using the cocktail of antibodies directed against components of the electron transport chain. Quantification of the electron transport chain complexes across the gradient was performed on images captured on a Fluor-S Multilmager (BioRad, Hercules, Calif.) and analyzed using QuantityOne software (BioRad).

Immediately prior to processing and analysis by mass spectrometry (see below), the concentrated gradient fractions and the solubilized pellet were successively subjected to electrophoresis on NuPAGE gels using ultraclean reagents. Buffers were made using HPLC grade water, and a gel rig and staining box were set aside for these samples. Aliquots (25 μg) of each concentrated gradient fraction were loaded on a 4-12% NuPage gel and run at 25 mA for 1 h, then 35 mA for another 1 h 20 min. Gels were fixed for 10 min (40% methanol, 10% acetic acid), washed three times for 5 min in HPLC grade water, stained with colloidal Coomassie for 10-15 sec, and then partially destained in water.

### Example 3

### GEL PROCESSING AND MASS SPECTROMETRIC ANALYSIS OF POLYPEPTIDES

The lightly Coomassie-stained electrophoretic gels from Example 2 were imaged placed on a light box in a laminar flow hood on a plastic cutting mat with a 65×1 mm grid placed underneath. To avoid keratin contamination all manipulations were performed wearing latex gloves, shower caps and lab coats. Starting at the bottom the gel, approximately 1 mm slices were excised across the entire width of a gel lane with a clean razor, further cut into approximately 1 mm cubes and transferred to 500 μL microcentrifuge tubes that had been prewashed with 50:50 water:acetonitrile. This procedure was progressively continued to the top the gel to ensure comprehensive coverage of all proteins in the gel lane. Although most gel slices were 1 mm thick, when discrete bands were encountered they were selectively excised, while near the top of the gel slightly thicker slices were taken where the protein concentration was lower. This resulted in 50-64 slices for each of the 12 lanes processed (corresponding to sucrose fractions 1-10, combined 11/12 and the pellet).

The gel pieces were incubated with 200 μL destain solution (25 mM ammonium bicarbonate, 25% acetonitrile) at 37° C. for 45 min. The destain solution was decanted and another cycle of destaining performed if there was residual coloration. The gel pieces were then dried on a Genevac concentrator using the “cool heat” setting (about 30 min). The dried gel pieces were slightly moistened with 5 μL 50 mM ammonium bicarbonate, 5% acetonitrile and 5 μL of freshly prepared ice cold Promega modified trypsin (0.1 mg/mL in 50 mM ammonium bicarbonate, 5% acetonitrile) added. The gel pieces were allowed to soak up the trypsin solution for 10 min, and then were fully reswelled with a 65 μL aliquot of 50 mM ammonium bicarbonate, 5% acetonitrile. After an overnight incubation at 37° C., the digestion was terminated by addition of 7.5 μL 10% acetic acid followed by brief vortexing and light centrifugation in a microcentrifuge. The digest supernatants were subsequently transferred to secondary prewashed 500 μL microcentrifuge tubes and carefully concentrated using the Genevac to final volumes of 10-20 μL. At no stage were the digests taken to dryness, in order to avoid irreversible adsorption of low abundance peptides to the walls of the tubes.

The concentrated digests were then carefully decanted to avoid particulates and transferred to the wells of a V-bottom 220 μL polypropylene microtiter 96 well plate. This plate was directly placed in a Symbiot (Applied Biosystems, Foster City, Calif.) robotic MALDI target spotter and 0.5 μL aliquots were spotted on a 2×96 well PS1 MALDI target along with a 0.3 μL aliquot of alpha-hydroxycinnamic acid matrix in 50% ACN, 0.1% TFA. Between each row of sample spots, calibrant (Des Arg1 Bradykinin, Mᵣ 904.4681; angiotensin 1, 1296.6853; Glul-Fibrinopeptide B, 1570.6774; Neurotensin, 1672.9175) was spotted for close external calibration between each successive MALDI spectrum.

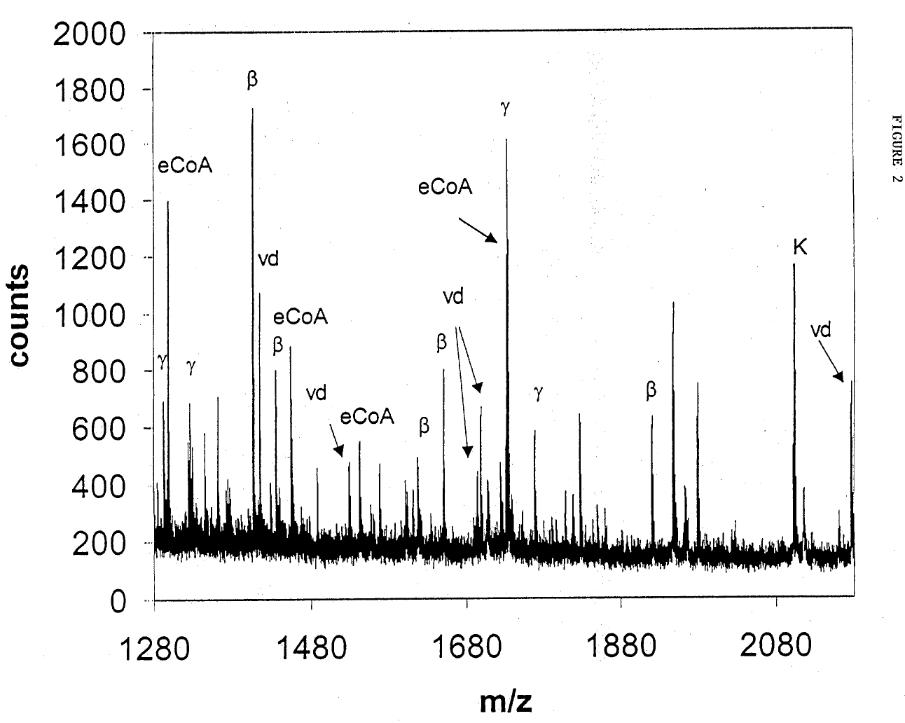

MALDI spectra were acquired on a Voyager DE-STR under the following conditions: positive reflectron mode with delayed extraction, accelerating voltage 20 kV, grid voltage 65%, mirror voltage ratio 1.12, extraction delay time 125 nsec and low mass gate 500 Da. Spectral acquisition was automated using a spiral search pattern with saved spectra being the average of 3 successful acquisitions from 400 laser shots at 20 Hz repetition rate in the m/z 850-3000 range with a minimum intensity of 750 counts in the m/z 1000-3000 range. Peptide mass fingerprints were analyzed using the program Protein Prospector (Clauser, K. R. et al., 1999, Analytical Chemistry 71, 14:2871). Peaks from baseline corrected, noise filtered deisotoped spectra were filtered to remove autolytic trypsin and most keratin peaks and then subjected to two modes of analysis. The first involved tolerant matching of 4 or 5 peaks to proteins in the database within a 100 ppm window. In general, proteins matching with MOWSE scores (see Pappin, D. J. C. et al., 1993, Current Biology 3: 327-332 for an explanation of MOWSE scores) in excess of 10000 were considered hits. The second analysis involved using the program “intellical” (Applied Biosystems) which demands high precision. As a first pass, 25 proteins would be selected from the database with 3 matches with in 150 ppm mass accuracy. The program would then look for a uniform deviation between the observed and calculated peptide masses and recalibrate the spectrum against the best fits. In general, a protein was considered a hit that had 4 peptides matching within 15 ppm of the recalibrated spectrum and MOWSE scores over 1000 using these more rigorous parameters. These analyses were fully automated using PS1 software (Applied Biosystems). FIG. 2 shows a representative example of a MALDI mass spectrum generated from polypeptides derived from a single one-dimensional gel slice.

As well as these selection criteria, the relative intensity of the matching peaks and the molecular weight of the identified protein relative to the band from which it was excised were also taken into account. The remaining portions of the digests were subjected to automated LC/MS/MS analysis. The microtiter plate containing the remaining peptide digest mixture were transferred to an Endurance autosampler connected to a MicroTech Ultimate LC system. The digest (10 μL) was transferred to a capillary trapping column containing C18 reversed phase resin at 20 μL/min using a third pump containing solvent A (95% water, 5% acetonitrile, 0.5% acetic acid) and washed for 3 min. A gradient of solvent A to solvent B (80% acetonitrile, 20% water, 0.5% acetic acid) 20% to 80% over 40 min was used to elute peptides through a 4.5 cm 75 μC-18 packed Picofrit column (New Objectives Inc., Woburn, Mass.) at a flow rate of 200-500 nL/min directly into the heated capillary orifice of a Finnigan LCQ Ion Trap Mass spectrometer equipped with a Finnigan dynamic nanospray source (Thermo Finnigan, San Jose, Calif.).

Mass spectra were acquired in the m/z 400-2000 range under the following conditions: positive polarity, capillary temperature 148° C., source voltage 2.4 kV, source current 80 μA, capillary voltage 29 V and tube lens offset 0 V. After one full scan MS of the column effluent was recorded, two MS/MS spectra of the most intense and second most intense MS peaks were recorded over the m/z 100-2000 range with an isolation width of 2.5 and normalized collision energy 35. Dynamic exclusion was employed to select the maximum number of unique peptide peaks from the chromatograms. After replicate MS/MS spectra were acquired for a precursor ion, the m/z value of ion was placed on an exclusion list with a ±1.5 u window for 3 min. Each chromatogram was subsequently analyzed with the program SEQUEST (Ducret et al., 1998, Protein Sci. 7: 706-719). The minimum requirement for a hit were at least 2 peptides for a particular protein having an X_{corr}>1.7 for a +1 ion, X_{corr}>2 for a +2 ion or X_{corr}>3. In all cases Δ_{corr} must be greater than 0.1.

A set of 3025 polypeptides [SEQ ID NOS:1-3025] was identified in the GENBANK database on the basis of the above-described selection criteria for hits from the mitochondrial protein preparations recovered according to the procedures detailed above. Table 1 presents the numbers [SEQ ID NOS:1-3025] corresponding to the Sequence Listing submitted herewith for all 3025 polypeptides identified herein as mitochondrial components, along with the GENBANK accession numbers for these sequences and (if known) a brief description of each protein based on its sequence characteristics and database annotation. Additional polypeptides that were identified included those having amino acid sequences as set forth in NCBI/Genbank Acc. Nos. 35655 and 1421609, and reference herein to any one of SEQ ID NOS:1-3025 may according to certain embodiments be understood to include NCBI/Genbank Acc. Nos. 35655 and 142160.

TABLE 1
HUMAN HEART MITOCHONDRIAL PROTEINS

| SEQ ID | GENBANK | |
|---|---|---|
| NO: | ACC. NO. | DESCRIPTION OF MITOCHONDRIAL PROTEINS |
| 1 | 13013 | ND 4 |
| 2 | 28590 | reading frame HSA |
| 3 | 28714 | anion transport protein |
| 4 | 30102 | type I collagen |
| 5 | 31474 | follicle stimulating hormone receptor |
| 6 | 31645 | glyceraldehyde 3-phosphate dehydrogenase |
| 7 | 31746 | glutathione-insulin transhydrogenase (216 AA) |
| 8 | 34670 | hexokinase 1 |
| 9 | 34719 | myeloperoxidase |
| 10 | 72146 | vitronectin precursor - human |
| 11 | 72222 | heat shock protein 90-beta - human |
| 12 | 86754 | carrier ANT3 - human (fragment) |
| 13 | 87528 | dnaK-type molecular chaperone HSPA5 precursor - human |
| 14 | 88512 | protein-L-isoaspartate(D-aspartate) O-methyltransferase (EC 2.1.1.77) |
| | | splice form II - human |
| 15 | 88650 | succinate dehydrogenase (ubiquinone) (EC 1.3.5.1) 27K iron-sulfur |
| | | protein precursor, mitochondrial - human (fragment) |
| 16 | 88741 | T-cell receptor beta chain V region - human (fragment) |
| 17 | 88972 | undulin 1 |
| 18 | 105294 | alternative splicing factor ASF-2 |
| 19 | 105475 | myosin-binding protein C, skeletal muscle - human |
| 20 | 105595 | cell adhesion protein SQM1 |
| 21 | 106140 | glycophorin A |
| 22 | 106185 | GTP-binding protein Rab2 |
| 23 | 106906 | lipopolysaccharide-binding protein |
| 24 | 106970 | mcf2 protein |
| 25 | 107554 | pyruvate kinase isozyme M2 |
| 26 | 107631 | ryanodine receptor type 1, skeletal muscle - human |
| 27 | 107912 | transcription factor E3 |
| 28 | 113962 | annexin VI |
| 29 | 114312 | Sarcoplasmic/endoplasmic reticulum calcium ATPase 2 (Calcium pump 2) |
| | | (SERCA2) (SR Ca(2+)-ATPase 2) (Calcium-transporting ATPase |
| | | sarcoplasmic reticulum type, slow twitch skeletal muscle isoform) |
| | | (Endoplasmic reticulum class 1/2 Ca(2+) ATPase) |
| 30 | 114374 | Na, K-ATPase subunit alpha 1 |
| 31 | 114374 | Sodium/potassium-transporting ATPase alpha-1 chain precursor (Sodium |
| | | pump 1) (Na+/K+ ATPase 1) |
| 32 | 114549 | ATPase beta F1 |
| 33 | 115206 | C-1-TETRAHYDROFOLATE SYNTHASE, CYTOPLASMIC (C1-THF |
| | | SYNTHASE) |
| 34 | 117103 | cox 5b |
| 35 | 117759 | UCR 4 CYTOCHROME C1 |
| 36 | 117863 | UCR cyt b |
| 37 | 120643 | GLYCERALDEHYDE 3-PHOSPHATE DEHYDROGENASE, MUSCLE |
| 38 | 120749 | MAJOR GASTROINTESTINAL TUMOR-ASSOCIATED PROTEIN |
| | | GA733-2 |
| 39 | 121665 | Glutathione peroxidase 1 (GSHPx-1) (Cellular glutathione peroxidase) |
| 40 | 123277 | HOMEOBOX PROTEIN HOX-C6(HHO.C8) |
| 41 | 123571 | heat shock 27 KD protein |
| 42 | 123678 | heat shock 90 kD protein HSP 90-ALPHA (HSP 86) |
| 43 | 123678 | Heat shock protein HSP 90-alpha (HSP 86) |
| 44 | 125484 | HEPATOCYTE GROWTH FACTOR RECEPTOR PRECURSOR(C- |
| | | MET)(HGF-SF RECEPTOR) |
| 45 | 129070 | pyruvate dehydrogenase E 1-beta |
| 46 | 129379 | heat shock 60 kDa protein, mitochondrial precursor (Hsp60) (60 kDa |
| | | chaperonin) (CPN60) (Heat shock protein 60) (HSP-60) (Mitochondrial |
| | | matrix protein P1) (P60 lymphocyte protein) (HuCHA60) |
| 47 | 129902 | Phosphoglycerate kinase 1 (Primer recognition protein 2) (PRP 2) |
| 48 | 130749 | ALKALINE PHOSPHATASE, TISSUE-NONSPECIFIC ISOZYME |
| | | PRECURSOR |
| 49 | 132164 | RETINOBLASTOMA-ASSOCIATED PROTEIN(P105-RB) |
| 50 | 136066 | TRIOSEPHOSPHATE ISOMERASE |
| 51 | 136090 | TROPOMYOSIN BETA CHAIN, SKELETAL MUSCLE |
| 52 | 136213 | Troponin I, cardiac muscle |
| 53 | 141686 | ZINC FINGER PROTEIN 8 |
| 54 | 177836 | alpha-1-antitrypsin precursor |
| 55 | 178345 | alloalbumin Venezia |
| 56 | 178390 | aldehyde dehydrogenase |
| 57 | 178426 | alpha-fodrin |
| 58 | 178736 | apolipoprotein B100 |
| 59 | 178896 | beta-3-adrenergic receptor |
| 60 | 179279 | ATPase beta subunit |
| 61 | 180529 | chromogranin A |
| 62 | 181238 | cytochrome c1 |
| 63 | 184477 | retinoic acid receptor |
| 64 | 188590 | myosin light chain 3 |
| 65 | 188672 | mannose 6-phosphate receptor |
| 66 | 189422 | proliferating cell nuclear protein P120 |
| 67 | 189514 | p80-coilin |
| 68 | 190201 | porin |
| 69 | 190474 | salivary proline-rich protein 1 |
| 70 | 190804 | ubiquinone-binding protein |
| 71 | 190804 | UCR 6 ubiquinone-binding protein |
| 72 | 223374 | isomerase, triosephosphate |
| 73 | 223582 | histone H4 |
| 74 | 223632 | dismutase, Cu/Zn superoxide |
| 75 | 224309 | protein delta T3, glyco |
| 76 | 225897 | glycogen phosphorylase |
| 77 | 225985 | amyloid related serum protein SAA |
| 78 | 226007 | ventricular myosin L1 |
| 79 | 226021 | growth regulated nuclear 68 protein |
| 80 | 226209 | cox 8 |
| 81 | 227297 | ND FeS NADH dehydrogenase FeS protein |
| 82 | 227448 | phosphofructokinase |
| 83 | 228097 | receptor-like Tyr phosphatase |
| 84 | 229149 | hemoglobin beta |
| 85 | 229479 | lipoprotein Gln I |
| 86 | 229479 | lipoproteinGln I |
| 87 | 230004 | Human Neutrophil Elastase (HNE) (E.C.3.4.21.37) (Also Referred To As |
| | | Human Leucocyte Elastase (HLE)) Complex With Methoxysuccinyl-Ala- |
| | | Ala-Pro-Ala Chloromethyl Ketone (MSACK) |
| 88 | 231743 | G1/S-SPECIFIC CYCLIN D3 |
| 89 | 232472 | nucleotide diphosphate kinase subunit A, p19/nm23-H1 [human, Peptide |
| | | Partial, 12 aa, segment 1 of 3] |
| 90 | 238427 | Porin 31HM [human, skeletal muscle membranes, Peptide, 282 aa] |
| 91 | 251188 | protein phosphatase from PCR fragment H9 |
| 92 | 283950 | oxoglutarate dehydrogenase (lipoamide) (EC 1.2.4.2) precursor - human |
| 93 | 284319 | mucin-associated antigen - human (fragment) |
| 94 | 285975 | rab GDI |
| 95 | 292793 | T-cell receptor beta |
| 96 | 306926 | insulin-like growth factor binding protein 2 |
| 97 | 307021 | mu-immunoglobulin |
| 98 | 312137 | aldolase C |
| 99 | 337758 | pre-serum amyloid P component |
| 100 | 338017 | SEF2-1D protein |
| 101 | 339647 | thyroid hormone binding protein precursor |
| 102 | 346275 | myelin transcription factor 1 - human (fragment) |
| 103 | 352335 | reductase, NADH cytochrome b5 |
| 104 | 385479 | N-methyl-D-aspartate glutamate receptor channel; NMDA GluR channel |
| 105 | 386745 | guanine nucleotide-binding protein G-s-alpha-3 |
| 106 | 386872 | myoglobin |
| 107 | 387010 | pyruvate dehydrogenase E1-beta subunit precursor |
| 108 | 387011 | pyruvate dehydrogenase E1-alpha |
| 109 | 387011 | pyruvate dehydrogenase E1-alpha precursor |
| 110 | 387016 | phosphoglycerate mutase |
| 111 | 393124 | Unknown |
| 112 | 416776 | CD27 LIGAND(CD70 ANTIGEN) |
| 113 | 434755 | rat general mitochondrial matrix processing protease mRNA (RATMPP)., |
| | | similar to |
| 114 | 436222 | Unknown |
| 115 | 438650 | paired box protein |
| 116 | 448295 | TLS protein |
| 117 | 458862 | heart fatty acid binding protein; hFABP |
| 118 | 469045 | h-contactin 2 precursor |
| 119 | 476780 | Ras guanine nucleotide exchange factor son-of-sevenless (sos) 1 - |
| | | human |
| 120 | 481043 | MHC class III histocompatibility antigen HLA-B-associated protein 2 |
| | | [similarity] - human |
| 121 | 483239 | homeotic protein engrailed 2 - human |
| 122 | 499158 | acetoacetyl-CoA thiolase mitochondrial |
| 123 | 516764 | motor protein |
| 124 | 516768 | motor protein |
| 125 | 533538 | diamine oxidase, copper/topa quinone containing |
| 126 | 551604 | pregnancy-specific beta-1 glycoprotein |
| 127 | 553254 | cytochrome b5 reductase (EC 1.6.2.2) |
| 128 | 553597 | myosin heavy chain beta-subunit |
| 129 | 553734 | putative |
| 130 | 553734 | Unknown |
| 131 | 577307 | The ha3662 gene product is related to mouse glycerophosphate |
| | | dehydrogenase. |
| 132 | 595267 | gastrin-binding protein 78 kDa |
| 133 | 606609 | GBP |
| 134 | 627364 | adenovirus E1A-associated 130k protein - human |
| 135 | 627367 | desmoyokin - human (fragments) |
| 136 | 631070 | AHNAK-related protein - human (fragment) |
| 137 | 687714 | dynein heavy chain, isotype 1B |
| 138 | 703083 | cytochrome b5 |
| 139 | 704445 | ATPase subunit 8 |
| 140 | 728834 | Alu subfamily SB2 sequence contamination warning entry |
| 141 | 802150 | pancreatic peptidylglycine alpha-amidating monooxygenase; PA |
| 142 | 903598 | Krueppel-type zinc finger protein |
| 143 | 992629 | orf |
| 144 | 1000865 | This CDS feature is included to show the translation of the corresponding |
| | | V_region. Presently translation qualifiers on V_region features are illegal |
| 145 | 1001941 | dihydropyridine receptor alpha 1 subunit |
| 146 | 1033182 | Y-chromosome RNA recognition motif protein |
| 147 | 1053081 | calpastatin |
| 148 | 1065362 | Adp-Ribosylation Factor 1 Complexed With Gdp, Full Length Non- |
| | | Myristoylated |
| 149 | 1070477 | insulin receptor precursor - human |
| 150 | 1071834 | dihydrolipoamide S-succinyltransferase |
| 151 | 1082355 | epidermal autoantigen 450K (clone pE450-B) - human (fragment) |
| 152 | 1082428 | GTPase-activating protein rhoGAP |
| 153 | 1082553 | JC-kappa protein |
| 154 | 1082567 | laminin A3 |
| 155 | 1082692 | phospholipase C beta 3 |
| 156 | 1082723 | propionyl Coenzyme A carboxylase, beta polypeptide |
| 157 | 1082723 | propionyl-CoA carboxylase (EC 6.4.1.3) beta chain precursor - human |
| 158 | 1085294 | cell-cycle-dependent 350K nuclear protein - human (fragment) |
| 159 | 1085373 | protein disulfide-isomeraseER60 precursor |
| 160 | 1091688 | heat shock protein |
| 161 | 1096024 | isoAsp protein carboxyl methyltransferase |
| 162 | 1096067 | tat-associated protein |
| 163 | 1103677 | myosin-light-chain kinase |
| 164 | 1124876 | Krueppel-related DNA-binding protein |
| 165 | 1130694 | erythrocyte adducin alpha subunit |
| 166 | 1136416 | mitosis-specific chromosome segregation protein SMC1 of S. cerevisiae., |
| | | similar to |
| 167 | 1136741 | predicted protein of 548 amino acids |
| 168 | 1151113 | PDE1C3 |
| 169 | 1160932 | DRAL gene product gi\|7209525\|dbj\|BAA92253.1\| (AB038794) |
| | | DRAL/Slim3/FHL2 |
| 170 | 1168719 | C6.1A PROTEIN |
| 171 | 1168781 | EXTRACELLULAR CALCIUM-SENSING RECEPTOR PRECURSOR |
| 172 | 1169072 | APOPAIN PRECURSOR (CYSTEINE PROTEASE CPP32) (YAMA |
| | | PROTEIN) (CPP-32) (CASPASE-3) |
| 173 | 1169204 | dodecenoyl-CoA Delta-isomerase |
| 174 | 1170654 | ANTIGEN KI-67 |
| 175 | 1172554 | VDAC-2 |
| 176 | 1174572 | Thromboxane A2 receptor (TXA2-R) (Prostanoid TP receptor) |
| 177 | 1177230 | zinc finger |
| 178 | 1177438 | brca2 |
| 179 | 1184699 | tyrosyl-tRNA synthetase |
| 180 | 1196398 | Unknown |
| 181 | 1196433 | Unknown |
| 182 | 1220311 | elongation factor-1 alpha |
| 183 | 1235848 | HMG CoA synthase |
| 184 | 1235902 | FRAP-related protein |
| 185 | 1237406 | Cu/Zn-superoxide dismutase |
| 186 | 1245894 | cardiac myosin binding protein-C |
| 187 | 1245985 | beta 2-adrenergic receptor, beta 2AR {Y354A} [human, Peptide Partial |
| | | Mutagenesis, 24 aa] |
| 188 | 1246236 | ptp-IV1b, PTP-IV1 gene product |
| 189 | 1262579 | ND 1 |
| 190 | 1262580 | ND 2 |
| 191 | 1262581 | cox 1 |
| 192 | 1262582 | ATPase 6 |
| 193 | 1292941 | hydroxymethylglutaryl-CoA lyase |
| 194 | 1293561 | Diff40 gene product |
| 195 | 1335064 | fibrillin |
| 196 | 1335072 | G34 (big gastrin) |
| 197 | 1335212 | medullasin N-term. |
| 198 | 1335250 | Rod cGMP phosphodiesterase |
| 199 | 1335277 | Unknown |
| 200 | 1340142 | alpha1-antichymotrypsin |
| 201 | 1346317 | heat shock 70 kD protein 7 |
| 202 | 1351900 | NEUROBLAST DIFFERENTIATION ASSOCIATED PROTEIN |
| 203 | 1351900 | [Segment 1 of 2] Neuroblast differentiation associated protein AHNAK |
| | | (Desmoyokin) |
| 204 | 1351901 | NEUROBLAST DIFFERENTIATION ASSOCIATED PROTEIN |
| 205 | 1354222 | aldehyde dehydrogenase E3 |
| 206 | 1359715 | Na+, K+ ATPase |
| 207 | 1359715 | Na+, K+ ATPase |
| 208 | 1359759 | histamine H2 receptor |
| 209 | 1362755 | endopeptidase La homolog (EC 3.4.21.—) precursor, mitochondrial |
| | | (version 1) |
| 210 | 1381814 | skeletal muscle LIM-protein SLIM |
| 211 | 1399105 | phosphatidylinositol (4,5)bisphosphate 5-phosphatase homolog |
| 212 | 1399801 | p167 |
| 213 | 1408188 | desmin |
| 214 | 1504020 | Yeast translation activator GCN1 (P1: A48126), similar to |
| 215 | 1517899 | RAGE-1 ORF5; one of 3 possible coding regions |
| 216 | 1582692 | TATA box-binding protein |
| 217 | 1587138 | sorcin |
| 218 | 1587477 | TCOF1 gene |
| 219 | 1588292 | Ca channel: SUBUNIT = alpha: ISOTYPE = L |
| 220 | 1655594 | HES1 |
| 221 | 1657266 | S10 GTP-binding protein |
| 222 | 1665723 | RPD3 protein |
| 223 | 1688267 | polo like kinase |
| 224 | 1706611 | ELONGATION FACTOR TU, MITOCHONDRIAL PRECURSOR |
| 225 | 1708098 | Histone H1t |
| 226 | 1709123 | DNA MISMATCH REPAIR PROTEIN MSH6 (MUTS-ALPHA 160 KDA |
| | | SUBUNIT |
| 227 | 1709947 | PYRUVATE CARBOXYLASE PRECURSOR |
| 228 | 1710279 | dihyrolipoamide acetyl transferase |
| 229 | 1718502 | aconitase mitochondrial |
| 230 | 1718502 | aconitase, mitochondrial |
| 231 | 1730078 | 130 KDA LEUCINE-RICH PROTEIN(GP130) |
| 232 | 1731414 | ZINC FINGER PROTEIN 138 |
| 233 | 1762533 | carnitine palmitoyltransferase I |
| 234 | 1763238 | lysosomal trafficking regulator LYST |
| 235 | 1773381 | APXL |
| 236 | 1778410 | unknown |
| 237 | 1778432 | Treacher Collins syndrome |
| 238 | 1805280 | alpha II spectrin |
| 239 | 1869803 | fatty acid binding protein 3 |
| 240 | 1930110 | GM-CSF receptor alpha subunit soluble 3 |
| 241 | 1942187 | Lactoferrin, H253m N Terminal Lobe Of Human |
| 242 | 1943532 | Profilin I Crystallized In High Salt Actin-Binding Protein, Human Platelet |
| 243 | 2078329 | 3-hydroxyacyl-CoA dehydrogenase, isoform 2 |
| 244 | 2078470 | Putative gene. Genscan predictions confirmed by EST splicing.; coded for |
| | | by human cDNAs AA122029 (NID: g1678048), D31562 (NID: g644442), |
| | | AA158721 (NID: g1733515), R59640 (NID: g830335) and F13082 |
| | | (NID: g709111) |
| 245 | 2114493 | RNA editase |
| 246 | 2117022 | zinc finger 5 protein |
| 247 | 2117163 | leukocyte antigen, HLA-A2 variant |
| 248 | 2117707 | dihydrolipoamide S-(2-methylpropanoyl)transferase (EC 2.3.1.—) precursor - |
| | | human |
| 249 | 2117873 | pyruvate kinase (EC 2.7.1.40), muscle splice form M1 - human |
| 250 | 2118344 | arginine - tRNA ligase (EC 6.1.1.19) - human |
| 251 | 2118970 | histone H1 - human (fragment) |
| 252 | 2119268 | alpha-tubulin - human (fragment) |
| 253 | 2119390 | proapo-A-I protein - human |
| 254 | 2119533 | giantin |
| 255 | 2119712 | dnaK-type molecular chaperone HSPA1L heat shock protein |
| 256 | 2119918 | P43 - human |
| 257 | 2134903 | CG1 protein, kinectin 1 |
| 258 | 2135068 | enhancer protein |
| 259 | 2135611 | melanoma ubiquitous mutated protein - human (fragment) |
| 260 | 2135819 | neuropolypeptide h3, brain |
| 261 | 2135911 | 3′,5′-cyclic-nucleotide phosphodiesterase (EC 3.1.4.17) 4A, cAMP- |
| | | specific, long splice form - human |
| 262 | 2136207 | succinate-semialdehyde dehydrogenase (EC 1.2.1.24) - human |
| | | (fragment) |
| 263 | 2136282 | TOG protein |
| 264 | 2144337 | pyruvate dehydrogenase (lipoamide) (EC 1.2.4.1) beta chain precursor, |
| | | long splice form - human |
| 265 | 2145011 | putative collagen homolog protein-b |
| 266 | 2146960 | methyl CpG binding protein 2 - human (fragment) |
| 267 | 2217933 | PKU-beta |
| 268 | 2224581 | Unknown |
| 269 | 2224583 | Unknown |
| 270 | 2224621 | Unknown |
| 271 | 2224663 | Unknown |
| 272 | 2243110 | Unknown |
| 273 | 2244654 | HS24/P52 |
| 274 | 2270925 | beta4-integrin |
| 275 | 2286145 | caspase-like apoptosis regulatory protein |
| 276 | 2293556 | Ran binding protein 2 |
| 277 | 2306809 | X-linked nuclear protein |
| 278 | 2317769 | probable zinc finger protein H101 |
| 279 | 2393734 | C. elegans F11A10.5; 80% similarity to Z68297 (Pl |
| 280 | 2393763 | NAD (H)-specific isocitrate dehydrogenase gamma subunit |
| 281 | 2454586 | reverse transcriptase |
| 282 | 2465178 | COX7RP |
| 283 | 2498864 | RRP5 PROTEIN HOMOLOG |
| 284 | 2499753 | PROTEIN-TYROSINE PHOSPHATASE KAPPA PRECURSOR |
| 285 | 2506118 | MULTIDRUG RESISTANCE PROTEIN 1 |
| 286 | 2507187 | PROTEIN-L-ISOASPARTATE(D-ASPARTATE) O- |
| | | METHYLTRANSFERASE (PROTEIN-BETA-ASPARTATE |
| | | METHYLTRANSFERASE) (PIMT) |
| 287 | 2511440 | calcium/calmodulin-dependent protein kinase II; CaM kinase II |
| 288 | 2511779 | beta III spectrin |
| 289 | 2565032 | transcription activator/repressor protein delta/YY1; similar |
| 290 | 2624694 | Single-Stranded Dna Binding Protein, Human Mitochondrial |
| 291 | 2653817 | lipopolysaccharide binding protein |
| 292 | 2661211 | oxidative 3 alpha hydroxysteroid dehydrogenase |
| 293 | 2662397 | HADHB |
| 294 | 2665782 | voltage-gated sodium channel, subtype III |
| 295 | 2695574 | leukocyte function-associated molecule-1 alpha subunit |
| 296 | 2769254 | NIPSNAP2 protein |
| 297 | 2769254 | NIPSNAP2 protein |
| 298 | 2811135 | retinal rod Na+/Ca+, K+ exchanger |
| 299 | 2822143 | R30217_1 |
| 300 | 2852604 | Unknown |
| 301 | 2865252 | Unknown |
| 302 | 2873377 | exportin t |
| 303 | 2981731 | Cypa Complexed With Hagpia |
| 304 | 3012097 | F22329_1 |
| 305 | 3021386 | zinc finger protein |
| 306 | 3023143 | kappa 1 immunoglobulin light chain variable region |
| 307 | 3043584 | Unknown |
| 308 | 3043646 | Unknown |
| 309 | 3046880 | LIM-homeodomain protein LMX1B/LMX1.2 |
| 310 | 3114510 | T State Human Hemoglobin [alpha V96w], Alpha Aquomet, Beta Deoxy |
| 311 | 3123721 | ND 24 K NADH dehydrogenase 24-kDa subunit of complex I |
| 312 | 3153859 | thioredoxin delta 3 |
| 313 | 3168604 | proline and glutamic acid rich nuclear protein isoform |
| 314 | 3211975 | putative glialblastoma cell differentiation-related protein |
| 315 | 3211977 | sarco-/endoplasmic reticulum Ca-ATPase 3 |
| 316 | 3212539 | Isovaleryl-Coa Dehydrogenase At 2.6 Angstroms Resolution: Structural |
| | | Basis For Substrate Specificity |
| 317 | 3252827 | Unknown |
| 318 | 3252827 | Unknown |
| 319 | 3256185 | target of myb1homolog) |
| 320 | 3273228 | acyl-CoA dehydrogenase very-long-chain |
| 321 | 3273386 | plasmalemmal porin |
| 322 | 3294170 | dJ232K4.1 (hypothetical 141.7 kD protein JUMONJI) |
| 323 | 3299887 | ES/130-related protein |
| 324 | 3327040 | Unknown |
| 325 | 3327054 | Unknown |
| 326 | 3327054 | Unknown |
| 327 | 3360457 | cul-3 |
| 328 | 3402141 | Lysozymes At Constant Positions |
| 329 | 3402145 | Lysozyme |
| 330 | 3540239 | ND Fe-S2 NADH dehydrogenase-ubiquinone Fe-S protein 2 precursor |
| 331 | 3599521 | musculin |
| 332 | 3641621 | gp180-carboxypeptidase D-like enzyme |
| 333 | 3641621 | gp180-carboxypeptidase D-like enzyme |
| 334 | 3660040 | Fkbp Mutant F36v Complexed With Remodeled Synthetic Ligand |
| 335 | 3660556 | hdkk-4 |
| 336 | 3694663 | Unknown |
| 337 | 3717965 | DIA-12C |
| 338 | 3766197 | succinyl-CoA synthetase beta subunit, ATP-specific |
| 339 | 3766197 | succinyl-CoA synthetase beta subunit, ATP-specific |
| 340 | 3766199 | succinyl-CoA synthetase beta subunit GTP-specific |
| 341 | 3766451 | CHRNB2 |
| 342 | 3882147 | Unknown |
| 343 | 3882301 | Unknown |
| 344 | 3885362 | sepiapterin reductase |
| 345 | 3891975 | Cathepsin G |
| 346 | 3982589 | SOX-28 protein |
| 347 | 3986482 | translation initiation factor elF3 p40 subunit; elF3p40 |
| 348 | 4008131 | chaperonin 10 |
| 349 | 4096860 | fibronectin |
| 350 | 4097409 | PAX-9 |
| 351 | 4103446 | NAD+-specific isocitrate dehydrogenase beta subunit isoform A |
| 352 | 4127947 | guanine nucleotide-exchange factor |
| 353 | 4139720 | Chymase |
| 354 | 4151929 | PCAF-associated factor 400 |
| 355 | 4153874 | single-stranded mitochondrial DNA-binding protein precursor |
| 356 | 4204963 | MUC-1/X mucin short variant |
| 357 | 4206175 | ubiquitin-specific protease |
| 358 | 4210351 | novel protein |
| 359 | 4240227 | Unknown |
| 360 | 4240243 | Unknown |
| 361 | 4240305 | Unknown |
| 362 | 4261577 | CD8 beta chain |
| 363 | 4262430 | CMP-NeuAc: lactosylceramide alpha-2,3-sialyltransferase |
| 364 | 4263556 | Unknown |
| 365 | 4406346 | guanylate cyclase activating protein 3 |
| 366 | 4406564 | succinyl-CoA synthetase beta subunit GTP-specific |
| 367 | 4406651 | h-sco1 |
| 368 | 4416457 | mitotic checkpoint protein |
| 369 | 4495063 | yeast suppressor protein SRP40) dJ108K11.3 (similar to |
| 370 | 4501869 | acyl-Coenzyme A oxidase 2, branched chain |
| 371 | 4501967 | alpha-2C-adrenergic receptor; alpha-2C-1 adrenergic receptor; alpha-2C- |
| | | 1 adrenoceptor; alpha-2-adrenergic receptor, renal type; alpha2-AR-C4 |
| 372 | 4502011 | adenylate kinase 1 |
| 373 | 4502013 | adenylate kinase 2 isoform a; Adenylate kinase-2, mitochondrial |
| 374 | 4502097 | solute carrier family 25 (mitochondrial carrier; adenine nucleotide |
| | | translocator), member 4; adenine nucleotide translocator 1 (skeletal |
| | | muscle) |
| 375 | 4502101 | annexin I |
| 376 | 4502107 | annexin V |
| 377 | 4502111 | annexin VII isoform 1 |
| 378 | 4502201 | ADP-ribosylation factor 1 |
| 379 | 4502273 | ATPase, Na+/K+ transporting, alpha 3 polypeptide |
| 380 | 4502297 | ATPase delta F1 |
| 381 | 4502303 | ATPase OSCP F1 |
| 382 | 4502327 | AU RNA-binding protein/enoyl-Coenzyme A hydratase precursor |
| 383 | 4502331 | arginine vasopressin receptor 1A; V1a vasopressin receptor; |
| | | vascular/hepatic-type arginine vasopressin receptor; antidiuretic hormone |
| | | receptor 1A |
| 384 | 4502379 | BCL10 |
| 385 | 4502419 | biliverdin reductase B (flavin reductase (NADPH)) |
| 386 | 4502457 | ATP-binding cassette, sub-family B (MDR/TAP), member 11; ABC |
| | | member 16, MDR/TAP subfamily |
| 387 | 4502459 | basigin; collagenase stimulatory factor; M6 antigen |
| 388 | 4502509 | complement component 5 receptor 1 (C5a ligand); complement |
| | | component-5 receptor-2 (C5a ligand) |
| 389 | 4502517 | carbonic anhydrase I |
| 390 | 4502563 | calpain 2, large subunit |
| 391 | 4502601 | carbonyl reductase 3; carbonyl reductase3 [Homo sap |
| 392 | 4502603 | chromobox homolog 4 (Pc class homolog, Drosophila); chromobox |
| | | homolog 4 (Drosophila Pc class) |
| 393 | 4502703 | CDC6 homolog; CDC6 (cell division cycle 6, S. cerevisiae) homolog; |
| | | CDC18 (cell division cycle 18, S. pombe, homolog)-like; CDC6-related |
| | | protein |
| 394 | 4502719 | cadherin 13 preproprotein; H-cadherin; heart-cadherin; T-cad |
| 395 | 4502841 | carbohydratesulfotransferase 1 |
| 396 | 4502855 | sarcomeric mitochondrial creatine kinase precursor; creatine kinase, |
| | | mitochondrial 2; basic-type mitochondrial creatine kinase |
| 397 | 4502985 | cox 6b |
| 398 | 4502987 | cox 7a muscle |
| 399 | 4502989 | cox 7a liver |
| 400 | 4502991 | cox 7b |
| 401 | 4502993 | cox 7c |
| 402 | 4503015 | copine III |
| 403 | 4503021 | liver carnitine palmitoyltransferase I; L-CPT1 |
| 404 | 4503049 | cysteine-rich protein 2; Cystein-rich intestinal protein |
| 405 | 4503057 | crystallin, alpha B; crystallin, alpha-2; Rosenthal fiber component; heat- |
| | | shock 20 kD like-protein |
| 406 | 4503143 | cathepsin D |
| 407 | 4503177 | chromosome X open reading frame 2 |
| 408 | 4503269 | deoxycytidine kinase gi\|11436224\|ref\|XP_00347 |
| 409 | 4503301 | 2,4-dienoyl CoA reductase 1 precursor |
| 410 | 4503375 | dihydropyrimidinase |
| 411 | 4503431 | dysferlin; dystrophy-associated fer-1-like 1 |
| 412 | 4503443 | endothelin converting enzyme 1 |
| 413 | 4503447 | peroxisomal enoyl-coenzyme A hydratase-like protein; delta3,5-delta2,4- |
| | | dienoyl-CoA isomerase; peroxisomal enoyl-CoA hydratase 1; dienoyl-CoA |
| | | isomerase |
| 414 | 4503475 | eukaryotic translation elongation factor 1 alpha 2 |
| 415 | 4503507 | eukaryotic translation initiation factor 2, subunit 3 |
| 416 | 4503537 | eukaryotic translation initiation factor 4E binding protein 3 |
| 417 | 4503607 | electron transfer flavoprotein alpha polypeptide |
| 418 | 4503609 | electron transfer flavoprotein beta polypeptide |
| 419 | 4503613 | envoplakin |
| 420 | 4503651 | fatty-acid-Coenzyme A ligase, long-chain 1 |
| 421 | 4503667 | fibrillin 2 + F422 |
| 422 | 4503731 | FK506-binding protein 6 |
| 423 | 4503835 | frizzled 9 |
| 424 | 4503843 | adaptor-related protein complex 1, gamma 2 subunit; gamma2-a |
| 425 | 4503899 | N-acetylgalactosamine-6-sulfatase precursor |
| 426 | 4503937 | glioblastoma amplified sequence |
| 427 | 4504041 | guanine nucleotide binding protein (G protein), alpha inhibiting activity |
| | | polypeptide 2; Guanine nucleotide-binding protein (G protein), alpha- |
| | | inhibiting |
| 428 | 4504049 | guanine nucleotide binding proteintransducin alpha-chain |
| 429 | 4504067 | aspartate aminotransferase 1; glutamic-oxaloacetic transamin |
| 430 | 4504071 | platelet glycoprotein lb alpha polypeptide precursor |
| 431 | 4504169 | glutathione synthetase |
| 432 | 4504189 | glutathione transferase zeta 1 (maleylacetoacetate isomerase); |
| | | glutathione transferase Zeta 1 |
| 433 | 4504483 | hypoxanthine phosphoribosyltransferase 1 |
| 434 | 4504487 | histidine-rich calcium-binding protein precursor SARCOPLASMIC |
| | | RETICULUM |
| 435 | 4504517 | heat shock 27 kD protein 1 |
| 436 | 4504521 | heat shock 60 kD protein 1 (chaperonin) |
| 437 | 4504523 | heat shock 10 kD protein 1 (chaperonin 10) |
| 438 | 4504523 | heat shock 10 kD protein 1 (chaperonin 10) |
| 439 | 4504665 | interleukin 2 receptor, beta; Interleukin-2 receptor, beta polypeptide |
| 440 | 4504689 | IMP (inosine monophosphate) dehydrogenase 2 |
| 441 | 4504733 | insulin receptor substrate 4 |
| 442 | 4504795 | inositol 1,4,5-triphosphate receptor, type 3 |
| 443 | 4504867 | ring finger protein (C3HC4 type) 8; C3HC4-type zinc finger protein; zinc |
| | | finger protein |
| 444 | 4504975 | low density lipoprotein receptor precursor; LDLR precursor; LDL receptor |
| 445 | 4504991 | leukemia inhibitory factor (cholinergic differentiation factor); cholinergic |
| | | differentiation factor |
| 446 | 4505071 | MAP-kinase activating death domain protein |
| 447 | 4505093 | monoamine oxidase B |
| 448 | 4505093 | monoamine oxidase B |
| 449 | 4505145 | malic enzyme 2, NAD(+)-dependent, mitochondrial |
| 450 | 4505145 | malic enzyme 2, NAD(+)-dependent, mitochondrial; Malic enzyme, |
| | | mitochondrial; malic enzyme 2, mitochondrial; pyruvic-malic carboxylase; |
| | | malate dehydrogenase |
| 451 | 4505153 | MAP/ERK kinase kinase 3 |
| 452 | 4505249 | mutS homolog 3 (E. coli); mutS (E. coli) homolog 3 |
| 453 | 4505257 | moesin |
| 454 | 4505257 | moesin |
| 455 | 4505355 | ND B8 |
| 456 | 4505357 | ND 9k NDUFA4 |
| 457 | 4505359 | ND B14 |
| 458 | 4505361 | ND B12 |
| 459 | 4505363 | ND 16k, SGDH |
| 460 | 4505365 | ND B17 |
| 461 | 4505367 | ND 6k |
| 462 | 4505369 | ND 18K NADH dehydrogenase (ubiquinone) Fe-S protein 4 (18 kD) |
| | | (NADH-coenzyme Q reductase); NADH dehydrogenase (ubiquinone) Fe- |
| | | S protein 4, 18 kD (NADH-coenzyme Q; mitochondrial respiratory chain |
| | | complex I (18-KD subunit) |
| 463 | 4505371 | ND 23K NADH dehydrogenase (ubiquinone) Fe-S protein 8 (23 kD) |
| | | (NADH-coenzyme Q reductase); NADH dehydrogenase (ubiquinone) Fe- |
| | | S protein 8 (23 kD) (NADH-coenzyme Q |
| 464 | 4505375 | neogenin homolog 1 (chicken); neogenin (chicken) homolog 1 |
| 465 | 4505399 | NIPSNAP homolog 1; 4-nitrophenylphosphatase domain and non- |
| | | neuronal SNAP25-like 1 |
| 466 | 4505405 | glycoprotein (transmembrane) nmb; transmembrane glycoprotein |
| 467 | 4505591 | peroxiredoxin 1; Proliferation-associated gene A; proliferation-associated |
| | | gene A (natural killer-enhancing factor A) |
| 468 | 4505621 | prostatic binding protein; phosphatidylethanolamine binding protein |
| 469 | 4505685 | pyruvate dehydrogenase (lipoamide) alpha 1; Pyruvate dehydrogenase, |
| | | E1-alpha polypeptide-1 |
| 470 | 4505687 | pyruvate dehydrogenase (lipoamide) beta; Pyruvate dehydrogenase, E1 |
| | | beta polypeptide |
| 471 | 4505693 | pyruvate dehydrogenase kinase, isoenzyme 4 |
| 472 | 4505717 | peroxisomal biogenesis factor 11A |
| 473 | 4505773 | Prohibitin |
| 474 | 4505775 | carrier phosphate isoform B |
| 475 | 4505775 | phosphate carrier precursor isoform 1b; phosphate carrier, mitochondrial; |
| | | phosphate carrier, mitochondrial precursor |
| 476 | 4505801 | phosphoinositide-3-kinase, class 3 |
| 477 | 4505869 | phospholipase C, gamma 1 (formerly subtype 148) |
| 478 | 4505887 | phospholamban |
| 479 | 4505893 | proteolipid protein 2 |
| 480 | 4505909 | peripheral myelin protein 2; M-FABP |
| 481 | 4505911 | postmeiotic segregation 1; Postmeiotic segregation increased (S. |
| | | cerevisiae)-like 1 |
| 482 | 4505925 | putative neurotransmitter receptor |
| 483 | 4505965 | POU domain, class 4, transcription factor 3 |
| 484 | 4506077 | protein kinase C substrate 80 KD-H |
| 485 | 4506091 | mitogen-activated protein kinase 6 |
| 486 | 4506189 | proteasome (prosome, macropain) subunit, alpha type, 7 |
| 487 | 4506197 | proteasome (prosome, macropain) subunit, beta type, 3; Proteasome |
| | | subunit, beta type, 3 |
| 488 | 4506291 | protein tyrosine phosphatase, non-receptor type 2, isoform 1; T-cell |
| | | protein tyrosine phosphatase |
| 489 | 4506371 | RAB5B, member RAS oncogene family |
| 490 | 4506401 | raf proto-oncogene serine/threonine protein kinase |
| 491 | 4506413 | RAP1A, member of RAS oncogene family; RAS-related protein RAP1A |
| 492 | 4506445 | RNA binding motif protein 4 |
| 493 | 4506517 | regulator of G-protein signalling 2, 24 kD |
| 494 | 4506787 | IQ motif containing GTPase activating protein 1; rasGAP-like with IQ |
| | | motifs |
| 495 | 4506959 | TAL1 (SCL) interrupting locus; SCL interrupting locus |
| 496 | 4506975 | carrier family 12 (sodium/potassium/chloride transporters), member 2 |
| 497 | 4506977 | carrier family 12 (sodium/chloride transporters), member 3 |
| 498 | 4506997 | solute carrier family 25 (mitochondrial carrier; oxoglutarate carrier), |
| | | member 11; solute carrier family 20 (oxoglutarate carrier), member 4 |
| 499 | 4507007 | carrier family 25 (mitochondrial carrier, Aralar), member 12; calcium |
| | | binding mitochondrial carrier superfamily member Aralar |
| 500 | 4507021 | solute carrier family 4, anion exchanger, member 1 (erythrocyte |
| | | membrane protein band 3, Diego blood group) |
| 501 | 4507185 | sepiapterin reductase (7,8-dihydrobiopterin:NADP+ oxidoreductase); |
| | | Sepiapterin reductase |
| 502 | 4507215 | signal recognition particle 54 kD |
| 503 | 4507299 | sudD suppressor of bimD6 homolog (A. nidulans); human homolog of |
| | | Aspergillus nidulans sudD gene product; sudD (suppressor of bimD6, |
| | | Aspergillus nidulans) homolog |
| 504 | 4507389 | elongin A; transcription elongation factor B (SIII) |
| 505 | 4507401 | transcription factor 6-like 1 |
| 506 | 4507401 | transcription factor 6-like 1 (mitochondrial transcription factor 1-like) |
| 507 | 4507431 | thyrotrophic embryonic factor; Thyrotroph embryonic factor |
| 508 | 4507443 | transcription factor AP-2 beta (activating enhancer binding protein 2 beta); |
| | | transcription factor AP-2 beta (activating enhancer-binding protein 2 beta) |
| 509 | 4507609 | tumor necrosis factor (ligand) superfamily, member 9 |
| 510 | 4507643 | tumor protein D52-like 2; hD54 |
| 511 | 4507645 | triosephosphate isomerase 1 |
| 512 | 4507645 | triosephosphate isomerase 1 |
| 513 | 4507665 | tyrosylprotein sulfotransferase 1 |
| 514 | 4507677 | tumor rejection antigen (gp96) 1; Tumor rejection antigen-1 (gp96) |
| 515 | 4507713 | tetratricopeptide repeat domain 2 |
| 516 | 4507733 | Tu translation elongation factor, mitochondrial |
| 517 | 4507783 | ubiquitin-conjugating enzyme E2H (homologous to yeast UBC8) |
| 518 | 4507789 | ubiquitin-conjugating enzyme E2L 3 |
| 519 | 4507793 | ubiquitin-conjugating enzyme E2N |
| 520 | 4507841 | ubiquinol-cytochrome c reductase core protein I |
| 521 | 4507843 | ubiquinol-cytochrome c reductase core protein II |
| 522 | 4507853 | ubiquitin specific protease, proto-oncogene; Unph |
| 523 | 4507857 | ubiquitin specific protease 7 (herpes virus-associated) |
| 524 | 4507879 | voltage-dependent anion channel 1 |
| 525 | 4507913 | WAS protein family, member 1; WASP family Verprolin-homologous |
| | | protein; scar, dictyostelium, homology of, 1 |
| 526 | 4507953 | tyrosine 3-monooxygenase/tryptophan 5-monooxygenase activation |
| | | protein, zeta polypeptide; Tyrosine 3-monooxygenase/tryptophan 5- |
| | | monooxygenase activation |
| 527 | 4507963 | zinc finger protein homologous to Zfp37 in mouse |
| 528 | 4507979 | zinc finger protein 132 |
| 529 | 4522026 | Bassoon protein; match to PID: g3043642; similar to PID: g3413810, C- |
| | | terminus matches KIAA0559, N-terminus similar to |
| 530 | 4529887 | NG35 |
| 531 | 4557032 | lactate dehydrogenase B |
| 532 | 4557036 | microseminoprotein, beta |
| 533 | 4557044 | propionyl Coenzyme A carboxylase, beta polypeptide |
| 534 | 4557235 | acyl-CoA dehydrogenase very long chain |
| 535 | 4557247 | acylphosphatase 2, muscle type |
| 536 | 4557265 | beta-1-adrenergic receptor gi\|15298066\|ref\|XP |
| 537 | 4557305 | aldolase A protein |
| 538 | 4557311 | adenosine monophosphate deaminase 1 (isoform M) |
| 539 | 4557317 | annexin XI |
| 540 | 4557365 | Bloom syndrome protein |
| 541 | 4557403 | carnitine/acylcarnitine translocase; Carnitine-acylcarnitine translocase; |
| | | carnitine-acylcarnitine carrier; solute carrier family 25 |
| | | (carnitine/acylcarnitine translocase), member 20 |
| 542 | 4557403 | carrier carnitine-acylcarnitine translocase |
| 543 | 4557409 | cardiac calsequestrin 2 |
| 544 | 4557439 | cyclin-dependent kinase 3 |
| 545 | 4557451 | chromodomain helicase DNA binding protein 3; Mi-2a; zinc-finger |
| | | helicase (Snf2-like) |
| 546 | 4557565 | excision repair cross-complementing rodent repair deficiency, |
| | | complementation group 6 |
| 547 | 4557579 | fatty acid binding protein 4, adipocyte; A-FABP |
| 548 | 4557657 | immature colon carcinoma transcript 1 |
| 549 | 4557735 | monoamine oxidase A |
| 550 | 4557759 | myeloperoxidase |
| 551 | 4557765 | 5-methyltetrahydrofolate-homocysteine methyltransferase; 5- |
| | | methyltetrahydrofolate-homocysteine methyltransferase 1 |
| 552 | 4557767 | methylmalonyl Coenzyme A mutase precursor |
| 553 | 4557769 | mevalonate kinase |
| 554 | 4557771 | protein C, cardiac; myosin-binding protein C, cardiac |
| 555 | 4557775 | myosin light chain 2 |
| 556 | 4557817 | Succinyl CoA: 3-oxoacid CoA transferase |
| 557 | 4557817 | Succinyl CoA: 3-oxoacid CoA transferase; succinyl-CoA: 3-ketoacid-CoA |
| | | transferase precursor |
| 558 | 4557833 | Propionyl-Coenzyme A carboxylase, alpha polypeptide |
| 559 | 4557845 | ribonucleotide reductase M2 polypeptide |
| 560 | 4557867 | sulfite oxidase |
| 561 | 4557867 | sulfite oxidase, mitochondrial |
| 562 | 4557876 | ATP-binding cassette, sub-family A member 4; ATP binding cassette |
| | | transporter; ATP-binding transporter, retina-specific; rim protein |
| 563 | 4587083 | MRP5 |
| 564 | 4589504 | Unknown |
| 565 | 4589644 | Unknown |
| 566 | 4678807 | Unknown |
| 567 | 4680705 | CGI-33 protein |
| 568 | 4680721 | thyroid peroxidase |
| 569 | 4689104 | ND ASHI |
| 570 | 4730927 | spermatogenesis associated PD1 |
| 571 | 4757732 | programmed cell death 8 (apoptosis-inducing factor) |
| 572 | 4757762 | ring finger protein 14; androgen receptor associated protein |
| 573 | 4757786 | N-acylsphingosine amidohydrolase (acid ceramidase) |
| 574 | 4757852 | BCS1 (yeast homolog)-like |
| 575 | 4758024 | coilin; coilin p80 |
| 576 | 4758030 | coatomer protein complex, subunit alpha; alpha coat protein; xenin |
| 577 | 4758038 | cox 5a |
| 578 | 4758040 | cox 6c |
| 579 | 4758118 | mitochondrial ribosomal protein S29, 28S death associated protein 3; |
| 580 | 4758118 | mitochondrial ribosomal protein S29, 28S death associated protein 3; |
| 581 | 4758120 | death-associated protein 1 |
| 582 | 4758156 | diacylglycerol kinase, iota |
| 583 | 4758192 | serine/threonine kinase 17a (apoptosis-inducing) |
| 584 | 4758242 | early development regulator 2; homolog of polyhomeotic 2 |
| 585 | 4758312 | electron-transferring-flavoprotein dehydrogenase |
| 586 | 4758352 | ferredoxin 1 precursor; adrenodoxin |
| 587 | 4758490 | GTP binding protein 1 |
| 588 | 4758498 | hexose-6-phosphate dehydrogenase precursor |
| 589 | 4758504 | hydroxyacyl-Coenzyme A dehydrogenase, type II |
| 590 | 4758520 | hect domain and RLD 2 |
| 591 | 4758520 | hect domain and RLD 2 |
| 592 | 4758570 | heat shock 70 kD protein 9B (mortalin-2); heat shock 70 kD protein 9 |
| | | (mortalin); Heat-shock 70 kD protein-9 (mortalin); mot-2; mthsp75 |
| 593 | 4758582 | isocitrate dehydrogenase 3 (NAD+) gamma |
| 594 | 4758604 | interleukin enhancer binding factor 3, 90 kD; M-phase phosphoprotein 4; |
| | | nuclear factor associated with dsRNA |
| 595 | 4758664 | acetylglucosaminyltransferase-like protein |
| 596 | 4758682 | protease, serine, 15; Lon protease-like protein |
| 597 | 4758714 | microsomal glutathione S-transferase 3 |
| 598 | 4758750 | myosin IXB |
| 599 | 4758768 | ND 42k |
| 600 | 4758772 | ND B9 |
| 601 | 4758774 | ND 22k, PDSW |
| 602 | 4758776 | ND 7k |
| 603 | 4758778 | ND 8k, AGGG |
| 604 | 4758784 | ND B14.5 |
| 605 | 4758786 | ND 49k |
| 606 | 4758788 | ND 30k |
| 607 | 4758790 | ND 15k |
| 608 | 4758792 | ND 13 k-A |
| 609 | 4758818 | Notch homolog 4 (Drosophila); Notch, drosophila, homolog of, 4; Notch |
| | | (Drosophila) homolog 4 |
| 610 | 4758832 | neuregulin 2 isoform 1; neural- and thymus-derived activator for ErbB |
| | | kinases |
| 611 | 4758852 | organic cation transporter like 3 |
| 612 | 4758940 | chromosome 14 open reading frame 2; mitochondrial proteolipid 68 MP |
| | | homolog |
| 613 | 4758940 | mitochondrial proteolipid 68 MP homolog |
| 614 | 4759020 | RAB5C, member RAS oncogene family; RAB, member of RAS oncogene |
| | | family-like; RAB5C, member of RAS oncogene family |
| 615 | 4759068 | cytochrome oxidase deficient homolog 1 |
| 616 | 4759080 | succinate dehydrogenase complex, subunit A, flavoprotein precursor; |
| | | succinate dehydrogenase complex flavoprotein subunit precursor |
| 617 | 4759080 | succinate dehydrogenase, subunit A, flavoprotein (Fp) |
| 618 | 4759082 | serum deprivation response (phosphatidylserine-binding protein) |
| 619 | 4759112 | solute carrier family 16 (monocarboxylic acid transporters), member 3; |
| | | monocarboxylate transporter 3 |
| 620 | 4759144 | carrier family 9 (sodium/hydrogen exchanger), isoform 5 |
| 621 | 4759146 | slit homolog 2 (Drosophila); slit (Drosophila) homolog 2 |
| 622 | 4759160 | small nuclear ribonucleoprotein D3 polypeptide |
| 623 | 4759196 | symplekin |
| 624 | 4760549 | IDN3 |
| 625 | 4761539 | voltage-dependent calcium channel alpha 1G subunit b isoform |
| 626 | 4826643 | annexin A3 |
| 627 | 4826649 | mitochondrial ribosomal protein L49 |
| 628 | 4826649 | mitochondrial ribosomal protein L49; chromosome 11 open reading frame 4 |
| 629 | 4826655 | calbindin 1 |
| 630 | 4826661 | nuclear receptor subfamily 1, group I, member 3 |
| 631 | 4826661 | nuclear receptor subfamily 1, group I, member 3; constitutive androstane |
| | | receptor-beta; orphan nuclear hormone receptor |
| 632 | 4826772 | insulin-like growth factor binding protein, acid labile subunit |
| 633 | 4826848 | ND B13 |
| 634 | 4826850 | ND B14.5a NADH dehydrogenase (ubiquinone) 1 alpha subcomplex, 7 |
| | | (14.5 kD, B14.5a) |
| 635 | 4826852 | ND 8k |
| 636 | 4826856 | ND 75K NADH dehydrogenase (ubiquinone) Fe-S protein 1 (75 kD) |
| | | (NADH-coenzyme Q reductase); NADH dehydrogenase (ubiquinone), Fe- |
| | | S protein-1 (75 kD); NADH-ubiquinone oxidoreductase 75 kD subunit |
| | | precursor |
| 637 | 4826898 | profilin 1 |
| 638 | 4826914 | phospholipase A2, group IVB |
| 639 | 4826950 | kallikrein 7 |
| 640 | 4827065 | zinc finger protein 147 |
| 641 | 4877291 | receptor for Advanced Glycation End Products |
| 642 | 4885281 | glutamate dehydrogenase 1 |
| 643 | 4885331 | G protein-coupled receptor 42 |
| 644 | 4885389 | hydroxyacyl glutathione hydrolase; glyoxalase 2 |
| 645 | 4885389 | hydroxyacyl glutathione hydrolase; hydroxyacyl glutathione hydrolase; |
| | | glyoxalase 2; Hydroxyacyl glutathione hydrolase; glyoxalase II; |
| | | hydroxyacylglutathione hydroxylase |
| 646 | 4885401 | cytochrome c heme lyase |
| 647 | 4885533 | peptidylglycine alpha-amidating monooxygenase COOH-terminal |
| 648 | 4885553 | postmeiotic segregation increased 2-like 9 |
| 649 | 4885565 | peroxisomal acyl-CoA thioesterase |
| 650 | 4885615 | signal transducer and activator of transcription 2, 113 kD |
| 651 | 4885665 | achaete-scute complex homolog-like 2; achaete-scute complex |
| | | (Drosophila) homolog-like 2 |
| 652 | 4887552 | MUC-B1 |
| 653 | 4894370 | ND B22 |
| 654 | 4914601 | Unknown |
| 655 | 4929697 | CGI-114 protein |
| 656 | 5031609 | branched chain alpha-ketoacid dehydrogenase kinase |
| 657 | 5031631 | CD36 antigen |
| 658 | 5031691 | chromosome 21 open reading frame 33; human HES1 protein, homolog |
| | | to E. coli and zebrafish ES1 protein |
| 659 | 5031707 | glycoprotein A repetitions predominant precursor; garpin |
| 660 | 5031777 | isocitrate dehydrogenase 3 (NAD+) alpha |
| 661 | 5031777 | isocitrate dehydrogenase 3 alpha |
| 662 | 5031875 | lamin A/C |
| 663 | 5031881 | leucyl/cystinyl aminopeptidase; leucyl/cystinyl aminopeptidase |
| | | (oxytocinase) |
| 664 | 5031943 | transcription factor NSCL-1 helix-loop-helix protein |
| 665 | 5031987 | peptidylprolyl isomerase F MITOCHONDRIAL PRECURSOR( |
| 666 | 5032017 | RAD50 (S. cerevisiae) homolog |
| 667 | 5032051 | ribosomal protein S14 40S |
| 668 | 5032095 | carrier family 21 (prostaglandin transporter), member 2 |
| 669 | 5032181 | translocase of inner mitochondrial membrane Tim17b |
| 670 | 5032215 | translational inhibitor protein |
| 671 | 5051381 | FK506 binding protein 12-rapamycin associated protein 1 |
| 672 | 5059062 | pilin-like transcription factor |
| 673 | 5114261 | voltage-dependent anion channel isoform 2 |
| 674 | 5138999 | NADH-Ubiquinone reductase |
| 675 | 5174539 | malate dehydrogenase 1, NAD (soluble) |
| 676 | 5174539 | malate dehydrogenase 1, NAD (soluble); Malate dehydrogenase, soluble |
| 677 | 5174541 | malate dehydrogenase 2, NAD (mitochondrial); Malate dehydrogenase, |
| | | mitochondrial |
| 678 | 5174563 | MHC binding factor, beta |
| 679 | 5174627 | plasma glutamate carboxypeptidase; aminopeptidase |
| 680 | 5174739 | tubulin, beta, 5 |
| 681 | 5174743 | ubiquinol-cytochrome c reductase, Rieske iron-sulfur polypeptide 1 |
| 682 | 5360087 | NY-REN-6 antigen |
| 683 | 5453549 | thioredoxin peroxidase; thioredoxin peroxidase (antioxidant enzyme) |
| 684 | 5453559 | ATPase d F0 |
| 685 | 5453670 | golgi transport complex 1 (90 kD subunit); golgi transport complex 1 (90 kDa |
| | | subunit) |
| 686 | 5453750 | brain acid-soluble protein 1; neuronal tissue-enriched acidic protein |
| 687 | 5453890 | PIBF1 gene product |
| 688 | 5453902 | NIMA-interacting, 4 (parvulin) peptidyl-prolyl cis-trans isomerase EPVH |
| 689 | 5453990 | proteasome (prosome, macropain) activator subunit 1 (PA28 alpha) |
| 690 | 5454028 | related RAS viral (r-ras) oncogene homolog; Oncogene RRAS |
| 691 | 5454122 | translocase of inner mitochondrial membrane Tim23 |
| 692 | 5454148 | UNC13 |
| 693 | 5454152 | ubiquinol-cytochrome c reductase binding protein |
| 694 | 5454180 | zinc finger protein 193 |
| 695 | 5578989 | Unknown |
| 696 | 5689405 | Unknown |
| 697 | 5689555 | Unknown |
| 698 | 5701717 | UDP-N-acetylglucosamine:alpha-1,3-D-mannoside beta-1,4-N- |
| | | acetylglucosaminyltransferase IV-homologue |
| 699 | 5725250 | G7 protein |
| 700 | 5725370 | involved in chromosomal translocation |
| 701 | 5729802 | Unknown |
| 702 | 5729875 | progesterone binding protein |
| 703 | 5729877 | heat shock 70 kD protein 8; heat shock 70 kD protein 8 (HSP73); heat |
| | | shock cognate protein, 71-kDa; heat shock 70 kd protein 10 (HSC71) |
| 704 | 5729887 | IQ motif containing GTPase activating protein 2, RasGAP-related protein |
| 705 | 5729937 | metaxin 2 |
| 706 | 5729937 | metaxin 2 |
| 707 | 5729966 | MHC class I region ORF |
| 708 | 5730027 | GAP-associated tyrosine phosphoprotein p62 (Sam68) |
| 709 | 5730033 | sodium channel, voltage-gated, type X, alpha polypeptide |
| 710 | 5730110 | ubiquitin specific protease 3 gi\|10720340\|sp\|Q9Y6I4\|UBP3_HUMAN |
| | | UBIQUITIN CARBOXYL-TERMINAL HYDROLASE 3 |
| 711 | 5759173 | succinate dehydrogenase flavoprotein subunit |
| 712 | 5802182 | PPAR gamma coactivator-1 |
| 713 | 5802814 | Gag-Pro-Pol-Env protein |
| 714 | 5802970 | AFG3 (ATPase family gene 3, yeast)-like 2 |
| 715 | 5803115 | mitofilin inner membrane protein, mitochondrial (mitofilin); motor protein |
| 716 | 5803135 | RAB35, member RAS oncogene family; ras-related protein rab-1 |
| 717 | 5803149 | coated vesicle membrane protein |
| 718 | 5803159 | sex comb on midleg (Drosophila)-like 1 |
| 719 | 5803201 | transmembrane trafficking protein |
| 720 | 5803207 | U2 small nuclear RNA auxillary factor 1; U2 snRNP auxiliary factor small |
| | | subunit; splicing factor U2AF 35 kDa subunit |
| 721 | 5821952 | Rotamer Strain As A Determinant Of Protein Structural Specificity |
| 722 | 5882259 | genethonin 3 |
| 723 | 5901896 | ATPase epsilon F1 |
| 724 | 5901926 | cleavage and polyadenylation specific factor 5, 25 kD subunit |
| 725 | 5901982 | isocitrate dehydrogenase 3 (NAD+) beta |
| 726 | 5902106 | SRY (sex determining region Y)-box 20 |
| 727 | 5902110 | SRY (sex determining region Y)-box 22; SPY (sex-determining region Y)- |
| | | box 22 |
| 728 | 5924409 | tight junction protein ZO-2 isoform C |
| 729 | 6005717 | ATPase e F0 |
| 730 | 6005772 | putative G protein coupled receptor |
| 731 | 6005938 | utrophin; dystrophin-related protein |
| 732 | 6005938 | utrophin; dystrophin-related protein |
| 733 | 6005948 | WW domain-containing binding protein 4; formin binding protein 21 |
| 734 | 6010711 | hereditary haemochromatosis protein precursor |
| 735 | 6031192 | phosphate carrier precursor isoform 1a; phosphate carrier, mitochondrial; |
| | | phosphate carrier, mitochondrial precursor |
| 736 | 6041669 | ND B15 |
| 737 | 6094658 | truncated form of cytochrome Bc1 J chain; similar to 1BGY |
| 738 | 6175038 | Son of sevenless protein homolog 2 (SOS-2) |
| 739 | 6176530 | alanine-glyoxylate aminotransferase homolog |
| 740 | 6249687 | R31155_1 |
| 741 | 6273778 | trabeculin-alpha |
| 742 | 6274550 | ND B22 NADH dehydrogenase (ubiquinone) 1 beta subcomplex, 9 (22 kD, |
| | | B22) |
| 743 | 6288790 | beta-ureidopropionase |
| 744 | 6330385 | Unknown |
| 745 | 6331429 | Unknown |
| 746 | 6382058 | v-abl Abelson murine leukemia viral oncogene homolog 1 isoform b; |
| | | Abelson murine leukemia viral (v-abl) oncogene homolog 1 |
| 747 | 6382071 | diaphanous 2 isoform 12C; Diaphanous, Drosophila, homolog of, 2; |
| | | diaphanous (Drosophila, homolog) 2 |
| 748 | 6433936 | aczonin |
| 749 | 6456828 | phosphoglycerate kinase 1 |
| 750 | 6523797 | adrenal gland protein AD-002 |
| 751 | 6572219 | UCR ubiquinol-cytochrome c reductase, Rieske iron-sulfur polypeptide- |
| | | like 1) dJ370M22.2 ( |
| 752 | 6580492 | cN28H9.1 (novel protein) |
| 753 | 6594629 | pRGR2 |
| 754 | 6598323 | GDP dissociation inhibitor 2; rab GDP-dissociation inhibitor, beta |
| 755 | 6624122 | 3-hydroxyisobutyrate dehydrogenase |
| 756 | 6631100 | natural killer-tumor recognition sequence |
| 757 | 6649914 | growth/differentiation factor-11 |
| 758 | 6678455 | transcription termination factor, RNA polymerase I |
| 759 | 6681764 | ND 39k NADH dehydrogenase (ubiquinone) 1 alpha subcomplex, 9 |
| | | (39 kD); NADH dehydrogenase (ubiquinone) Fe-S protein 2-like (NADH- |
| | | coenzyme Q reductase) |
| 760 | 6683124 | Unknown |
| 761 | 6686262 | ZINC FINGER PROTEIN 36 |
| 762 | 6688130 | poly-(ADP-ribose) polymerase II |
| 763 | 6729803 | Heat-Shock 70 kd Protein 42 kd Atpase N-Terminal Domain |
| 764 | 6739500 | LDLR-FUT fusion protein |
| 765 | 6841066 | calcium-binding transporter |
| 766 | 6841110 | Unknown |
| 767 | 6841194 | HSPC272 |
| 768 | 6841440 | HSPC108 |
| 769 | 6841930 | T cell receptor beta chain |
| 770 | 6912238 | peroxiredoxin 5; antioxidant enzyme B166 |
| 771 | 6912322 | crumbs homolog 1; crumbs (Drosophila) homolog 1 |
| 772 | 6912396 | glyoxylate reductase/hydroxypyruvate reductase |
| 773 | 6912440 | double-stranded RNA-binding zinc finger protein JAZ |
| 774 | 6912482 | LETM1 leucine zipper-EF-hand containing transmembrane protein 1 |
| 775 | 6912482 | leucine zipper-EF-hand containing transmembrane protein 1 |
| 776 | 6912536 | nicotinamide nucleotide transhydrogenase |
| 777 | 6912536 | nicotinamide nucleotide transhydrogenase |
| 778 | 6912538 | neurotensin receptor 2; neurotensin receptor, type 2 |
| 779 | 6912664 | sirtuin 5, isoform 1; sir2-like 5; sirtuin type 5; sirtuin (silent mating type |
| | | information regulation 2, S. cerevisiae, homolog) 5; silent mating type |
| | | information regulation 2, S. cerevisiae, homolog 5 |
| 780 | 6912714 | translocase of inner mitochondrial membrane 9 homolog (yeast); |
| | | translocase of inner mitochondrial membrane 9 (yeast) homolog |
| 781 | 6912714 | translocase of inner mitochondrial membrane Tim9a |
| 782 | 6996429 | acetyl-coenzyme A synthethase (acetate-coA ligase)) dJ568C11.3 (novel |
| | | AMP-binding enzyme similar to |
| 783 | 6996429 | novel AMP-binding enzyme similar to acetyl-coenzyme A synthethase |
| | | (acetate-coA ligase) |
| 784 | 7018398 | hemopoietic cell kinase |
| 785 | 7019351 | cardiotrophin-like cytokine; neurotrophin-1/B-cell stimulating factor-3 |
| 786 | 7019545 | secreted protein of unknown function |
| 787 | 7020216 | Unknown |
| 788 | 7020807 | mitochondrial ribosomal protein L22, similar to |
| 789 | 7022241 | Unknown |
| 790 | 7022343 | Unknown |
| 791 | 7022728 | Unknown |
| 792 | 7022751 | Unknown |
| 793 | 7242949 | Unknown |
| 794 | 7242979 | Unknown |
| 795 | 7243141 | Unknown |
| 796 | 7243219 | Unknown |
| 797 | 7243272 | Unknown |
| 798 | 7243280 | Unknown |
| 799 | 7245352 | Hexokinase I With Glucose And Adp In The Active Site, Mutant Monomer |
| | | Of Recombinant Human |
| 800 | 7329718 | Unknown |
| 801 | 7430427 | ionizing radiation resistance conferring protein - human |
| 802 | 7431153 | malate dehydrogenase (EC 1.1.1.37), cytosolic - human |
| 803 | 7431833 | NAD(P)+ transhydrogenase (B-specific) (EC 1.6.1.1) precursor, |
| | | mitochondrial - human |
| 804 | 7436377 | plasma membrane Ca2+-ATPase variant 4a PMCA4a - human |
| | | (fragment) |
| 805 | 7439346 | protein-tyrosine-phosphatase |
| 806 | 7441369 | tubulin beta chain - human |
| 807 | 7447071 | syntaxin |
| 808 | 7447698 | UDP glucuronosyltransferase (EC 2.4.1.—) 1A10 precursor - human |
| 809 | 7452946 | X-like 1 protein |
| 810 | 7459551 | Unknown |
| 811 | 7487801 | Unknown |
| 812 | 7511895 | Unknown |
| 813 | 7512435 | filamin, muscle |
| 814 | 7512482 | helicase II - human |
| 815 | 7512482 | helicase II - human gi\|606833\|gb\|AAC50069.1\|(U09820) helicase II |
| 816 | 7512513 | Unknown |
| 817 | 7512598 | Unknown |
| 818 | 7512628 | Unknown |
| 819 | 7512754 | Unknown |
| 820 | 7512754 | Unknown |
| 821 | 7512776 | Unknown |
| 822 | 7512977 | Unknown |
| 823 | 7513005 | Unknown |
| 824 | 7513021 | Unknown |
| 825 | 7513022 | Unknown |
| 826 | 7513076 | Unknown |
| 827 | 7513172 | N-chimerin homolog F25965_3 - human |
| 828 | 7513177 | ND 14.1K NADH dehydrogenase (ubiquinone) (EC 1.6.5.3) 14.1K chain - |
| | | human |
| 829 | 7513178 | ND acyl carrier NADH dehydrogenase (ubiquinone) (EC 1.6.5.3) acyl |
| | | carrier chain, mitochondrial - human (fragment) |
| 830 | 7513274 | probable thyroid receptor interactor - human (fragment) |
| 831 | 7513374 | thrombospondin-p50 - human (fragment) |
| 832 | 7524346 | adenylate kinase 2 isoform b; Adenylate kinase-2, mitochondrial |
| 833 | 7527760 | Unknown |
| 834 | 7582306 | ALEX3 protein |
| 835 | 7595299 | opioid growth factor receptor |
| 836 | 7643782 | HDCMD47P |
| 837 | 7656959 | calpain 7; calpain like protease; |
| 838 | 7656999 | catenin |
| 839 | 7657039 | death receptor 6 |
| 840 | 7657050 | hypothetical protein, estradiol-induced |
| 841 | 7657257 | translocase of outer mitochondrial membrane 20 (yeast) homolog |
| 842 | 7657257 | translocase of outer mitochondrial membrane 20homolog (TOM20) |
| 843 | 7657343 | metalloprotease 1 (pitrilysin family) |
| 844 | 7657347 | mitochondrial carrier homolog 2 |
| 845 | 7657347 | mitochondrial carrier homolog 2 |
| 846 | 7657369 | ND 19k NDUFA8 |
| 847 | 7657469 | rat integral membrane glycoprotein POM121, similar to |
| 848 | 7657486 | low molecular mass ubiquinone-binding protein |
| 849 | 7657534 | spastic ataxia of Charlevoix-Saguenay |
| 850 | 7657554 | soggy-1 gene; dickkopf-like 1 (soggy) |
| 851 | 7657562 | SH3-domain binding protein 4 |
| 852 | 7657581 | solute carrier family 25, member 13 (citrin) |
| 853 | 7657615 | podocin |
| 854 | 7661602 | DKFZP564B167 protein |
| 855 | 7661602 | Unknown |
| 856 | 7661678 | RAS-related protein RAP1B; K-REV DKFZP586H0723 protein; |
| 857 | 7661720 | HIRA interacting protein 5; HIRIP5 protein; HIRA-interacting protein 5; |
| | | HIRA-interacting protein 5 |
| 858 | 7661732 | HSPC009 protein |
| 859 | 7661732 | Unknown |
| 860 | 7661800 | HSPC141 protein |
| 861 | 7661872 | leucyl-tRNA synthetase, mitochondrial |
| 862 | 7661872 | leucyl-tRNA synthetase, mitochondrial; KIAA0028 protein |
| 863 | 7661960 | Rough Deal homolog, centromere/kinetochore protein; Rough Deal |
| | | (Drosophila) homolog, centromere/kinetochore protein |
| 864 | 7661996 | Unknown |
| 865 | 7662042 | Rho guanine nucleotide exchange factor 10 |
| 866 | 7662046 | Unknown |
| 867 | 7662092 | Unknown |
| 868 | 7662168 | Unknown |
| 869 | 7662190 | Unknown |
| 870 | 7662190 | Unknown |
| 871 | 7662280 | histone deacetylase 7B isoform HDRP; histone deacetylase 7; MEF-2 |
| | | interacting transcription repressor (MITR) protein; histone deacetylase 7B |
| 872 | 7662284 | Unknown |
| 873 | 7662314 | Unknown |
| 874 | 7662452 | Unknown |
| 875 | 7662470 | neuroligin 1 |
| 876 | 7662480 | Unknown |
| 877 | 7662639 | PTD011 protein |
| 878 | 7662645 | mitochondrial ribosomal protein S18B; mitochondrial ribosomal protein |
| | | S18-2; mitochondrial 28S ribosomal protein S18-2 |
| 879 | 7662673 | translocase of outer mitochondrial membrane 70 homolog A (yeast); |
| | | translocase of outer mitochondrial membrane 70 (yeast) homolog A; |
| | | KIAA0719 gene product |
| 880 | 7662673 | translocase of outer mitochondrial membrane 70homolog A |
| 881 | 7669477 | RNA-specific adenosine deaminase B1, isoform DRABA2b; RNA editase; |
| | | human dsRNA adenosine deaminase DRADA2b |
| 882 | 7669492 | glyceraldehyde-3-phosphate dehydrogenase |
| 883 | 7669520 | neuregulin 1 isoform ndf43; heregulin, alpha (45 kD, ERBB2 p 185- |
| | | activator); glial growth factor |
| 884 | 7671629 | KRAB box containing C2H2 type zinc finger protein |
| 885 | 7671653 | Unknown |
| 886 | 7677070 | silent information regulator 2 homolog |
| 887 | 7678804 | mitochondrial isoleucine tRNA synthetase |
| 888 | 7705485 | Unknown |
| 889 | 7705501 | Unknown |
| 890 | 7705594 | CGI-10 protein |
| 891 | 7705616 | CGI-112 protein |
| 892 | 7705626 | mitochondrial ribosomal protein S16 |
| 893 | 7705626 | mitochondrial ribosomal protein S16; 28S ribosomal protein S16, |
| | | mitochondrial |
| 894 | 7705646 | CGI-150 protein |
| 895 | 7705704 | glutathione S-transferase subunit 13 homolog mitochondrial |
| 896 | 7705738 | mitochondrial ribosomal protein S7; 30S ribosomal protein S7 homolog |
| 897 | 7705797 | CGI-87 protein |
| 898 | 7705805 | mitochondrial ribosomal protein S2 |
| 899 | 7705805 | mitochondrial ribosomal protein S2 |
| 900 | 7705889 | NEU1 protein |
| 901 | 7705987 | glycolipid transfer protein |
| 902 | 7706057 | mitochondrial ribosomal protein L27 |
| 903 | 7706073 | GS15 |
| 904 | 7706117 | peptide transporter 3; likely ortholog of rat peptide/histidine transporter 2 |
| 905 | 7706121 | testicular haploid expressed gene |
| 906 | 7706146 | hBOIT for potent brain type organic ion transporter |
| 907 | 7706154 | NM23-H8 |
| 908 | 7706314 | CGI-77 protein |
| 909 | 7706349 | mitochondrial ribosomal protein S33 |
| 910 | 7706449 | fatty-acid-Coenzyme A ligase, long-chain 5; long-chain acyl-CoA |
| | | synthetase 5; long-chain fatty acid coenzyme A ligase 5; FACL5 for fatty |
| | | acid coenzyme A ligase 5 |
| 911 | 7706481 | MO25 protein |
| 912 | 7706549 | CDC2-related protein kinase 7 |
| 913 | 7710129 | LIM domain only 6 |
| 914 | 7770231 | Unknown |
| 915 | 7799988 | large-conductance calcium-activated potassium channel beta |
| 916 | 7959706 | Unknown |
| 917 | 7959889 | Unknown |
| 918 | 7959907 | PRO2472 |
| 919 | 7981263 | Unknown |
| 920 | 8051579 | adenylate kinase 3; Adenylate kinase-3, mitochondrial; GTP: AMP |
| | | phosphotransferase |
| 921 | 8131894 | mitofilin |
| 922 | 8216989 | putative cell cycle control protein |
| 923 | 8217423 | bA108L7.7 (novel protein similar to C. elegans C25A1.13 (Tr: O02220)) |
| 924 | 8394499 | ubiquitin associated protein |
| 925 | 8488995 | ND 20K NADH-ubiquinone oxidoreductase 20 kDa subunit, mitochondrial |
| | | precursor (Complex I-20 KD) (CI-20 KD) (PSST subunit) |
| 926 | 8570444 | Contains similarity to an unnamed protein from Homo sapiens |
| 927 | 8574030 | diazepam binding inhibitor (GABA receptor modulator, acyl-Coenzyme A |
| | | binding protein))) dJ1013A10.3 (related to DBI ( |
| 928 | 8574070 | NFKB1 |
| 929 | 8671846 | RNA adenosine deaminase gene, exon 15, Contains similarity to |
| 930 | 8919645 | T-cell receptor beta chain |
| 931 | 8922081 | Unknown |
| 932 | 8922081 | Unknown |
| 933 | 8922275 | Unknown |
| 934 | 8922285 | Unknown |
| 935 | 8922307 | Unknown |
| 936 | 8922420 | neuropilin and tolloid like-2 |
| 937 | 8922465 | Unknown |
| 938 | 8922511 | mitochondrial ribosomal protein S18A |
| 939 | 8922517 | Unknown |
| 940 | 8922569 | Unknown |
| 941 | 8922629 | Unknown |
| 942 | 8922665 | Unknown |
| 943 | 8922701 | putative lipid kinase |
| 944 | 8922742 | Unknown |
| 945 | 8922787 | Unknown |
| 946 | 8922804 | Unknown |
| 947 | 8922838 | Unknown |
| 948 | 8923001 | Unknown |
| 949 | 8923221 | Unknown |
| 950 | 8923291 | Unknown |
| 951 | 8923390 | Unknown |
| 952 | 8923390 | Unknown |
| 953 | 8923415 | Unknown |
| 954 | 8923417 | Unknown |
| 955 | 8923528 | Unknown |
| 956 | 8923870 | hOAT4 |
| 957 | 8923930 | uncharacterized hematopoietic stem/progenitor cells protein |
| 958 | 8923930 | uncharacterized hematopoietic stem/progenitor cells protein MDS0 |
| 959 | 8927581 | testes-specific heterogenous nuclear ribonucleoprotein G-T |
| 960 | 8928067 | Malonyl-CoA decarboxylase, mitochondrial precursor (MCD) |
| 961 | 9049352 | 3-methylcrotonyl-CoA carboxylase biotin-containing subunit |
| 962 | 9256610 | protocadherin beta 15 precursor |
| 963 | 9257242 | succinate dehydrogenase complex, subunit B, iron sulfur (Ip); iron-sulfur |
| | | subunit |
| 964 | 9296943 | Cyclin T2 |
| 965 | 9297078 | UBIQUINOL-CYTOCHROME C REDUCTASE COMPLEX 7.2 KDA |
| | | PROTEIN |
| 966 | 9367862 | Unknown |
| 967 | 9438229 | phospholipase C beta 1 |
| 968 | 9501146 | meiotic DNA transesterase/topoisomerase homolog isoform 2 |
| 969 | 9506437 | FAPP1-associated protein 1 |
| 970 | 9506611 | Unknown |
| 971 | 9506611 | Unknown |
| 972 | 9506637 | rab11-binding protein gi\|7023581\|dbj\|BAA92015.1\|(AK001978) unnamed |
| | | protein product, similar to |
| 973 | 9506697 | Unknown |
| 974 | 9506713 | nucleolar protein family A, member 1; H/ACA small nucleolar RNPs |
| | | protein 1 |
| 975 | 9506785 | homeo box (H6 family) 1 |
| 976 | 9622528 | NSAID-activated protein 1 NAG-1 |
| 977 | 9884738 | AP-2 beta transcription factor |
| 978 | 9910184 | DC13 protein |
| 979 | 9910244 | mitochondrial ribosomal protein S22; gibt protein; chromosome 3 open |
| | | reading frame 5; mitochondrial 28S ribosomal protein S22 |
| 980 | 9910280 | UDP-glucose ceramide glucosyltransferase-like 1 |
| 981 | 9910382 | mitochondrial import receptor Tom22 |
| 982 | 9910382 | mitochondrial import receptor Tom22 |
| 983 | 9911130 | protein phosphatase |
| 984 | 9930803 | A kinase (PRKA) anchor protein 7 |
| 985 | 9955433 | Unknown |
| 986 | 9966799 | disrupter of silencing 10 |
| 987 | 9966893 | CGI-203 protein |
| 988 | 10047106 | carboxypeptidase A3 |
| 989 | 10047118 | G-protein gamma-12 subunit |
| 990 | 10047120 | insulin receptor tyrosine kinase substrate |
| 991 | 10047167 | Unknown |
| 992 | 10047177 | Unknown |
| 993 | 10047183 | Unknown |
| 994 | 10047187 | Unknown |
| 995 | 10047199 | Unknown |
| 996 | 10047213 | Unknown |
| 997 | 10047231 | Unknown |
| 998 | 10047239 | Unknown |
| 999 | 10047243 | Unknown |
| 1000 | 10047247 | Unknown |
| 1001 | 10047249 | Unknown |
| 1002 | 10047277 | Sarcolemmal-associated protein |
| 1003 | 10047277 | Unknown |
| 1004 | 10047279 | Unknown |
| 1005 | 10047281 | Unknown |
| 1006 | 10047283 | Unknown |
| 1007 | 10047317 | L-periaxin |
| 1008 | 10047329 | Unknown |
| 1009 | 10047335 | zinc finger protein |
| 1010 | 10047341 | Unknown |
| 1011 | 10047341 | Unknown |
| 1012 | 10047347 | Unknown |
| 1013 | 10047361 | Unknown |
| 1014 | 10092604 | HUG1 gene |
| 1015 | 10092623 | hematopoietic PBX-interacting protein gi\|9930 |
| 1016 | 10092657 | 13 kDa differentiation-associated protein; NADH: ubiquinone |
| | | oxidoreductase |
| 1017 | 10092657 | ND B17.2 |
| 1018 | 10120604 | L-3-Hydroxyacyl-Coa Dehydrogenase Complexed With Acetoacetyl-Coa |
| | | And Nad+ |
| 1019 | 10179599 | ND NDUFS2 |
| 1020 | 10179880 | muscle-specific protein |
| 1021 | 10181206 | GABA(A) receptor-associated protein like 1 |
| 1022 | 10190653 | sphingosine-1-phosphate lyase 1 |
| 1023 | 10190692 | junctophilin 3; junctophilin type3 gi\|9886738 |
| 1024 | 10241702 | putative ZIC3 Binding protein from Xenopus laevis, similar to |
| 1025 | 10241706 | Unknown |
| 1026 | 10257409 | natural resistance-associated macrophage protein 1 |
| 1027 | 10257494 | N-ethylmaleimide-sensitive factor |
| 1028 | 10334442 | hydroxysteroid (17-beta) dehydrogenase 7 |
| 1029 | 10334443 | Unknown |
| 1030 | 10334466 | Unknown |
| 1031 | 10337605 | peroxisomal short-chain alcohol dehydrogenase |
| 1032 | 10432782 | testin |
| 1033 | 10432971 | Unknown |
| 1034 | 10433147 | poly(A) polymerase gamma; SRP RNA 3′ adenylating enzyme/pap2 |
| 1035 | 10433320 | huntingtin-associated protein |
| 1036 | 10433905 | Unknown |
| 1037 | 10433929 | Unknown |
| 1038 | 10434023 | Unknown |
| 1039 | 10434055 | Unknown |
| 1040 | 10434106 | Fanconi anemia complementation group D2 protein |
| 1041 | 10434151 | Unknown |
| 1042 | 10434167 | Unknown |
| 1043 | 10434183 | Unknown |
| 1044 | 10434243 | Unknown |
| 1045 | 10434293 | Unknown |
| 1046 | 10434345 | Unknown |
| 1047 | 10434521 | Unknown |
| 1048 | 10434757 | Unknown |
| 1049 | 10434850 | zinc finger protein 226 |
| 1050 | 10434904 | Unknown |
| 1051 | 10434988 | Unknown |
| 1052 | 10435007 | Unknown |
| 1053 | 10435244 | Unknown |
| 1054 | 10435551 | Unknown |
| 1055 | 10435767 | Unknown |
| 1056 | 10435899 | Unknown |
| 1057 | 10435947 | Unknown |
| 1058 | 10436007 | Unknown |
| 1059 | 10436258 | Unknown |
| 1060 | 10436263 | Unknown |
| 1061 | 10436325 | Unknown |
| 1062 | 10436604 | Unknown |
| 1063 | 10437144 | Smac |
| 1064 | 10437144 | Unknown |
| 1065 | 10437178 | mitochondrial ribosomal protein L1 |
| 1066 | 10437189 | Unknown |
| 1067 | 10437384 | M-phase phosphoprotein 1 |
| 1068 | 10437960 | Unknown |
| 1069 | 10437984 | Unknown |
| 1070 | 10438291 | Unknown |
| 1071 | 10438353 | McKusick-Kaufman syndrome protein |
| 1072 | 10438441 | Unknown |
| 1073 | 10438702 | Unknown |
| 1074 | 10438857 | Unknown |
| 1075 | 10438928 | mitochondrial ribosomal protein S11 |
| 1076 | 10438968 | Unknown |
| 1077 | 10439079 | Unknown |
| 1078 | 10439244 | Unknown |
| 1079 | 10439312 | Unknown |
| 1080 | 10440252 | bromodomain PHD finger transcription factor |
| 1081 | 10440347 | Unknown |
| 1082 | 10440357 | Unknown |
| 1083 | 10440367 | Unknown |
| 1084 | 10440389 | Unknown |
| 1085 | 10440402 | Unknown |
| 1086 | 10440484 | Unknown |
| 1087 | 10441879 | Unknown |
| 1088 | 10441930 | Unknown |
| 1089 | 10443472 | Rhesus blood group-associated glycoprotein (RH50A) |
| 1090 | 10503988 | Unknown |
| 1091 | 10518340 | muscleblind (Drosophila)-like |
| 1092 | 10567164 | gene amplified in squamous cell carcinoma-1 |
| 1093 | 10639097 | solute carrier family 24 (sodium/potassium/calcium exchanger), member |
| | | 3) dJ122P22.1 ( |
| 1094 | 10645199 | ADAM-TS disintegrin and metalloprotease with thrombospondin motifs-7 |
| | | preproprotein; a disintegrin-like and metalloprotease (reprolysin type) with |
| | | thrombospondin type 1 motif, 7 |
| 1095 | 10716563 | calnexin |
| 1096 | 10719935 | CELL DIVISION CYCLE 2-LIKE PROTEIN KINASE 5(CDC2-RELATED PROTEIN |
| | | KINASE 5) |
| 1097 | 10720290 | SORTING NEXIN 14 |
| 1098 | 10720297 | SYNAPTOJANIN 2 (SYNAPTIC INOSITOL-1,4,5-TRISPHOSPHATE 5- |
| | | PHOSPHATASE 2) |
| 1099 | 10720409 | Zinc finger protein 294 |
| 1100 | 10764847 | ND B18 |
| 1101 | 10798812 | MLTK-alpha |
| 1102 | 10834587 | fer-1 like protein 3 |
| 1103 | 10834762 | PNAS-102 |
| 1104 | 10834786 | PNAS-117 |
| 1105 | 10834968 | mannosidase, alpha B, lysosomal |
| 1106 | 10835000 | pancreatic lipase |
| 1107 | 10835002 | Rho GDP dissociation inhibitor (GDI) beta |
| 1108 | 10835023 | inositol 1,4,5-triphosphate receptor, type 1 |
| 1109 | 10835025 | ND 24k |
| 1110 | 10835045 | retinaldehyde dehydrogenase 2 |
| 1111 | 10835057 | N-acetyltransferase, homolog of S. cerevisiae ARD1; N-acetyltransferase |
| | | ARD1, human homolog of |
| 1112 | 10835059 | farnesyltransferase, CAAX box, beta |
| 1113 | 10835063 | nucleophosmin (nucleolar phosphoprotein B23, numatrin) |
| 1114 | 10835087 | ND 10k |
| 1115 | 10835089 | neurofilament, heavy polypeptide (200 kD); Neurofilament, heavy |
| | | polypeptide |
| 1116 | 10835109 | myotubularin related protein 3; FYVE (Fab1 YGLO23 Vsp27 EEA1 |
| | | domain) dual-specificity protein phosphatase |
| 1117 | 10835155 | tumor necrosis factor (cachectin) |
| 1118 | 10835165 | CD59 antigen p18-20 |
| 1119 | 10835173 | nitric oxide synthase 1 |
| 1120 | 10835189 | glutathione reductase |
| 1121 | 10835220 | ATPase, Ca++ transporting, fast twitch 1 |
| 1122 | 10863907 | hepatocellular carcinoma associated protein; breast cancer |
| 1123 | 10863927 | peptidylprolyl isomerase A |
| 1124 | 10863945 | ATP-dependant DNA helicase II |
| 1125 | 10863985 | G4 protein |
| 1126 | 10864011 | CGI-44 protein; sulfide dehydrogenase like (yeast) |
| 1127 | 10864043 | kidney and liver proline oxidase 1 |
| 1128 | 10864077 | calcium channel, voltage-dependent, alpha 1H subunit |
| 1129 | 10945428 | membrane-associated guanylate kinase MAGI3 |
| 1130 | 11024710 | Unknown |
| 1131 | 11024714 | ubiquitin B |
| 1132 | 11034855 | Unknown |
| 1133 | 11038674 | CD79B antigen, isoform 1 precursor; B-cell-specific glycoprotein B29 |
| 1134 | 11055998 | guanine nucleotide binding protein beta subunit 4 [Homo sapi |
| 1135 | 11056030 | protocadherin gamma subfamily A, 2, isoform 1 precursor |
| 1136 | 11066958 | mutant beta-globin |
| 1137 | 11066968 | EH domain-containing protein FKSG7 |
| 1138 | 11095436 | valosin-containing protein |
| 1139 | 11096171 | RNA polymerase III transcription initiation factor B |
| 1140 | 11121497 | Trp4-associated protein TAP1, similar to |
| 1141 | 11127695 | SYT/SSX4 fusion protein |
| 1142 | 11128019 | cytochrome c |
| 1143 | 11128031 | protocadherin gamma subfamily B, 5, isoform 1 precursor |
| 1144 | 11139093 | GrpE-like protein cochaperone |
| 1145 | 11141885 | carrier family 5 (choline transporter), member 7 |
| 1146 | 11141891 | ERGL protein |
| 1147 | 11177148 | mitochondrial ribosomal protein L12 |
| 1148 | 11177148 | mitoribosomal protein L12 |
| 1149 | 11225260 | DNA TOPOISOMERASE I |
| 1150 | 11225266 | transient receptor potential cation channel, subfamily M, member 5; |
| | | MLSN1- and TRP-related; MLSN1 and TRP-related |
| 1151 | 11245229 | ninein-Lm isoform |
| 1152 | 11252721 | glutaryl-CoA dehydrogenase |
| 1153 | 11252721 | glutaryl-CoA dehydrogenase (EC 1.3.99.7) [imported] - human |
| 1154 | 11267525 | probable RNA helicase |
| 1155 | 11275568 | mucin 5B |
| 1156 | 11275986 | glycerol-3-phosphate dehydrogenase 3 |
| 1157 | 11276083 | fatty-acid-Coenzyme A ligase, long-chain 2 |
| 1158 | 11276083 | long-chain fatty-acid-Coenzyme A ligase 2; acyl-activating enzyme; acyl- |
| | | CoA synthetase; fatty acid thiokinase (long-chain); lignoceroyl-CoA |
| | | synthase; long-chain acyl-CoA synthetase 2 |
| 1159 | 11276655 | ribosomal protein S26 [imported] - human |
| 1160 | 11276938 | villin 2 |
| 1161 | 11277141 | heat shock 90 kD protein beta |
| 1162 | 11280538 | Unknown |
| 1163 | 11280677 | Unknown |
| 1164 | 11281511 | Unknown |
| 1165 | 11321341 | MondoA |
| 1166 | 11321569 | olfactory receptor, family 3, subfamily A, member 2 |
| 1167 | 11321571 | slit homolog 3 (Drosophila); slit (Drosophila) homolog 3; slit (Drosophila) |
| | | homolog 2; slit2 |
| 1168 | 11321579 | myosin, heavy polypeptide 13, skeletal muscle; extraocular muscle |
| | | myosin heavy chain |
| 1169 | 11321581 | succinyl-CoA synthetase alpha subunit |
| 1170 | 11321583 | succinate-CoA ligase, ADP-forming, beta subunit |
| 1171 | 11321613 | epilepsy, progressive myoclonus type 2, Lafora disease (laforin) |
| 1172 | 11321615 | T-box 3 protein; T-box 3; T-box transcription factor TBX3 |
| 1173 | 11323320 | ubiquitin-conjugating enzyme E2 variant 1 (isoform 2, similar to variant 2 |
| | | (UBE2V2, MMS2) |
| 1174 | 11342570 | metalloproteinase 24 (membrane-inserted), matrix |
| 1175 | 11342672 | myosin, heavy polypeptide 3, skeletal muscle, embryonic |
| 1176 | 11345448 | lipopolysaccharide-binding protein |
| 1177 | 11345456 | fibroblast growth factor receptor-like 1 precursor |
| 1178 | 11345478 | Unknown |
| 1179 | 11345539 | novel Helicase C-terminal domain |
| 1180 | 11359874 | GTP-binding protein 2 |
| 1181 | 11359883 | Unknown |
| 1182 | 11359946 | leucine zipper-EF-hand containing transmembrane protein 1 |
| 1183 | 11359985 | Unknown |
| 1184 | 11359986 | Unknown |
| 1185 | 11360009 | Bcl-Rambo |
| 1186 | 11360009 | Unknown |
| 1187 | 11360063 | matrilin 2 precursor |
| 1188 | 11360067 | Unknown |
| 1189 | 11360079 | Unknown |
| 1190 | 11360112 | Unknown |
| 1191 | 11360155 | Unknown |
| 1192 | 11360155 | Unknown |
| 1193 | 11360156 | Unknown |
| 1194 | 11360162 | Unknown |
| 1195 | 11360185 | Unknown |
| 1196 | 11360188 | Unknown |
| 1197 | 11360228 | Unknown |
| 1198 | 11360250 | Unknown |
| 1199 | 11360251 | Unknown |
| 1200 | 11360294 | Unknown |
| 1201 | 11360310 | myosin Vlla, long form - human |
| 1202 | 11360321 | properdin |
| 1203 | 11374664 | isocitrate dehydrogenase (EC 1.1.1.42), cytosolic |
| 1204 | 11385354 | polybromo 1 |
| 1205 | 11385644 | CTCL tumor antigen se2-1 |
| 1206 | 11385664 | CTCL tumor antigen se89-1 |
| 1207 | 11386147 | prosaposin |
| 1208 | 11399466 | D-2-hydroxy-acid dehydrogenase |
| 1209 | 11415024 | diacylglycerol kinase, alpha (80 kD) |
| 1210 | 11416393 | mitochondrial ribosomal protein L22 |
| 1211 | 11416669 | nicotinamide nucleotide transhydrogenase |
| 1212 | 11417363 | low molecular mass ubiquinone-binding protein |
| 1213 | 11417363 | low molecular mass ubiquinone-binding protein |
| 1214 | 11418549 | eyes absent (Drosophila) homolog 4 |
| 1215 | 11418714 | Unknown |
| 1216 | 11419832 | phosphorylase kinase, alpha 1 |
| 1217 | 11421027 | Unknown |
| 1218 | 11422272 | ribosomal protein S6 kinase, 90 kD |
| 1219 | 11423142 | basic leucine zipper nuclear factor 1 |
| 1220 | 11423880 | alpha-SNAP |
| 1221 | 11424404 | mitochondrial ribosomal protein S23 |
| 1222 | 11424724 | neurofilament 3 |
| 1223 | 11425565 | Unknown |
| 1224 | 11425836 | low density lipoprotein receptor-related protein 3 |
| 1225 | 11427613 | Unknown |
| 1226 | 11427636 | GTPase Rab14 |
| 1227 | 11428230 | aldehyde dehydrogenase 1 family, member B1 |
| 1228 | 11429803 | Unknown |
| 1229 | 11430299 | hexokinase 1 |
| 1230 | 11431667 | multiple inositol polyphosphate phosphatase 2 |
| 1231 | 11432018 | Unknown |
| 1232 | 11432441 | Unknown |
| 1233 | 11432489 | general transcription factor IIE, polypeptide 1 (alpha subunit, 56 kD) |
| 1234 | 11433007 | peroxisomal enoyl-coenzyme A hydratase-like protein |
| 1235 | 11433596 | tryptophanyl-tRNA synthetase |
| 1236 | 11434079 | Unknown |
| 1237 | 11434447 | Unknown |
| 1238 | 11434986 | COQ6_HUMAN PUTATIVE UBIQUINONE BIOSYNTHESIS |
| | | MONOOXGENASE COQ |
| 1239 | 11435257 | Unknown |
| 1240 | 11435724 | mannosidase, beta A, lysosomal |
| 1241 | 11436135 | RAS-RELATED PROTEIN R-RAS2 |
| 1242 | 11436533 | aldehyde dehydrogenase 2 family (mitochondrial) |
| 1243 | 11436778 | inositol polyphosphate-4-phosphatase, type II, 105 kD |
| 1244 | 11437205 | Unknown |
| 1245 | 11440003 | transgelin |
| 1246 | 11441230 | skeletal muscle specific actinin, alpha 3 |
| 1247 | 11493459 | PRO2619 |
| 1248 | 11493489 | PRO2620 |
| 1249 | 11493522 | Unknown |
| 1250 | 11493552 | Unknown |
| 1251 | 11496882 | ELK4 protein, isoform b; ETS-domain protein; SRF accessory protein 1 |
| 1252 | 11497601 | metallaproteinase-disintegrin |
| 1253 | 11526149 | ATPase CF6 F0 |
| 1254 | 11526456 | frataxin |
| 1255 | 11526471 | tripartite motif protein TRIM14 isoform alpha |
| 1256 | 11526573 | heat shock cognate protein 54 |
| 1257 | 11526789 | inorganic pyrophosphatase 2 |
| 1258 | 11545761 | potassium channel, subfamily K, member 12; tandem pore domain |
| | | potassium channel THIK-2 |
| 1259 | 11545847 | basic-helix-loop-helix-PAS protein |
| 1260 | 11545863 | methylcrotonoyl-Coenzyme A carboxylase 2 |
| 1261 | 11545869 | popeye protein 2 |
| 1262 | 11545894 | RFamide-related peptide precursor |
| 1263 | 11559927 | mitochondrial ribosomal protein S14 |
| 1264 | 11596402 | MAGE-D4 |
| 1265 | 11596859 | mitochondrial ribosomal protein L17 |
| 1266 | 11602741 | complement component 8, alpha polypeptide |
| 1267 | 11602963 | heparan sulfate proteoglycan perlecan |
| 1268 | 11611734 | GREB1a |
| 1269 | 11612659 | FXYD domain-containing ion transport regulator 7 |
| 1270 | 11612670 | phospholemman, isoform b precursor; FXYD domain-containing |
| 1271 | 11640566 | hydroxyacyl-Coenzyme A dehydrogenase/3-ketoacyl-Coenzyme A |
| | | thiolase/enoyl-Coenzyme A hydratase beta |
| 1272 | 11640578 | glyoxylate reductase/hydroxypyruvate reductase |
| 1273 | 11641249 | protein kinase Njmu-R1 |
| 1274 | 11641283 | LIM homeobox protein 5 |
| 1275 | 11641413 | cell division cycle 25B, isoform 3; CDC25B |
| 1276 | 11761696 | bHLHZip transcription factor BIGMAX gamma |
| 1277 | 11863673 | guanine nucleotide binding protein (G protein), alpha stimulating activity |
| | | polypeptide 1) dJ309F20.1.5 (isoform 5 of |
| 1278 | 11890755 | RNA helicase II/Gu protein |
| 1279 | 11907570 | mutant desmin |
| 1280 | 11908171 | Fas-binding protein Daxx |
| 1281 | 11935053 | sarcolemmal associated protein 1 |
| 1282 | 11968003 | 5-azacytidine induced gene 2, similar to |
| 1283 | 11968152 | somatostatin receptor-interacting protein |
| 1284 | 11990879 | phosphoglycerate kinase 2 |
| 1285 | 11991867 | odorant receptor HOR3′beta5 |
| 1286 | 12001946 | My003 protein |
| 1287 | 12001986 | My022 protein |
| 1288 | 12001992 | brain my025 |
| 1289 | 12002038 | brain my045 protein |
| 1290 | 12002042 | brain my048 protein |
| 1291 | 12002201 | serine/threonine protein kinase PFTAIRE-1 |
| 1292 | 12003293 | organic anion transporter 2 |
| 1293 | 12005493 | NPD011 |
| 1294 | 12005918 | GRIM19 |
| 1295 | 12006049 | EF1a-like protein |
| 1296 | 12006205 | TNFIP-iso |
| 1297 | 12038977 | Unknown |
| 1298 | 12043738 | thioredoxin reductase, mitochondrial |
| 1299 | 12052810 | Unknown |
| 1300 | 12052820 | COQ7 protein; timing protein; ubiquinone biosynthesis protein |
| 1301 | 12052826 | RAB-8b protein, small GTP-binding protein |
| 1302 | 12052828 | Unknown |
| 1303 | 12052872 | Unknown |
| 1304 | 12052908 | Unknown |
| 1305 | 12052971 | methyltransferase COQ3 |
| 1306 | 12052989 | Unknown |
| 1307 | 12052991 | Unknown |
| 1308 | 12053107 | Unknown |
| 1309 | 12053245 | Unknown |
| 1310 | 12053255 | Unknown |
| 1311 | 12060822 | serologically defined breast cancer antigen NY-BR-16 |
| 1312 | 12060832 | serologically defined breast cancer antigen NY-BR-40 |
| 1313 | 12061185 | ASC-1 complex subunit P200 |
| 1314 | 12081909 | semaphorin Y |
| 1315 | 12214171 | putative small GTP-binding protein (rab1b) |
| 1316 | 12214288 | dJ402H5.2 (novel protein similar to worm and fly proteins) |
| 1317 | 12230015 | CYTOCHROME B5 OUTER MITOCHONDRIAL MEMBRANE ISOFORM |
| | | PRECURSOR |
| 1318 | 12230075 | GLYCEROL KINASE, TESTIS SPECIFIC 1 |
| 1319 | 12232373 | rab6 GTPase activating protein (GAP and centrosome-associated) |
| 1320 | 12232421 | tricarboxylate carrier protein |
| 1321 | 12232477 | Unknown |
| 1322 | 12239360 | LYST-interacting protein LIP6 |
| 1323 | 12246901 | tumor protein D52-like 2 |
| 1324 | 12248755 | mono ATP-binding cassette protein |
| 1325 | 12314005 | Unknown |
| 1326 | 12314016 | transcription factor TFIIS, similar to |
| 1327 | 12314029 | proteasome subunit 7 |
| 1328 | 12314062 | Unknown |
| 1329 | 12314123 | uncharacterized hematopoietic stem/progenitor cells protein MDS030 |
| | | (8923932) |
| 1330 | 12314190 | dJ445H2.2 (novel protein) |
| 1331 | 12314195 | Unknown |
| 1332 | 12328445 | NPAS3 |
| 1333 | 12382773 | caspase recruitment domain protein 11 |
| 1334 | 12382789 | OSBP-related protein 7; ORP7 |
| 1335 | 12383092 | Unknown |
| 1336 | 12407403 | tripartite motif protein TRIM9 isoform alpha |
| 1337 | 12408656 | calpain 1, large subunit |
| 1338 | 12597655 | kinetochore protein |
| 1339 | 12620194 | Unknown |
| 1340 | 12620246 | CD36 |
| 1341 | 12620252 | CD36 |
| 1342 | 12620871 | phosphoinositide-3-kinase gamma catalytic subunit |
| 1343 | 12621903 | cathepsin S |
| 1344 | 12643256 | pilin-like transcription factor |
| 1345 | 12643326 | CIP1-INTERACTING ZINC FINGER PROTEIN (NUCLEAR PROTEIN |
| | | NP94) |
| 1346 | 12643329 | CGI-51 |
| 1347 | 12643417 | Pyruvate dehydrogenase protein X component, mitochondrial precursor |
| | | (Dihydrolipoamide dehydrogenase-binding protein of pyruvate |
| | | dehydrogenase complex) (E3-binding protein) (E3BP) (proX) |
| 1348 | 12643637 | ADAM-TS 4 PRECURSOR (A DISINTEGRIN AND |
| | | METALLOPROTEINASE WITH THROMBOSPONDIN MOTIFS 4) |
| 1349 | 12643716 | PROTEIN TYROSINE PHOSPHATASE, NON-RECEPTOR TYPE 13 |
| 1350 | 12643796 | RETINOBLASTOMA-BINDING PROTEIN 8 |
| 1351 | 12643896 | Zinc finger protein 236 |
| 1352 | 12644018 | AF-6 PROTEIN |
| 1353 | 12644090 | T-BOX TRANSCRIPTION FACTOR TBX18 |
| 1354 | 12644310 | COATOMER BETA SUBUNIT(BETA-COP) |
| 1355 | 12644370 | Zinc finger X-linked protein ZXDB |
| 1356 | 12652715 | nucleolar GTPase |
| 1357 | 12652761 | Unknown |
| 1358 | 12652763 | Unknown |
| 1359 | 12652773 | Unknown |
| 1360 | 12652981 | glycogen synthase kinase 3 beta |
| 1361 | 12652989 | Unknown |
| 1362 | 12653017 | LRP16 protein |
| 1363 | 12653371 | phosphoglycerate mutase 1 |
| 1364 | 12653507 | aspartate transaminase 2 |
| 1365 | 12653549 | mitochondrial ribosomal protein S6 |
| 1366 | 12653687 | Unknown |
| 1367 | 12653775 | helicase-like protein NHL |
| 1368 | 12653827 | mitochondrial carrier homolog 1 or presenilin-associated protein |
| 1369 | 12653855 | dynamitin |
| 1370 | 12654077 | NICE-5 protein |
| 1371 | 12654149 | Unknown |
| 1372 | 12654285 | peptide N-glycanase homolog |
| 1373 | 12654289 | transcription termination factor, mitochondrial |
| 1374 | 12654333 | HCDI protein |
| 1375 | 12654407 | N-Acetylglucosamine kinase |
| 1376 | 12654521 | Unknown |
| 1377 | 12654627 | metalloprotease 1 |
| 1378 | 12654675 | transcobalamin II; macrocytic anemia |
| 1379 | 12655133 | CGI-63 protein, similar to |
| 1380 | 12655157 | centrosomal protein 2 |
| 1381 | 12655195 | heat shock 75 protein |
| 1382 | 12656979 | antigen, T-cell receptor |
| 1383 | 12657106 | Unknown |
| 1384 | 12659007 | protein kinase D2 |
| 1385 | 12669909 | long-chain fatty-acid-Coenzyme A ligase 4, isoform 2; long-chain acyl- |
| | | CoA synthetase 4; acyl-activating enzyme |
| 1386 | 12697312 | putative chromatin modulator |
| 1387 | 12697482 | novel zinc finger protein similar to rat RIN ZF) |
| 1388 | 12697776 | polyadenylation specificity factor |
| 1389 | 12697899 | Unknown |
| 1390 | 12697903 | Unknown |
| 1391 | 12697947 | Unknown |
| 1392 | 12697951 | Unknown |
| 1393 | 12697957 | Unknown |
| 1394 | 12697983 | Unknown |
| 1395 | 12697991 | Unknown |
| 1396 | 12697995 | Unknown |
| 1397 | 12698037 | Unknown |
| 1398 | 12698043 | Unknown |
| 1399 | 12698057 | likley ortholog of rat CPG2 protein |
| 1400 | 12698069 | Unknown |
| 1401 | 12698075 | Unknown |
| 1402 | 12700223 | recombination activating protein 1 |
| 1403 | 12707570 | enoyl Coenzyme A hydratase, short chain, 1, mitochondrial |
| 1404 | 12711660 | protein kinase, lysine deficient 1 |
| 1405 | 12711664 | Unknown |
| 1406 | 12711674 | yeast Upf3, variant B, similar to |
| 1407 | 12725813 | poly(ADP-ribosyl)transferase |
| 1408 | 12729652 | cell adhesion molecule with homology to L1CAM (close homologue of L1) |
| 1409 | 12733033 | caldesmon 1 or) NAG22 protein |
| 1410 | 12733091 | replication initiation region protein (60 kD) |
| 1411 | 12734392 | annexin A13 |
| 1412 | 12734816 | PRP4/STK/WD splicing factor |
| 1413 | 12735217 | surfeit 5 |
| 1414 | 12735226 | adenylate kinase 3 alpha |
| 1415 | 12735430 | PKCq-interacting protein PICOT |
| 1416 | 12738042 | klotho |
| 1417 | 12738974 | Unknown |
| 1418 | 12740808 | A kinase anchor protein 10 |
| 1419 | 12741202 | UDP-Gal: betaGlcNAc beta 1,4-galactosyltransferase |
| 1420 | 12741866 | protein expressed in thyroid, similar to |
| 1421 | 12742008 | chondroitin sulfate proteoglycan 3 |
| 1422 | 12742415 | complement component C1q receptor |
| 1423 | 12751117 | PNAS-140 |
| 1424 | 12751119 | PNAS-141 |
| 1425 | 12751452 | PDZ domain-containing protein AIPC |
| 1426 | 12803243 | Unknown |
| 1427 | 12803281 | VDAC-3 |
| 1428 | 12803349 | transcription factor 19, similar to |
| 1429 | 12803387 | antiquitin 1 |
| 1430 | 12803567 | transgelin 2 |
| 1431 | 12803843 | protein kinase, cAMP-dependent, regulatory, type II, alpha, similar to |
| 1432 | 12803855 | metastasis suppressor protein |
| 1433 | 12803915 | glucosidase I, similar to |
| 1434 | 12804041 | nuclear protein E3-3 orf1 |
| 1435 | 12804069 | FK506-binding protein 4 (59 kD), similar to |
| 1436 | 12804185 | colon cancer-associated protein Mic1, similar to |
| 1437 | 12804225 | Unknown |
| 1438 | 12804313 | expressed sequence 2 embryonic lethal, similar to |
| 1439 | 12804319 | carbonyl reductase |
| 1440 | 12804667 | Unknown |
| 1441 | 12804743 | Unknown |
| 1442 | 12804755 | NPD002 protein, similar to |
| 1443 | 12804821 | Unknown |
| 1444 | 12804897 | branched chain aminotransferase 2, mitochondrial, similar to |
| 1445 | 12804901 | isocitrate dehydrogenase 3 gamma |
| 1446 | 12805021 | acyl-Coenzyme A dehydrogenase family, member 8 |
| 1447 | 12805031 | roundabouth |
| 1448 | 12830367 | serine/threonine kinase 33 |
| 1449 | 12862320 | WDC146 |
| 1450 | 12963353 | fenestrated-endothelial linked structure protein |
| 1451 | 13027604 | mitochondrial ribosomal protein S34 |
| 1452 | 13027608 | Unknown |
| 1453 | 13027640 | lysine-ketoglutarate reductase/saccharopine dehydrogenase |
| 1454 | 13095054 | ovarian immunoreactive antigen |
| 1455 | 13096727 | Smac Bound To Xiap-Bir3 Domain |
| 1456 | 13096755 | Ras G12v - Pi 3-Kinase Gamma Complex |
| 1457 | 13097156 | ND 39 k |
| 1458 | 13097243 | Unknown |
| 1459 | 13097693 | Unknown |
| 1460 | 13111705 | Carnitine O-acetyltransferase (Carnitine acetylase) (CAT) |
| 1461 | 13111762 | solute carrier family 19 (folate transporter), member 1, similar to |
| 1462 | 13112023 | coenzyme Q, 7homolog |
| 1463 | 13123976 | ARGININE-TRNA-PROTEIN TRANSFERASE 1 |
| 1464 | 13124237 | F-box only protein 10 |
| 1465 | 13124883 | HsKin17 protein |
| 1466 | 13128992 | Unknown |
| 1467 | 13128998 | Unknown |
| 1468 | 13129014 | Unknown |
| 1469 | 13129080 | Unknown |
| 1470 | 13129092 | Unknown |
| 1471 | 13129144 | Unknown |
| 1472 | 13161081 | testis protein |
| 1473 | 13177634 | surfactant protein B-binding protein |
| 1474 | 13177648 | EGF factor 8 protein |
| 1475 | 13177700 | Unknown |
| 1476 | 13184052 | butyrophilin, subfamily 2, member A3 |
| 1477 | 13194197 | kinesin family member 13B; guanylate kinase associated kinesin |
| 1478 | 13194522 | PMF-1 binding protein |
| 1479 | 13236495 | quinone oxidoreductase; NADPH |
| 1480 | 13236559 | Unknown |
| 1481 | 13242069 | nuclear transcription factor NFX2 |
| 1482 | 13242172 | potassium voltage-gated channel, Shab-related subfamily, member 2 |
| 1483 | 13242739 | myelin P2 protein |
| 1484 | 13249985 | Lowe oculocerebrorenal syndrome protein |
| 1485 | 13259127 | cullin CUL4B |
| 1486 | 13259497 | retinoblastoma-binding protein 1, isoform I |
| 1487 | 13272567 | ND 5 |
| 1488 | 13272568 | ND 6 |
| 1489 | 13272595 | ND 5 NADH dehydrogenase subunit 5 |
| 1490 | 13272697 | ND 1 NADH dehydrogenase subunit 1 |
| 1491 | 13272855 | ATPase 8 |
| 1492 | 13273190 | cox 2 |
| 1493 | 13274124 | Unknown |
| 1494 | 13276227 | chromogranin B(isoform 2) |
| 1495 | 13276598 | Unknown |
| 1496 | 13276617 | Unknown |
| 1497 | 13278690 | Unknown |
| 1498 | 13324710 | interleukin 3 receptor, alpha (low affinity); Interleukin-3 |
| 1499 | 13325066 | cadherin EGF LAG seven-pass G-type receptor 3; EGF-like-domain |
| 1500 | 13325162 | Unknown |
| 1501 | 13325394 | phosphatidylserine synthase 1, similar to |
| 1502 | 13359201 | Unknown |
| 1503 | 13375614 | peroxisomal long-chain acyl-coA thioesterase |
| 1504 | 13375634 | human immunodeficiency virus type I enhancer-binding protein |
| 1505 | 13375744 | Unknown |
| 1506 | 13375809 | Unknown |
| 1507 | 13375817 | Unknown |
| 1508 | 13375838 | Unknown |
| 1509 | 13375872 | Unknown |
| 1510 | 13375932 | Unknown |
| 1511 | 13375940 | Unknown |
| 1512 | 13375942 | Unknown |
| 1513 | 13376007 | Unknown |
| 1514 | 13376011 | engulfment and cell motility 3; ced-12 homolog 3 |
| 1515 | 13376021 | Unknown |
| 1516 | 13376038 | Unknown |
| 1517 | 13376052 | Unknown |
| 1518 | 13376093 | Unknown |
| 1519 | 13376107 | Unknown |
| 1520 | 13376144 | Unknown |
| 1521 | 13376284 | Unknown |
| 1522 | 13376331 | Unknown |
| 1523 | 13376437 | Unknown |
| 1524 | 13376445 | Unknown |
| 1525 | 13376490 | Unknown |
| 1526 | 13376580 | Unknown |
| 1527 | 13376617 | Unknown |
| 1528 | 13376640 | putative N-acetyltransferase |
| 1529 | 13376662 | Unknown |
| 1530 | 13376717 | Unknown |
| 1531 | 13376741 | Unknown |
| 1532 | 13376747 | Unknown |
| 1533 | 13376749 | Unknown |
| 1534 | 13376776 | Unknown |
| 1535 | 13376812 | type 1 protein phosphatase inhibitor |
| 1536 | 13376826 | UL16-binding protein 1 |
| 1537 | 13376854 | UBX domain-containing 1; UBX domain-containing 2 |
| 1538 | 13376991 | voltage-dependent calcium channel beta 2 subunit |
| 1539 | 13386494 | Unknown |
| 1540 | 13399777 | Macrophage Migration Inhibitory Factor (Mif) Complexed With Inhibitor. |
| 1541 | 13431759 | PARAPLEGIN |
| 1542 | 13431763 | Pre-mRNA cleavage complex II protein Pcf11 |
| 1543 | 13435131 | WW domain-containing binding protein 4 |
| 1544 | 13435350 | ferredoxin reductase isoform 1 |
| 1545 | 13436080 | cleft lip and palate associated transmembrane protein 1 |
| 1546 | 13436188 | mitochondrial ribosomal protein S2 |
| 1547 | 13436197 | Unknown |
| 1548 | 13436275 | LON PROTEASE HOMOLOG, MITOCHONDRIAL PRECURSOR |
| 1549 | 13436296 | Unknown |
| 1550 | 13436308 | Unknown |
| 1551 | 13436335 | IF-1 ATPase inhibitor precursor |
| 1552 | 13436395 | Unknown |
| 1553 | 13436413 | glucose phosphate isomerase |
| 1554 | 13445577 | EDAG |
| 1555 | 13449263 | Unknown |
| 1556 | 13449269 | Unknown |
| 1557 | 13469731 | breast cancer antigen NY-BR-1.1 |
| 1558 | 13470094 | apolipoprotein L, 5 |
| 1559 | 13477253 | Unknown |
| 1560 | 13487904 | Unknown |
| 1561 | 13489087 | serine (or cysteine) proteinase inhibitor, clade B (ovalbumin), member 1; |
| | | protease inhibitor 2 (anti-elastase) |
| 1562 | 13489095 | sialoadhesin precursor; sialic acid-binding immunoglobulin-like lectin 1 |
| 1563 | 13491972 | liver nuclear protein |
| 1564 | 13507059 | ubiquitin protein ligase |
| 1565 | 13509322 | suppression of tumorigenicity 5 |
| 1566 | 13514831 | DEAD/H (Asp-Glu-Ala-Asp/His) box polypeptide 10, ATP-dependent RNA |
| | | helicase |
| 1567 | 13516379 | aldehyde oxidase 1 |
| 1568 | 13518228 | methylcrotonoyl-Coenzyme A carboxylase |
| 1569 | 13528660 | ribosomal protein L4, similar to |
| 1570 | 13528960 | ND 18k |
| 1571 | 13529047 | transforming growth factor, alpha |
| 1572 | 13529221 | PTD017 protein |
| 1573 | 13529257 | aldo-keto reductase family 1, member B1 |
| 1574 | 13537192 | SCCA1b |
| 1575 | 13540475 | serum amyloid A2 |
| 1576 | 13540477 | wingless-type MMTV integration site family, member 3 precursor |
| 1577 | 13540574 | Unknown |
| 1578 | 13540576 | Unknown |
| 1579 | 13540590 | C/EBP-induced protein |
| 1580 | 13540606 | suppressor of potassium transport defect 3 g |
| 1581 | 13543342 | Unknown |
| 1582 | 13543446 | Unknown |
| 1583 | 13543618 | ATPase B F0 |
| 1584 | 13543706 | Unknown |
| 1585 | 13543933 | Unknown |
| 1586 | 13544007 | Unknown |
| 1587 | 13544072 | glycerol-3-phosphate dehydrogenase 1 (soluble), similarity to |
| 1588 | 13559241 | Unknown |
| 1589 | 13559363 | mitochondrial ribosomal protein L9 |
| 1590 | 13559404 | mitochondrial ribosomal protein L43 |
| 1591 | 13560110 | Unknown |
| 1592 | 13569848 | cell cycle progression 2 protein |
| 1593 | 13569913 | exonuclease NEF-sp |
| 1594 | 13569930 | toll-like receptor 10 |
| 1595 | 13569948 | Unknown |
| 1596 | 13569962 | small GTP-binding protein |
| 1597 | 13591536 | Unknown |
| 1598 | 13606056 | DNA dependent protein kinase catalytic subunit |
| 1599 | 13620885 | mitochondrial ribosomal protein S6 |
| 1600 | 13623251 | transcription factor EB, similar to |
| 1601 | 13623369 | Unknown |
| 1602 | 13623465 | peroxisomal long-chain acyl-coA thioesterase |
| 1603 | 13623483 | lysosomal-associated membrane protein 1 |
| 1604 | 13623595 | DNA segment on chromosome 191177 expressed sequence |
| 1605 | 13623615 | Unknown |
| 1606 | 13623617 | Unknown |
| 1607 | 13623635 | Unknown |
| 1608 | 13623689 | Unknown |
| 1609 | 13623693 | Unknown |
| 1610 | 13626125 | ADAM-TS-9 precursor (A disintegrin and metalloproteinase with |
| | | thrombospondin motifs 9) (ADAM-TS 9) (ADAM-TS9) |
| 1611 | 13627233 | aldo-keto reductase family 7, member A3 |
| 1612 | 13627252 | oxoglutarate dehydrogenase |
| 1613 | 13627389 | elongation factor-2 kinase |
| 1614 | 13627804 | acyl-Coenzyme A dehydrogenase, short/branched chain precursor |
| 1615 | 13628614 | Na, K-ATPase subunit alpha 2 |
| 1616 | 13628881 | Unknown |
| 1617 | 13629150 | cox 4 |
| 1618 | 13630128 | faciogenital dysplasia protein |
| 1619 | 13630492 | Unknown |
| 1620 | 13630567 | Unknown |
| 1621 | 13630862 | aldehyde dehydrogenase 5 family, member A1 |
| 1622 | 13630871 | Unknown |
| 1623 | 13630873 | protein kinase, cAMP-dependent, regulatory, type II, beta |
| 1624 | 13631242 | reelin |
| 1625 | 13631440 | PEROXIREDOXIN 2 |
| 1626 | 13631521 | mitochondrial ribosomal protein S7 |
| 1627 | 13631678 | UCR 5 |
| 1628 | 13631907 | mitogen-activated protein kinase kinase kinase kinase 1 |
| 1629 | 13632179 | myosin, heavy polypeptide 13, skeletal muscle |
| 1630 | 13632266 | thyroid hormone receptor interactor 2; PPARG binding protein |
| 1631 | 13632616 | carrier ANT2 |
| 1632 | 13632896 | phosphoglucomutase 1 |
| 1633 | 13633168 | plastin 3 precursor |
| 1634 | 13633370 | Notchhomolog 3 |
| 1635 | 13635754 | CTCL tumor antigen se1-1 |
| 1636 | 13635919 | Unknown (now 4507953) |
| 1637 | 13636042 | Unknown |
| 1638 | 13636047 | 3-hydroxyisobutyryl-Coenzyme A hydrolase |
| 1639 | 13636157 | Unknown |
| 1640 | 13636168 | eukaryotic translation elongation factor 1 beta 2 |
| 1641 | 13636504 | interferon-induced protein 75, 52 kD |
| 1642 | 13636598 | Unknown |
| 1643 | 13637083 | Unknown |
| 1644 | 13637529 | Unknown |
| 1645 | 13637537 | ETAA16 protein |
| 1646 | 13637608 | ND 75 K |
| 1647 | 13637631 | VDAC-2 voltage-dependent anion channel 2 (H. sapiens), similar to |
| 1648 | 13637711 | glycine cleavage system protein H (aminomethyl carrier) (H. sapiens), |
| | | similar to |
| 1649 | 13637735 | Unknown |
| 1650 | 13637796 | Unknown |
| 1651 | 13637833 | cox 7a like, COX7RP (cytochrome c oxidase subunit VII-related protein), |
| | | estrogen receptor binding CpG island |
| 1652 | 13637948 | glutathione S-transferase M5 |
| 1653 | 13638591 | Unknown |
| 1654 | 13638758 | Unknown |
| 1655 | 13639105 | Unknown |
| 1656 | 13639114 | succinate dehydrogenase, Ip |
| 1657 | 13639187 | Unknown |
| 1658 | 13639470 | Unknown |
| 1659 | 13639628 | acetyl-Coenzyme A acetyltransferase 1 (acetoacetyl Coenzyme A |
| | | thiolase), mitochondrial |
| 1660 | 13639817 | malic enzyme 3, NADP(+)-dependent, mitochondrial |
| 1661 | 13640712 | phosphoinositide-3-kinase, class 2, alpha polypeptide |
| 1662 | 13640950 | interleukin 11 receptor, alpha |
| 1663 | 13641918 | sirtuin 3 |
| 1664 | 13643253 | kinesin family member 3A |
| 1665 | 13643321 | Unknown |
| 1666 | 13643514 | Unknown |
| 1667 | 13643534 | ribosomal protein L12; 60S ribosomal protein L12 (H. sapiens), similar to |
| 1668 | 13643564 | exostoses 1 |
| 1669 | 13643652 | flavohemoprotein b5 + b5R |
| 1670 | 13643704 | protein tyrosine phosphatase, receptor type |
| 1671 | 13644108 | proteasome 26S subunit, non-ATPase, 1 |
| 1672 | 13644418 | Unknown |
| 1673 | 13644786 | butyrophilin, subfamily 1, member A1 |
| 1674 | 13645381 | HLA-B associated transcript 2 (H. sapiens), similar to |
| 1675 | 13645492 | heat shock 70 kD protein-like 1 |
| 1676 | 13645618 | dihydropyrimidinase related protein-3 |
| 1677 | 13646385 | creatine kinase, sarcomeric mitochondrial |
| 1678 | 13646774 | quinoid dihydropteridine reductase |
| 1679 | 13647276 | L-3-hydroxyacyl-Coenzyme A dehydrogenase, short chain |
| 1680 | 13647558 | carrier ANT1 |
| 1681 | 13647920 | gamma-glutamyltransferase 1 |
| 1682 | 13647960 | tumor necrosis factor, alpha-induced protein 2 |
| 1683 | 13648234 | Unknown |
| 1684 | 13648426 | cox assembly protein isoform 2 |
| 1685 | 13648611 | serine/threonine kinase 2 |
| 1686 | 13648964 | alanyl-tRNA synthetase |
| 1687 | 13649010 | odzhomolog 1 |
| 1688 | 13649058 | Unknown |
| 1689 | 13649119 | SEX gene |
| 1690 | 13649217 | VDAC-1 |
| 1691 | 13649475 | Unknown |
| 1692 | 13649658 | UCR ubiquinol-cytochrome c reductase binding protein |
| 1693 | 13650446 | heat shock 70 kD protein 2 |
| 1694 | 13650574 | glutamate dehydrogenase 2 mitochondrial precursor |
| 1695 | 13650639 | melanoma antigen, family B, 1 |
| 1696 | 13650785 | spectrin, beta, non-erythrocytic 5 |
| 1697 | 13650793 | elongation factor SIII p15 subunit |
| 1698 | 13650874 | putative receptor protein |
| 1699 | 13650942 | Unknown |
| 1700 | 13650992 | Unknown |
| 1701 | 13651038 | leukocyte immunoglobulin-like receptor, subfamily B (with TM and ITIM |
| | | domains), member 4 |
| 1702 | 13651229 | Rho GTPase activating protein 6 isoform 4 |
| 1703 | 13651413 | Fc fragment of IgG binding protein (H. sapiens), similar to |
| 1704 | 13651526 | androgen-induced prostate proliferative shutoff associated protein |
| 1705 | 13651706 | golgin-like protein |
| 1706 | 13651985 | type 1 RNA helicase pNORF1 or nonsense-mediated mRNA decay |
| | | trans-acting factor |
| 1707 | 13652204 | Unknown |
| 1708 | 13652240 | ribosomal protein S7 |
| 1709 | 13652246 | ARF protein |
| 1710 | 13652324 | ras-related small GTPasehypothetical protein X |
| 1711 | 13652801 | Rap1 guanine-nucleotide-exchange factor directly activated by cA |
| 1712 | 13653049 | acyl-Coenzyme A dehydrogenase, C-2 to C-3 short chain precursor |
| 1713 | 13653910 | carboxypeptidase D precursor |
| 1714 | 13654274 | Unknown |
| 1715 | 13654278 | Unknown |
| 1716 | 13654294 | Unknown |
| 1717 | 13654678 | Unknown |
| 1718 | 13654685 | ATP-binding cassette, sub-family C, member 1, isoform 6 |
| 1719 | 13655145 | UCR ubiquinol-cytochrome c reductase, Rieske iron-sulfur polypeptide- |
| | | like 1 |
| 1720 | 13655148 | EH-domain containing 2; EH domain containing 2, similar to |
| 1721 | 13655297 | Unknown |
| 1722 | 13676336 | Unknown |
| 1723 | 13676857 | heat shock 70 kD protein 2; Heat-shock 70 kD protein-2 |
| 1724 | 13699811 | WHSC1L1 protein isoform long; Wolf-Hirschhorn syndrome candidate 1- |
| | | like 1 protein |
| 1725 | 13751974 | Unknown |
| 1726 | 13774961 | autoimmune infertility-related protein |
| 1727 | 13775158 | Unknown |
| 1728 | 13775166 | Unknown |
| 1729 | 13775186 | ring finger protein 17 isoform long |
| 1730 | 13775208 | Unknown |
| 1731 | 13775210 | Unknown |
| 1732 | 13775216 | Unknown |
| 1733 | 13775232 | Unknown |
| 1734 | 13784938 | Unknown |
| 1735 | 13786129 | RAS-RELATED PROTEIN RAB-33B |
| 1736 | 13786847 | L-Lactate Dehydrogenase H Chain, Ternary Complex With Nadh And |
| | | Oxamate |
| 1737 | 13787197 | DEAD/Hbox polypeptide 11 |
| 1738 | 13787215 | sirtuin 5, isoform 2 |
| 1739 | 13787217 | FAT tumor suppressor 2 precursor; multiple epidermal growth factor-like |
| | | domains 1; cadherin family member 8 |
| 1740 | 13794267 | RAB7, member RAS oncogene family; Ras-associated protein RAB |
| 1741 | 13872241 | ligand of numb-protein X |
| 1742 | 13874437 | cerebral protein-11 |
| 1743 | 13876386 | epiplakin 1 |
| 1744 | 13899231 | mitochondrial ribosomal protein L9 |
| 1745 | 13899275 | Unknown |
| 1746 | 13929460 | PTH-responsive osteosarcoma B1 protein |
| 1747 | 13929467 | chemokine binding protein 2 |
| 1748 | 13937401 | Unknown |
| 1749 | 13937769 | RIKEN cDNA 1200013F24 gene, similar to |
| 1750 | 13937888 | heterogeneous nuclear ribonucleoprotein C |
| 1751 | 13938170 | Unknown |
| 1752 | 13938215 | taxol resistant associated protein |
| 1753 | 13938297 | heat shock cognate 71-kd protein, similar to |
| 1754 | 13938442 | neuronal protein, mitochondrial Complex I subunit |
| 1755 | 13938539 | cyclin D binding Myb-like transcription factor 1 |
| 1756 | 13938571 | Unknown |
| 1757 | 13938593 | Unknown |
| 1758 | 13938619 | creatine kinase, muscle |
| 1759 | 13994164 | Charcot-Marie-Tooth duplicated region transcript 1 |
| 1760 | 13994188 | AKAP-associated sperm protein |
| 1761 | 13994259 | mitochondrial ribosomal protein S5 |
| 1762 | 13994280 | complement-c1q tumor necrosis factor-related protein 7 + F792 |
| 1763 | 13994325 | putative b,b-carotene-9′, 10′-dioxygenase |
| 1764 | 14017783 | Unknown |
| 1765 | 14017783 | Unknown |
| 1766 | 14017807 | Unknown |
| 1767 | 14017833 | Unknown |
| 1768 | 14017865 | Unknown |
| 1769 | 14017899 | Unknown |
| 1770 | 14017903 | Unknown |
| 1771 | 14017903 | Unknown |
| 1772 | 14017923 | Unknown |
| 1773 | 14017941 | Unknown |
| 1774 | 14017943 | Unknown |
| 1775 | 14017949 | Unknown |
| 1776 | 14017957 | Unknown |
| 1777 | 14017971 | Unknown |
| 1778 | 14028389 | mitochondrial ribosomal protein L41 |
| 1779 | 14028403 | mitochondrial ribosomal protein S28 |
| 1780 | 14028405 | mitochondrial ribosomal protein S29 |
| 1781 | 14028875 | UDP-glucuronic acid/UDP-N-acetylgalactosamine dual transporter; |
| | | KIAA0260 protein; UDP-glucuronic acid/UDP-N-acetylgalactosamine dual |
| | | transporter |
| 1782 | 14028877 | mitochondrial ribosomal protein S25; mitochondrial 28S ribosomal protein |
| | | S25 |
| 1783 | 14041699 | ESTRADIOL 17 BETA-DEHYDROGENASE 8 |
| 1784 | 14041874 | MAPKK like protein kinase/PDZ-binding kinase |
| 1785 | 14041889 | Unknown |
| 1786 | 14041976 | Unknown |
| 1787 | 14041978 | CDA02 protein |
| 1788 | 14041989 | Unknown |
| 1789 | 14042018 | Unknown |
| 1790 | 14042066 | Unknown |
| 1791 | 14042110 | Unknown |
| 1792 | 14042216 | Unknown |
| 1793 | 14042323 | Unknown |
| 1794 | 14042336 | Unknown |
| 1795 | 14042441 | Unknown |
| 1796 | 14042814 | Unknown |
| 1797 | 14042822 | Unknown |
| 1798 | 14042850 | Unknown |
| 1799 | 14042923 | chromosome 9 open reading frame 5 |
| 1800 | 14043187 | aldehyde dehydrogenase 4 A1 |
| 1801 | 14043217 | plectin 1, intermediate filament bindi |
| 1802 | 14043281 | leucine-rich neuronal protein |
| 1803 | 14043412 | Unknown |
| 1804 | 14043451 | succinyl-CoA synthetase beta subunit GTP-specific |
| 1805 | 14043654 | phosphofructokinase, muscle, similar to |
| 1806 | 14043666 | Unknown |
| 1807 | 14043738 | Unknown |
| 1808 | 14124942 | ribophorin I, similar to |
| 1809 | 14124976 | kinesin family member C3 |
| 1810 | 14133213 | Unknown |
| 1811 | 14133215 | Unknown |
| 1812 | 14133217 | Unknown |
| 1813 | 14133235 | Unknown |
| 1814 | 14141157 | heterogeneous nuclear ribonucleoprotein H3, isoform a |
| 1815 | 14149607 | chloride channel 7; CIC-7 |
| 1816 | 14149625 | ND 20 k |
| 1817 | 14149649 | siah binding protein 1; FBP interacting repressor; pyrimidine tract binding |
| | | splicing factor; Ro ribonucleoprotein-binding protein 1 |
| 1818 | 14149677 | lectomedin-3 |
| 1819 | 14149686 | Unknown |
| 1820 | 14149690 | Unknown |
| 1821 | 14149769 | GAJ protein |
| 1822 | 14149789 | Unknown |
| 1823 | 14149904 | tumor endothelial marker 8, isoform 1 precursor; anthrax toxin receptor |
| 1824 | 14149971 | Unknown |
| 1825 | 14150001 | Unknown |
| 1826 | 14150017 | Unknown |
| 1827 | 14150039 | Unknown |
| 1828 | 14150062 | Unknown |
| 1829 | 14150072 | Unknown |
| 1830 | 14150072 | Unknown |
| 1831 | 14150080 | Unknown |
| 1832 | 14150116 | Unknown |
| 1833 | 14150128 | phosphodiesterase 5A |
| 1834 | 14150134 | Unknown |
| 1835 | 14150155 | Unknown |
| 1836 | 14165260 | Unknown |
| 1837 | 14165270 | mitochondrial ribosomal protein L13 |
| 1838 | 14192943 | MEGF10 protein |
| 1839 | 14194461 | A kinase anchor protein 9 |
| 1840 | 14196457 | protocadherin gamma subfamily A, 12, isoform 2 precursor; cadherin 21; |
| | | fibroblast cadherin FIB3 |
| 1841 | 14196465 | protocadherin gamma subfamily A, 3, isoform 2 precursor |
| 1842 | 14198176 | ND 51 k |
| 1843 | 14198272 | Bcl-XL-binding protein v68, similar to |
| 1844 | 14198303 | Unknown |
| 1845 | 14211536 | neurexin 2; neurexin II |
| 1846 | 14211570 | conserved ERA-like GTPase |
| 1847 | 14211720 | desmuslin |
| 1848 | 14211857 | Unknown |
| 1849 | 14211903 | ubiquitin specific protease |
| 1850 | 14211907 | zinc finger protein 347; zinc finger 1111 |
| 1851 | 14211923 | PKCI-1-related HIT protein |
| 1852 | 14211939 | methylmalonyl-CoA epimerase |
| 1853 | 14248761 | cAMP-specific cyclic nucleotide phosphodiesterase |
| 1854 | 14249144 | RAB11B, member RAS oncogene family |
| 1855 | 14249338 | Unknown |
| 1856 | 14249342 | internexin neuronal intermediate filament protein, alpha; neurofilament 5 |
| | | (66 kD); neurofilament-66, tax-binding protein |
| 1857 | 14249376 | Unknown |
| 1858 | 14249428 | Unknown |
| 1859 | 14249446 | Unknown |
| 1860 | 14249454 | Unknown |
| 1861 | 14249474 | Unknown |
| 1862 | 14249506 | Unknown |
| 1863 | 14249588 | lactamase, beta |
| 1864 | 14249596 | Unknown |
| 1865 | 14249620 | Unknown |
| 1866 | 14249967 | staufenhomolog 2 |
| 1867 | 14250063 | peroxiredoxin 3 |
| 1868 | 14250110 | Unknown |
| 1869 | 14250319 | Unknown |
| 1870 | 14250458 | stromal cell derived factor 5, similar to |
| 1871 | 14250628 | Unknown |
| 1872 | 14250744 | Unknown |
| 1873 | 14251209 | chloride intracellular channel 1 |
| 1874 | 14269578 | metallothionein IV |
| 1875 | 14277739 | Erythrocyte Band-3 Protein, Crystal Structure Of The Cytoplasmic Domain |
| | | Of Human |
| 1876 | 14280050 | Vps39/Vam6-like protein |
| 1877 | 14285174 | elongation factor G |
| 1878 | 14286186 | ZINC FINGER PROTEIN 185(P1-A) g |
| 1879 | 14286294 | Unknown |
| 1880 | 14289323 | LIP isoform of BLIP |
| 1881 | 14318622 | Unknown |
| 1882 | 14329511 | bA430M15.1 (novel protein (ortholog of rat four repeat ion channel)) |
| 1883 | 14329531 | Unknown |
| 1884 | 14336727 | Unknown |
| 1885 | 14336768 | Unknown |
| 1886 | 14336775 | ND PDSW |
| 1887 | 14349362 | major histocompatibility complex, class I, F |
| 1888 | 14424013 | WNT-5B protein precursor |
| 1889 | 14424776 | Unknown |
| 1890 | 14485049 | T-cell receptor V delta 1 |
| 1891 | 14488680 | Phosphoglucose IsomeraseNEUROLEUKINAUTOCRINE MOTILITY |
| | | FACTORMATURATION Factor |
| 1892 | 14530763 | citrate lyase, similar to |
| 1893 | 14549163 | Matrilin-2 precursor |
| 1894 | 14571713 | tonicity-responsive enhancer binding protein |
| 1895 | 14575679 | hemicentin |
| 1896 | 14602477 | DNA-BINDING PROTEIN A |
| 1897 | 14602507 | Unknown |
| 1898 | 14602841 | cysteine string protein 1 |
| 1899 | 14602856 | Unknown |
| 1900 | 14602907 | Unknown |
| 1901 | 14602977 | Unknown |
| 1902 | 14603084 | putative DNA binding protein |
| 1903 | 14603309 | heat shock 60 kD MITOCHONDRIAL |
| 1904 | 14603403 | stomatin-like 2 |
| 1905 | 14670360 | zinc finger protein 278, long C isoform; POZ-AT hook-zinc finger protein |
| 1906 | 14714447 | sorting nexin 7 |
| 1907 | 14714514 | DIHYDROLIPOAMIDE DEHYDROGENASE-BINDING PROTEIN OF |
| | | PYRUVATE DEHYDROGENASE COMPLEX |
| 1908 | 14714528 | Unknown |
| 1909 | 14715007 | Unknown |
| 1910 | 14719392 | cofilin 2 |
| 1911 | 14720172 | Unknown |
| 1912 | 14720558 | succinate dehydrogenase, flavoprotein subunit |
| 1913 | 14721241 | low density lipoprotein-related protein-associated protein 1 |
| 1914 | 14721350 | testicular protein kinase 2 |
| 1915 | 14721365 | hypothetical protein, estradiol-induced |
| 1916 | 14721507 | serine/threonine kinase 18 |
| 1917 | 14721966 | Unknown |
| 1918 | 14722003 | cadherin 12, type 2 |
| 1919 | 14722193 | 3-hydroxybutyrate dehydrogenase |
| 1920 | 14722283 | Unknown |
| 1921 | 14722554 | Unknown |
| 1922 | 14722589 | mitochondrial ribosomal protein L22 |
| 1923 | 14722898 | mitochondrial ribosomal protein S27 |
| 1924 | 14723145 | acid phosphatase 1 isoform b |
| 1925 | 14723407 | Unknown |
| 1926 | 14723451 | mitochondrial ribosomal protein L20 |
| 1927 | 14723531 | p25 |
| 1928 | 14724042 | ASB-3 protein |
| 1929 | 14724206 | Unknown |
| 1930 | 14724379 | Unknown |
| 1931 | 14724557 | phosphatidylinositol glycan, class K |
| 1932 | 14724575 | Unknown |
| 1933 | 14724751 | phosphorylase, glycogen; brain |
| 1934 | 14724805 | Unknown |
| 1935 | 14725181 | lymphocyte antigen 75 |
| 1936 | 14725399 | TNF-induced protein |
| 1937 | 14725420 | syntaxin 12 |
| 1938 | 14725545 | RNA-binding protein regulatory subunit |
| 1939 | 14725791 | Unknown |
| 1940 | 14725848 | acyl-Coenzyme A dehydrogenase, C-4 to C-12 straight chain |
| 1941 | 14726372 | Unknown |
| 1942 | 14726632 | Unknown |
| 1943 | 14726693 | Unknown |
| 1944 | 14726725 | Unknown |
| 1945 | 14726866 | Unknown |
| 1946 | 14727174 | leucine-rich PPR-motif containing |
| 1947 | 14727486 | succinate dehydrogenase, subunit D |
| 1948 | 14727827 | Unknown |
| 1949 | 14728081 | excision repair cross-complementing rodent repair deficiency |
| 1950 | 14728229 | phosphoinositide-3-kinase, regulatory subunit 4, p150 |
| 1951 | 14728316 | natural killer cell receptor 2B4 |
| 1952 | 14728439 | Unknown |
| 1953 | 14728817 | Unknown |
| 1954 | 14728839 | Unknown |
| 1955 | 14728858 | sterol carrier protein 2 |
| 1956 | 14728945 | DMRT-like family B with proline-rich C-terminal, 1 |
| 1957 | 14729172 | elastin microfibril interface located protein |
| 1958 | 14729475 | BCL9 |
| 1959 | 14729487 | mast cell carboxypeptidase A3 precursor |
| 1960 | 14729783 | dihydrolipoamide branched chain transacylase |
| 1961 | 14730158 | TATA element modulatory factor 1 |
| 1962 | 14730499 | Unknown |
| 1963 | 14730569 | adenylate cyclase 3 |
| 1964 | 14730600 | Unknown |
| 1965 | 14730775 | hydroxyacyl-Coenzyme A dehydrogenase/3-ketoacyl-Coenzyme A |
| | | thiolase/enoyl-Coenzyme A hydratase alpha |
| 1966 | 14730782 | kinesin heavy chain member 2 |
| 1967 | 14732014 | Unknown |
| 1968 | 14732481 | calcium channel, voltage-dependent, alpha 1E subunit |
| 1969 | 14732525 | selective LIM binding factor, rat homolog |
| 1970 | 14732721 | adenomatosis polyposis coli |
| 1971 | 14732789 | mitofilin |
| 1972 | 14732886 | thyroid hormone receptor-associated protein, 150 kDa subunit |
| 1973 | 14733183 | adaptor-related protein complex 2, mu 1 subunit |
| 1974 | 14733451 | enkephalinase |
| 1975 | 14733480 | Unknown |
| 1976 | 14733532 | myofibrillogenesis regulator MR-1 |
| 1977 | 14733712 | chondroitin sulfate proteoglycan 2 |
| 1978 | 14733904 | serine/threonine kinase 16 |
| 1979 | 14734022 | Unknown |
| 1980 | 14734151 | lymphoid enhancer binding factor-1 |
| 1981 | 14734205 | Unknown |
| 1982 | 14734243 | Unknown |
| 1983 | 14734441 | Unknown |
| 1984 | 14734746 | DEAD/Hbox polypeptide 1 |
| 1985 | 14734864 | SWI/SNF related, matrix associated, actin dependent regulator of |
| | | chromatin, subfamily a-like 1 |
| 1986 | 14735060 | mitochondrial isoleucine tRNA synthetase |
| 1987 | 14735128 | Ste-20 related kinase |
| 1988 | 14735161 | BCL6 |
| 1989 | 14735336 | Unknown |
| 1990 | 14735426 | nuclear factor, interleukin 3 regulated |
| 1991 | 14735687 | Unknown |
| 1992 | 14735741 | Unknown |
| 1993 | 14735899 | cytochrome b5 reductase 1 |
| 1994 | 14736223 | UCR 1 |
| 1995 | 14736227 | Rho-associated, coiled-coil containing protein kinase 2 |
| 1996 | 14736267 | protein disulfide isomerase-related protein |
| 1997 | 14736397 | Unknown |
| 1998 | 14736560 | Unknown |
| 1999 | 14736612 | Unknown |
| 2000 | 14736678 | lactotransferrin |
| 2001 | 14736760 | voltage-dependent anion channel 2 |
| 2002 | 14736866 | DnaJhomolog, subfamily B, member 12 |
| 2003 | 14737445 | sema domain, immunoglobulin domain (lg), short basic domain, |
| 2004 | 14737746 | myeloid differentiation primary response gene |
| 2005 | 14737907 | Unknown |
| 2006 | 14738004 | Unknown |
| 2007 | 14738099 | Apobec-1 complementation factor; APOBEC-1 stimulating protein |
| 2008 | 14738103 | annexin IV |
| 2009 | 14738306 | putative, similar to |
| 2010 | 14738689 | Unknown |
| 2011 | 14738950 | Unknown |
| 2012 | 14739002 | Unknown |
| 2013 | 14739106 | Unknown |
| 2014 | 14739392 | Unknown |
| 2015 | 14739472 | potassium voltage-gated channel, shaker-related subfamily |
| 2016 | 14739880 | Unknown |
| 2017 | 14740316 | HEAT SHOCK 27 KDA PROTEIN (HSP 27) (STRESS-RESPONSIVE |
| | | PROTEIN 27) (SRP27) (ESTROGEN-REGULATED 24 KDA PROTEIN) |
| | | (28 KDA HEAT SHOCK PROTEIN), similar to |
| 2018 | 14740371 | A kinase anchor protein 2 |
| 2019 | 14740403 | thioredoxin |
| 2020 | 14740476 | TAF2 RNA polymerase II, TATA box binding protein (TBP)-associated |
| | | factor, 150 kD |
| 2021 | 14740547 | FUMARATE HYDRATASE, MITOCHONDRIAL PRECURSOR |
| | | (FUMARASE) |
| 2022 | 14740792 | v-ral simian leukemia viral oncogene homolog A (ras related) |
| 2023 | 14740886 | Unknown |
| 2024 | 14741177 | Unknown |
| 2025 | 14741234 | Unknown |
| 2026 | 14741376 | Fas-activated serine/threonine kinase, isoform 2 |
| 2027 | 14741510 | Unknown |
| 2028 | 14741555 | Unknown |
| 2029 | 14741636 | Unknown |
| 2030 | 14741782 | uncharacterized hematopoietic stem/progenitor cells protein MDSO |
| 2031 | 14742266 | RNA helicase |
| 2032 | 14742273 | Unknown |
| 2033 | 14742317 | Unknown |
| 2034 | 14742600 | vimentin |
| 2035 | 14742688 | diphthamide biosynthesis-like protein 2 |
| 2036 | 14742977 | inter-alphainhibitor, H2 polypeptide |
| 2037 | 14743031 | Unknown |
| 2038 | 14743873 | TAR (HIV) RNA binding protein 1 |
| 2039 | 14744078 | gamma filamin |
| 2040 | 14744132 | heat shock 70 kD protein 5 (glucose-regulated protein, 78 kD) |
| 2041 | 14744234 | nuclear receptor subfamily 6, group A, member 1, isoform 1 |
| 2042 | 14744290 | Hermansky-Pudlak syndrome protein |
| 2043 | 14744642 | Unknown |
| 2044 | 14744702 | rat myomegalin, similar to |
| 2045 | 14745217 | lipocalin 2 (oncogene 24p3) |
| 2046 | 14745424 | spectrin, alpha, non-erythrocytic 1 (alpha-fodrin) |
| 2047 | 14745489 | wingless-type MMTV integration site family, member 3A |
| 2048 | 14745808 | guanine nucleotide binding proteinalpha 12 |
| 2049 | 14745853 | Z-band alternatively spliced PDZ-motif |
| 2050 | 14745861 | Z-band alternatively spliced PDZ-motif |
| 2051 | 14745865 | Unknown |
| 2052 | 14746475 | Unknown |
| 2053 | 14746487 | ACYL-COA DEHYDROGENASE, VERY-LONG-CHAIN SPECIFIC + F36, |
| | | similar to |
| 2054 | 14746491 | Unknown |
| 2055 | 14746535 | RAB7, member RAS oncogene family |
| 2056 | 14746585 | yeast adenylate cyclase, similar to |
| 2057 | 14747216 | carrier aralar |
| 2058 | 14747249 | CGI-135 protein |
| 2059 | 14747260 | serologically defined colon cancer antigen 1 |
| 2060 | 14747375 | lysophospholipase I |
| 2061 | 14747970 | Unknown |
| 2062 | 14748292 | Unknown |
| 2063 | 14748400 | Unknown |
| 2064 | 14748439 | Unknown |
| 2065 | 14748831 | Unknown |
| 2066 | 14748858 | transformation/transcription domain-associated protein |
| 2067 | 14749079 | vacuolar protein sorting protein 18 |
| 2068 | 14749154 | Unknown |
| 2069 | 14749213 | serine-threonine kinase/MAD3-like protein kinase |
| 2070 | 14749294 | GCN2 elF2alpha kinase |
| 2071 | 14749361 | Unknown |
| 2072 | 14749419 | Unknown |
| 2073 | 14749523 | Unknown |
| 2074 | 14749588 | Unknown |
| 2075 | 14749765 | A kinase anchor protein 6 |
| 2076 | 14749776 | Unknown |
| 2077 | 14750136 | Unknown |
| 2078 | 14750148 | Unknown |
| 2079 | 14750186 | LAMIN A/C (70 KDA LAMIN) |
| 2080 | 14750222 | Unknown |
| 2081 | 14750259 | Rho/Rac guanine nucleotide exchange factor 2 |
| 2082 | 14750405 | pyruvate kinase, muscle (H. sapiens), similar to |
| 2083 | 14751203 | Unknown |
| 2084 | 14751493 | N-acylsphingosine amidohydrolase |
| 2085 | 14751551 | Unknown |
| 2086 | 14751705 | Unknown |
| 2087 | 14751808 | purine nucleoside phosphorylase |
| 2088 | 14751866 | IGF-II mRNA-binding protein 3 |
| 2089 | 14752024 | carrier aralar2 |
| 2090 | 14752229 | dihydrolipoamide dehydrogenase |
| 2091 | 14752236 | Unknown |
| 2092 | 14752239 | laminin, beta 1 precursor |
| 2093 | 14752249 | spectrin, beta, erythrocytic (includes spherocytosis, clinical type I) |
| 2094 | 14752728 | guanine nucleotide exchange factor Lbc or A-kinase anchoring protein |
| 2095 | 14753117 | Unknown |
| 2096 | 14753239 | kinectin 1 |
| 2097 | 14753384 | A kinase (PRKA) anchor protein (gravin) 12 |
| 2098 | 14753693 | adaptor-related protein complex 4, sigma 1 subunit, similar to |
| 2099 | 14753915 | Ras protein-specific guanine nucleotide-releasing factor 1 |
| 2100 | 14754222 | farnesol receptor HRR-1 |
| 2101 | 14754627 | Unknown |
| 2102 | 14754848 | Unknown |
| 2103 | 14754867 | Unknown |
| 2104 | 14755192 | Unknown |
| 2105 | 14755316 | zinc finger protein 91 |
| 2106 | 14755336 | tumor rejection antigen 1 |
| 2107 | 14755347 | Unknown |
| 2108 | 14755357 | mitochondrial ribosomal protein L18 |
| 2109 | 14755436 | superoxide dismutase 2, mitochondrial |
| 2110 | 14755456 | zinc finger protein 256 |
| 2111 | 14755952 | lysophospholipase I, similar to |
| 2112 | 14756295 | Na, K-ATPase subunit alpha 3 |
| 2113 | 14756299 | pot. ORF (1013 AA), similar to |
| 2114 | 14756626 | DNA (cytosine-5)-methyltransferase |
| 2115 | 14756630 | mitochondrial ribosomal protein L4 |
| 2116 | 14756895 | dUTP pyrophosphatase |
| 2117 | 14756939 | Unknown |
| 2118 | 14756944 | Unknown |
| 2119 | 14757147 | Unknown |
| 2120 | 14757210 | FSH primary responsehomolog 1 |
| 2121 | 14757677 | phosphoglycerate kinase 1 |
| 2122 | 14757711 | Unknown |
| 2123 | 14758001 | ND 24K NADH dehydrogenase (ubiquinone) flavoprotein 2 (24 kD) (H. |
| | | sapiens), similar to |
| 2124 | 14758520 | ATPase, Cu++ transporting, beta polypeptide (Wilson disease) |
| 2125 | 14759302 | golgi autoantigen, golgin subfamily a, 3 |
| 2126 | 14759459 | hook2 protein |
| 2127 | 14759609 | Unknown |
| 2128 | 14759903 | transcription factor |
| 2129 | 14759981 | Unknown |
| 2130 | 14760649 | inositol 1,4,5-triphosphate receptor, type 2 |
| 2131 | 14761208 | glyceraldehyde 3-phosphate dehydrogenase like |
| 2132 | 14761398 | tubulin beta 5, similar to |
| 2133 | 14761496 | programmed cell death 8 (apoptosis-inducing factor) |
| 2134 | 14761689 | calcium channel, voltage-dependent, beta 3 subunit |
| 2135 | 14762250 | protein tyrosine phosphatase, receptor type, B |
| 2136 | 14762650 | Unknown |
| 2137 | 14762696 | granzyme M precursor |
| 2138 | 14763105 | Unknown |
| 2139 | 14763304 | src homology 2 domain-containing transforming protein D, similar to |
| 2140 | 14763427 | death-associated protein kinase 3, ZIP-kinase |
| 2141 | 14763491 | NY-REN-58 antigen |
| 2142 | 14763709 | Unknown |
| 2143 | 14763948 | FERM, RhoGEF, and pleckstrin domain protein 1; chondrocyte-derived |
| | | ezrin-like protein, similar to |
| 2144 | 14764159 | acetyl-Coenzyme A acyltransferase 2 (mitochondrial 3-oxoacyl- |
| | | CoenzymeA thiolase) |
| 2145 | 14764202 | hydroxyacyl-Coenzyme A dehydrogenase, type II |
| 2146 | 14764412 | D-amino-acid oxidase |
| 2147 | 14764458 | male-specific lethal-3 (Drosophila)-like 1 |
| 2148 | 14764705 | Unknown |
| 2149 | 14764874 | Unknown |
| 2150 | 14764936 | G protein-coupled receptor 19 |
| 2151 | 14765579 | Unknown |
| 2152 | 14765581 | peroxiredoxin 5 |
| 2153 | 14765684 | kinesin family member 4 |
| 2154 | 14766197 | Unknown |
| 2155 | 14766265 | Unknown |
| 2156 | 14766346 | glutathione S-transferase P1-1 |
| 2157 | 14766373 | regulatory factor X, 4 |
| 2158 | 14766393 | transmembrane protein (63 kD), endoplasmic reticulum/Golgi |
| 2159 | 14766635 | prohibitin, B-cell associated protein |
| 2160 | 14766937 | DRIM protein or Key-1A6 protein |
| 2161 | 14767036 | Unknown |
| 2162 | 14767224 | protein kinase C and casein kinase substrate |
| 2163 | 14767305 | protein C, cardiac |
| 2164 | 14767738 | CALCIUM ATPASE 2(SERCA2) |
| 2165 | 14767795 | Unknown |
| 2166 | 14768227 | purinergic receptor P2X, ligand-gated ion channel, 7 |
| 2167 | 14768743 | thioredoxin peroxidase |
| 2168 | 14769051 | ND B14.5a |
| 2169 | 14769064 | Unknown |
| 2170 | 14769085 | Unknown |
| 2171 | 14769089 | Unknown |
| 2172 | 14769268 | GalNAc alpha-2, 6-sialyltransferase I, long form |
| 2173 | 14769776 | peripheral benzodiazepine receptor-associated protein 1 |
| 2174 | 14770042 | Unknown |
| 2175 | 14770070 | Unknown |
| 2176 | 14770170 | Unknown |
| 2177 | 14770383 | Unknown |
| 2178 | 14770569 | Unknown |
| 2179 | 14770608 | small fragment nuclease |
| 2180 | 14770670 | Unknown |
| 2181 | 14770915 | Unknown |
| 2182 | 14770940 | angiotensin I converting enzyme |
| 2183 | 14770968 | Unknown |
| 2184 | 14771355 | beta-2-glycoprotein I precursor |
| 2185 | 14771369 | brain-immunoglobulin-like molecule with tyrosine-based activation motifs |
| 2186 | 14771396 | isocitrate dehydrogenase 3 beta (NAD+) |
| 2187 | 14771416 | murine retrovirus integration site 1 homolog |
| 2188 | 14771689 | myosin, heavy polypeptide 1, skeletal muscle, adult |
| 2189 | 14772046 | Unknown |
| 2190 | 14772333 | phosphorylase, glycogen; brain (H. sapiens), similar to |
| 2191 | 14772527 | Unknown |
| 2192 | 14772555 | Unknown |
| 2193 | 14772672 | calpain 5 |
| 2194 | 14772954 | copine I |
| 2195 | 14773504 | tyrosine kinase, non-receptor, 1 |
| 2196 | 14773592 | AHNAK nucleoprotein (desmoyokin) |
| 2197 | 14773948 | Unknown |
| 2198 | 14774045 | Unknown |
| 2199 | 14774139 | ATPase g |
| 2200 | 14774236 | Unknown |
| 2201 | 14774282 | apolipoprotein A-I precursor |
| 2202 | 14774359 | ionotropic ATP receptor P2X5b |
| 2203 | 14774503 | phospholipase D2 |
| 2204 | 14774525 | carrier oxoglutarate |
| 2205 | 14774778 | Unknown |
| 2206 | 14774780 | karyopherin (importin) beta 1 |
| 2207 | 14774844 | succinate dehydrogenase, subunit C |
| 2208 | 14775218 | Unknown |
| 2209 | 14775320 | Unknown |
| 2210 | 14775363 | baculoviral IAP repeat-containing protein 5 |
| 2211 | 14775444 | carbohydrate (N-acetylglucosamine 6-O) sulfotransferase 5, similar to |
| 2212 | 14775476 | endocytic receptor (macrophage mannose receptor family) |
| 2213 | 14775546 | malonyl-CoA decarboxylase |
| 2214 | 14775827 | ubiquinol-cytochrome c reductase core protein II |
| 2215 | 14775827 | UCR 2 |
| 2216 | 14776296 | Unknown |
| 2217 | 14776472 | nuclear receptor co-repressor 1 |
| 2218 | 14776681 | Unknown |
| 2219 | 14776736 | Unknown |
| 2220 | 14776778 | ATP-binding cassette, sub-family A member 3 |
| 2221 | 14776800 | cat eye syndrome chromosome region, candidate 5, isoform 1 |
| 2222 | 14776960 | Unknown |
| 2223 | 14776980 | carrier citrate transporter |
| 2224 | 14777215 | protein disulfide isomerase, pancreatic; protein disulfide isomerase, |
| | | similar to |
| 2225 | 14777313 | ND 13 k-B |
| 2226 | 14777483 | general transcription factor IIIC, polypeptide 1 (alpha subunit, 220 kD) |
| 2227 | 14777522 | Unknown |
| 2228 | 14777630 | AT-binding transcription factor 1 |
| 2229 | 14777716 | Unknown |
| 2230 | 14777813 | Unknown |
| 2231 | 14777901 | Unknown |
| 2232 | 14778035 | Unknown |
| 2233 | 14778104 | adaptor-related protein complex 1, beta 1 subunit |
| 2234 | 14778235 | Unknown |
| 2235 | 14778381 | eIF4E-transporter |
| 2236 | 14778431 | ret finger protein-like 2 |
| 2237 | 14778654 | THIOSULFATE SULFURTRANSFERASE (RHODANESE) |
| 2238 | 14779326 | Unknown |
| 2239 | 14779686 | Unknown |
| 2240 | 14779867 | N-acetylglucosamine-1-phosphodiester alpha-N-acetylglucosaminida |
| 2241 | 14779881 | periplakin |
| 2242 | 14779964 | Unknown |
| 2243 | 14780055 | protease, serine, 7 |
| 2244 | 14780117 | Unknown |
| 2245 | 14780193 | synaptojanin 1 |
| 2246 | 14780272 | intersectin 1 (SH3 domain protein) |
| 2247 | 14780668 | ES1 protein /KNP-I protein ?? (ThiJ/Pfpl family motif) |
| 2248 | 14780705 | phosphofructokinase, liver |
| 2249 | 14780857 | Unknown |
| 2250 | 14781094 | huntingtin |
| 2251 | 14781125 | quinoid dihydropteridine reductase (H. sapiens), similar to |
| 2252 | 14781245 | fatty-acid-Coenzyme A ligase, long-chain 6 |
| 2253 | 14781533 | Unknown |
| 2254 | 14781826 | receptor (TNFRSF)-interacting serine-threonine kinase 1 |
| 2255 | 14781890 | Unknown |
| 2256 | 14781979 | Unknown |
| 2257 | 14781989 | putative transcription factor/GTF2I repeat domain-containing 1, isoform 2 |
| 2258 | 14782063 | malate dehydrogenase 2, NAD (mitochondrial) |
| 2259 | 14782332 | HLA-B associated transcript-3, similar to |
| 2260 | 14782751 | Unknown |
| 2261 | 14782921 | protein kinase C and casein kinase substrate in neurons 1 |
| 2262 | 14782973 | tubby like protein 1 |
| 2263 | 14783011 | p38 mitogen-activated protein kinase |
| 2264 | 14783112 | Unknown |
| 2265 | 14783333 | supervillin, isoform 1 |
| 2266 | 14783455 | Unknown |
| 2267 | 14783504 | Unknown |
| 2268 | 14783675 | small GTP binding protein RAB6 isoform |
| 2269 | 14783738 | inositol polyphosphate phosphatase-like 1 |
| 2270 | 14784011 | Unknown |
| 2271 | 14784064 | mitogen-activated protein kinase kinase kinase 11 |
| 2272 | 14784122 | atrophin-1 |
| 2273 | 14784162 | Ubiquitin isopeptidase T |
| 2274 | 14784612 | Unknown |
| 2275 | 14784913 | EH-domain containing 4 |
| 2276 | 14785008 | Unknown |
| 2277 | 14785181 | microfibrillar-associated protein 1 |
| 2278 | 14785356 | Unknown |
| 2279 | 14785405 | polo (Drosophia)-like kinase |
| 2280 | 14785865 | Unknown |
| 2281 | 14785919 | copper containing amine oxidase 3 precursor; amine oxidase (copper- |
| | | containing); copper amine oxidase precursor; vascular adhesion protein 1; |
| | | vascular adhesion protein 1, similar to |
| 2282 | 14786231 | Unknown |
| 2283 | 14786366 | PAR-6 beta |
| 2284 | 14786394 | cytochrome P450, subfamily XXIV precursor |
| 2285 | 14786884 | Unknown |
| 2286 | 14787181 | CUB and sushi multiple domains protein 1 short form |
| 2287 | 14790190 | SMART/HDAC1 associated repressor protein |
| 2288 | 15012003 | Unknown |
| 2289 | 15012048 | HERV-H LTR-associating 3, similar to |
| 2290 | 15020655 | ATP/GTP-binding protein |
| 2291 | 15026974 | obscurin |
| 2292 | 15029619 | fracture callus 1 homolog |
| 2293 | 15029922 | Unknown |
| 2294 | 15030240 | ATPase alpha, H+ transporting, mitochondrial F1 complex, alpha subunit, |
| | | isoform 1, cardiac muscle, similar to |
| 2295 | 15041811 | Hermansky-Pudlak syndrome type-3 protein |
| 2296 | 15076827 | Pcph proto-oncogene protein |
| 2297 | 15079348 | angiotensinogen proteinase inhibitor, |
| 2298 | 15079392 | replication control protein 1 |
| 2299 | 15079408 | Unknown |
| 2300 | 15079735 | Unknown |
| 2301 | 15080291 | dipeptidyl peptidase 7+F206, similar to |
| 2302 | 15080429 | Unknown |
| 2303 | 15080454 | Unknown |
| 2304 | 15080499 | serineproteinase inhibitor, clade A (alpha-1 antiproteinase, antitrypsin), |
| | | member 1, similar to |
| 2305 | 15126735 | heat shock 27 kD protein 1, similar to |
| 2306 | 15147248 | putative breast epithelial stromal interaction protein |
| 2307 | 15147337 | progestin induced protein; ubiquitin-protein ligase [Homo sa |
| 2308 | 15149476 | arginyl-tRNA synthetase |
| 2309 | 15150811 | mitochondrial ribosomal protein S36 |
| 2310 | 15208648 | central cannabinoid receptor, isoform b; CB1 receptor; brain cannabinoid |
| | | receptor 1 |
| 2311 | 15213479 | putative DNA polymerase delta p38 subunit |
| 2312 | 15213542 | NSD1 |
| 2313 | 15214423 | Unknown |
| 2314 | 15214486 | Unknown |
| 2315 | 15214706 | Unknown |
| 2316 | 15215308 | dystroglycan 1, similar to |
| 2317 | 15227456 | ch-TOG protein from Homo sapiens [Arabidopsis tha |
| 2318 | 15277229 | Homologue to Drosophila photoreceptor protein calphotin |
| 2319 | 15277415 | scavenger receptor cysteine-rich type 1 protein M160 precursor |
| 2320 | 15277514 | Unknown |
| 2321 | 15278188 | Unknown |
| 2322 | 15281150 | unkempt (Drosophila)-like |
| 2323 | 15281837 | PX domain-containing protein kinase |
| 2324 | 15294558 | RAS-RELATED PROTEIN RAB-5A |
| 2325 | 15294560 | RAB5A, member RAS oncogene family |
| 2326 | 15294667 | bassoon (presynaptic cytomatrix protein) |
| 2327 | 15294817 | GalNAc-4-sulfotransferase 2 (H. sapiens), similar to |
| 2328 | 15295270 | MADhomolog 5 |
| 2329 | 15295351 | VDAC-1 |
| 2330 | 15295412 | Unknown |
| 2331 | 15295574 | laminin receptor1 |
| 2332 | 15295842 | Unknown |
| 2333 | 15296104 | optic atrophy 1 |
| 2334 | 15296351 | splicing factor 3b, subunit 1, 155 kD |
| 2335 | 15296762 | v-kit Hardy-Zuckerman 4 feline sarcoma viral oncogene homolog |
| 2336 | 15296824 | lipin 1 |
| 2337 | 15297926 | transforming growth factor, alpha |
| 2338 | 15298022 | mitochondrial ribosomal protein L53 |
| 2339 | 15299136 | Unknown |
| 2340 | 15299287 | Unknown |
| 2341 | 15299581 | Unknown |
| 2342 | 15299784 | glutamate receptor, metabotropic 1 |
| 2343 | 15299942 | Unknown |
| 2344 | 15300149 | modulator of transcription factor GATA-4 in cardiomyocytes |
| 2345 | 15301488 | SERINE/THREONINE PROTEIN KINASE 24(MST-3) |
| 2346 | 15302083 | CD2-associated protein |
| 2347 | 15302719 | Unknown |
| 2348 | 15302936 | citrate synthase precursor |
| 2349 | 15303880 | Glutamate receptor interacting protein |
| 2350 | 15304843 | Unknown |
| 2351 | 15304935 | destrin (actin depolymerizing factor) |
| 2352 | 15305404 | Unknown |
| 2353 | 15305472 | troponin I, cardiac |
| 2354 | 15305838 | ReIA-associated inhibitor |
| 2355 | 15306072 | transcriptional repressor NAC1 |
| 2356 | 15306753 | Unknown |
| 2357 | 15307117 | rho guanine nucleotide exchange factor 12 |
| 2358 | 15307634 | ND 23 k |
| 2359 | 15314651 | oxygen regulated protein |
| 2360 | 15318843 | aconitase 2, mitochondrial |
| 2361 | 15318933 | cytochrome b5 reductase |
| 2362 | 15321298 | Unknown |
| 2363 | 15321380 | v-erb-a avian erythroblastic leukemia viral oncogene homolog-like 4 |
| 2364 | 15321446 | Unknown |
| 2365 | 15341707 | Unknown |
| 2366 | 15375094 | RSK-like protein |
| 2367 | 15451842 | ADAM-TS disintegrin and metalloproteinase domain 19, isoform 1 |
| | | preproprotein; meltrin beta; metalloprotease-disintegrin meltrin beta |
| 2368 | 15451854 | midline 1, isoform beta; midline-1; zinc finger X and Y |
| 2369 | 15451916 | bone morphogenetic protein receptor, type II, isoform 1 precursor; type II |
| | | activin receptor-like kinase; serine/threonine kinase |
| 2370 | 15451923 | serologically defined colon cancer antigen 33 |
| 2371 | 15529996 | son of sevenless homolog 1 (Drosophila); son of sevenless (Drosophila) |
| | | homolog 1 |
| 2372 | 15530243 | villin 2 (ezrin), similar to |
| 2373 | 15530305 | Unknown |
| 2374 | 15553127 | hexokinase 2; hexokinase-2, muscle |
| 2375 | 15553137 | H2A-Bbd |
| 2376 | 15559225 | Unknown |
| 2377 | 15559303 | Unknown |
| 2378 | 15559516 | Unknown |
| 2379 | 15559753 | Unknown |
| 2380 | 15620821 | Unknown |
| 2381 | 15620841 | Unknown |
| 2382 | 15620853 | Unknown |
| 2383 | 15620867 | Unknown |
| 2384 | 15620879 | Unknown |
| 2385 | 15620927 | Unknown |
| 2386 | 15620933 | Unknown |
| 2387 | 15680004 | H2B histone family, member Q, similar to |
| 2388 | 15680171 | semaF cytoplasmic domain associated protein 3 |
| 2389 | 15718530 | POM121 membrane glycoprotein (rat homolog)-like 2 |
| 2390 | 15778991 | Unknown |
| 2391 | 15779080 | Unknown |
| 2392 | 15779126 | guanine nucleotide binding protein (G protein), a |
| 2393 | 15779156 | Unknown |
| 2394 | 15795410 | Unknown |
| 2395 | 15808373 | erythroid membrane-associated protein |
| 2396 | 15808607 | ATPase f F0 |
| 2397 | 15826629 | Peroxiredoxin 5 |
| 2398 | 15928608 | solute carrier family 25 (mitochondrial carrier; adenine nucleotide |
| | | translocator), member 5, similar to |
| 2399 | 15928907 | Unknown |
| 2400 | 15929030 | Unknown |
| 2401 | 15929352 | mitochondrial ribosomal protein L1 |
| 2402 | 15929856 | Unknown |
| 2403 | 15929892 | Unknown |
| 2404 | 15988268 | Myb-Domain Of Human Rap1 |
| 2405 | 15988350 | Lysozyme |
| 2406 | 15990494 | Unknown |
| 2407 | 15991827 | hexokinase 1, isoform HKI-R; brain form |
| 2408 | 15991829 | hexokinase 1, isoform HKI-ta/tb; brain form hexokinase |
| 2409 | 15991859 | Unknown |
| 2410 | 16033591 | SH2 domain-containing phosphatase anchor protein 2b |
| 2411 | 16041807 | Unknown |
| 2412 | 16156815 | Sec23-interacting protein p125 |
| 2413 | 16156952 | Unknown |
| 2414 | 16157047 | succinate dehydrogenase complex, subunit A, flavoprotein precursor |
| 2415 | 16157111 | progesterone membrane binding protein |
| 2416 | 16157253 | uridine 5 monophosphate hydrolase 1; pyrimidine 5-nucleotidase, similar |
| | | to |
| 2417 | 16157453 | Unknown |
| 2418 | 16157682 | IDN3 protein |
| 2419 | 16158005 | RNA-binding protein regulatory subunit |
| 2420 | 16158038 | putative, similar to |
| 2421 | 16158324 | heat shock 70 kD protein (Mortalin-2) |
| 2422 | 16158747 | CLIP-associating protein 2 |
| 2423 | 16159170 | Unknown |
| 2424 | 16159302 | Unknown |
| 2425 | 16159416 | Unknown |
| 2426 | 16159569 | Unknown |
| 2427 | 16159594 | carnitine palmitoyltransferase II |
| 2428 | 16159701 | ribosomal protein S7 (H. sapiens), similar to |
| 2429 | 16159788 | S100 calcium-binding protein A6 |
| 2430 | 16159874 | Unknown |
| 2431 | 16160276 | spectrin, beta, non-erythrocytic 1 (H. sapiens), similar to |
| 2432 | 16160441 | putative, similar to |
| 2433 | 16160793 | glycosyltransferase AD-017 |
| 2434 | 16160823 | phosphatidylinositol-4-phosphate 5-kinase, type I, beta |
| 2435 | 16160929 | retinoblastoma-binding protein 5 |
| 2436 | 16161569 | ryanodine receptor 2 |
| 2437 | 16161583 | endoplasmic reticulum oxidoreductin 1-Lbeta |
| 2438 | 16161627 | Rho guanine nucleotide exchange factor 10 |
| 2439 | 16161681 | Unknown |
| 2440 | 16161727 | stromal cell derived factor receptor 1 isoform a |
| 2441 | 16162032 | PEPTIDYL-PROLYL CIS-TRANS ISOMERASE B PRECURSOR |
| | | (PPIASE) (ROTAMASE) (CYCLOPHILIN B) |
| 2442 | 16163057 | Unknown |
| 2443 | 16163065 | RIKEN cDNA 2410008H17 gene, similar to |
| 2444 | 16163124 | TTF-I interacting peptide 20 |
| 2445 | 16163817 | Bcl 1 |
| 2446 | 16164710 | Unknown |
| 2447 | 16164895 | rabaptin-5 |
| 2448 | 16164980 | Unknown |
| 2449 | 16165190 | Unknown |
| 2450 | 16165554 | Unknown |
| 2451 | 16165872 | accessory proteins BAP31/BAP29 (H. sapiens), similar to |
| 2452 | 16166325 | Unknown |
| 2453 | 16166513 | pericentrin B |
| 2454 | 16168619 | Unknown |
| 2455 | 16171486 | Unknown |
| 2456 | 16171987 | monoamine oxidase A |
| 2457 | 16172349 | triadin |
| 2458 | 16174655 | Unknown |
| 2459 | 16175846 | atrophin-1 interacting protein 1; activin receptor interacting protein |
| 2460 | 16176937 | excision repair protein 1 |
| 2461 | 16177368 | putative, similar to |
| 2462 | 16177559 | MLL2 protein |
| 2463 | 16178062 | Unknown |
| 2464 | 16178117 | Unknown |
| 2465 | 16178214 | GTP-rho binding protein 1, similar to |
| 2466 | 16181084 | G protein-coupled receptor 51 |
| 2467 | 16192638 | isocitrate dehydrogenase 2 (NADP+), mitochondrial |
| 2468 | 16196598 | cox 6a |
| 2469 | 16198361 | Unknown |
| 2470 | 16198481 | Unknown |
| 2471 | 16306537 | cadherin 20, type 2 preproprotein |
| 2472 | 16306954 | Unknown |
| 2473 | 16306978 | annexin A2 |
| 2474 | 16307164 | CGI-90 protein |
| 2475 | 16307227 | Unknown |
| 2476 | 16307270 | Unknown |
| 2477 | 16307468 | Unknown |
| 2478 | 16307475 | neuroepithelial cell transforming gene 1 |
| 2479 | 16359102 | Unknown |
| 2480 | 16359195 | Unknown |
| 2481 | 16416451 | tRNA-nucleotidyltransferase |
| 2482 | 16418373 | Unknown |
| 2483 | 16418423 | guanylate binding protein 4 |
| 2484 | 16507813 | tumor necrosis factor receptor superfamily, member 21, similar to |
| 2485 | 16549125 | Unknown |
| 2486 | 16549199 | Unknown |
| 2487 | 16549271 | Unknown |
| 2488 | 16549294 | Unknown |
| 2489 | 16549620 | Unknown |
| 2490 | 16549880 | Unknown |
| 2491 | 16549918 | Unknown |
| 2492 | 16550394 | Unknown |
| 2493 | 16550518 | Unknown |
| 2494 | 16550576 | Unknown |
| 2495 | 16550810 | Unknown |
| 2496 | 16550845 | Unknown |
| 2497 | 16551173 | Unknown |
| 2498 | 16551429 | Unknown |
| 2499 | 16551580 | Unknown |
| 2500 | 16551610 | Unknown |
| 2501 | 16551739 | myosin light chain kinase |
| 2502 | 16551769 | Unknown |
| 2503 | 16551917 | Unknown |
| 2504 | 16551953 | Unknown |
| 2505 | 16551957 | Unknown |
| 2506 | 16552104 | Unknown |
| 2507 | 16552271 | Unknown |
| 2508 | 16552547 | Unknown |
| 2509 | 16552885 | Unknown |
| 2510 | 16552927 | Unknown |
| 2511 | 16552957 | Unknown |
| 2512 | 16552988 | Unknown |
| 2513 | 16553031 | Unknown |
| 2514 | 16553078 | Unknown |
| 2515 | 16553235 | Unknown |
| 2516 | 16553285 | Unknown |
| 2517 | 16553362 | Unknown |
| 2518 | 16554014 | Unknown |
| 2519 | 16554275 | Unknown |
| 2520 | 16554604 | mitochondrial ribosomal protein S23 |
| 2521 | 16554607 | mitochondrial ribosomal protein S10; NB4 apoptosis/differentiation related |
| | | protein; mitochondrial 28S ribosomal protein S10 |
| 2522 | 16741033 | protease 26S subunit, ATPase 1 |
| 2523 | 16753264 | Unknown |
| 2524 | 16876860 | Unknown |
| 2525 | 16877071 | ATPase gamma F1 |
| 2526 | 16877127 | synaptophysin-like protein, similar to |
| 2527 | 16877285 | duodenal cytochrome b, similar to |
| 2528 | 16877328 | Unknown |
| 2529 | 16877328 | Unknown |
| 2530 | 16877459 | Unknown |
| 2531 | 16877964 | isovaleryl Coenzyme A dehydrogenase |
| 2532 | 16878101 | Unknown |
| 2533 | 16924265 | Unknown |
| 2534 | 16924269 | Unknown |
| 2535 | 16950603 | mitochondrial ribosomal protein S35; mitochondrial 28S ribosomal protein |
| | | S28 |
| 2536 | 16950609 | mitochondrial ribosomal protein S27; mitochondrial 28S ribosomal protein |
| | | S27 |
| 2537 | 16974753 | sodium-potassium-chloride cotransporter |
| 2538 | 17016315 | olfactory receptor-like protein JCG4 |
| 2539 | 17028367 | gelsolin (amyloidosis, Finnish type), similar to |
| 2540 | 17028379 | Unknown |
| 2541 | 17375734 | Cyclin G-associated kinase |
| 2542 | 17378599 | Gamma-interferon-inducible protein Ifi-16 (Interferon-inducible myeloid |
| | | differentiation transcriptional activator) (IFI 16) |
| 2543 | 17380287 | Mitochondrial 39S ribosomal protein L56 (MRP-L56) (Serine beta |
| | | lactamase-like protein LACTB) |
| 2544 | 17380426 | Mannosyl-oligosaccharide 1,2-alpha-mannosidase IA (Processing alpha- |
| | | 1,2-mannosidase IA) (Alpha-1,2-mannosidase IA) (Mannosidase alpha |
| | | class 1A member 1) (Man(9)-alpha-mannosidase) (Man9-mannosidase) |
| 2545 | 17389971 | Unknown |
| 2546 | 17402865 | thiosulfate sulfurtransferase (rhodanese) |
| 2547 | 17432231 | MSTP022 |
| 2548 | 17434094 | putative, similar to |
| 2549 | 17434314 | Unknown |
| 2550 | 17434411 | Unknown |
| 2551 | 17434458 | Unknown |
| 2552 | 17434554 | Unknown |
| 2553 | 17434671 | Unknown |
| 2554 | 17435264 | INNER EAR-SPECIFIC COLLAGEN PRECURSOR (SACCULAR |
| | | COLLAGEN), similar to |
| 2555 | 17435299 | Unknown |
| 2556 | 17435748 | phosphorylase, glycogen; brain |
| 2557 | 17436258 | ND 13 K-B NADH dehydrogenase (ubiquinone) 1 alpha subcomplex, 5; |
| | | hypothetical protein FLJ12147; Complex I-13 KD-B; ubiquinone reductase; |
| | | type I dehydrogenase, similar to |
| 2558 | 17436498 | Unknown |
| 2559 | 17436513 | VDAC-1 VOLTAGE-DEPENDENT ANION-SELECTIVE CHANNEL |
| | | PROTEIN 1 (VDAC-1) (RVDAC1) (OUTER MITOCHONDRIAL |
| | | MEMBRANE PROTEIN PORIN 1), similar to |
| 2560 | 17436561 | Unknown |
| 2561 | 17436979 | Unknown |
| 2562 | 17437312 | Unknown |
| 2563 | 17438284 | Unknown |
| 2564 | 17439551 | REGULATOR OF G-PROTEIN SIGNALING 12 (RGS12), similar to |
| 2565 | 17440287 | anaplastic lymphoma kinase Ki-1, similar to |
| 2566 | 17442134 | one twenty two protein; hypothetical protein FLJ12479, similar to |
| 2567 | 17442500 | Molybdenum cofactor synthesis protein cinnamon, similar to |
| 2568 | 17442568 | Unknown |
| 2569 | 17443010 | hematological and neurological expressed sequence 1, similar to |
| 2570 | 17443439 | Unknown |
| 2571 | 17443833 | glyceraldehyde-3-phosphate dehydrogenase, similar to |
| 2572 | 17444067 | RIKEN cDNA 0610011N22, similar to |
| 2573 | 17444600 | Unknown |
| 2574 | 17444969 | solute carrier family 4, anion exchanger, member 3 |
| 2575 | 17445877 | xylulokinase homolog (H. influenzae) |
| 2576 | 17446038 | Unknown |
| 2577 | 17446807 | plastin 1 |
| 2578 | 17447126 | Unknown |
| 2579 | 17447383 | Unknown |
| 2580 | 17447877 | Unknown |
| 2581 | 17450039 | Unknown |
| 2582 | 17450491 | factor V, similar to |
| 2583 | 17451676 | putative, similar to |
| 2584 | 17451748 | Unknown |
| 2585 | 17451801 | Unknown |
| 2586 | 17452377 | Unknown |
| 2587 | 17454350 | putative protein, similar to |
| 2588 | 17454582 | phosphoglycerate mutase 1 (brain); Phosphoglycerate mutase A, |
| | | nonmuscle form, similar to |
| 2589 | 17455099 | putative, similar to |
| 2590 | 17455439 | heat shock 60 kD protein 1 (chaperonin) (H. sapiens), similar to |
| 2591 | 17455445 | Mitochondrial Complex I protein, now 21754001 |
| 2592 | 17455927 | Unknown |
| 2593 | 17456092 | Unknown |
| 2594 | 17456384 | non-specific cross reacting antigen, similar to |
| 2595 | 17457389 | Unknown |
| 2596 | 17458483 | Unknown |
| 2597 | 17458911 | Unknown |
| 2598 | 17459115 | Melanoma-associated antigen 11 (MAGE-11 antigen), similar to |
| 2599 | 17459319 | putative, similar to |
| 2600 | 17459408 | small Rho-like GTPase RhoA, similar to |
| 2601 | 17459479 | Unknown |
| 2602 | 17459746 | VOLTAGE-DEPENDENT ANION-SELECTIVE CHANNEL PROTEIN 2 |
| | | (OUTER MITOCHONDRIAL MEMBRANE PROTEIN PORIN 2), similar to |
| 2603 | 17460020 | Unknown |
| 2604 | 17460330 | Unknown |
| 2605 | 17460767 | Unknown |
| 2606 | 17460836 | testis expressed sequence 13A, similar to |
| 2607 | 17461025 | Unknown |
| 2608 | 17461670 | RIKEN cDNA 9430083G14, similar to |
| 2609 | 17462761 | Unknown |
| 2610 | 17463437 | Unknown |
| 2611 | 17464527 | match: multiple proteins; match: Q08151 P28185 Q01111 Q43554; |
| | | match: Q08150 Q40195 P20340 Q39222; match: Q40368 P36412 |
| | | P40393 Q40723; match: CE01798 Q38923 Q40191 Q41022; match: |
| | | Q39433 Q40177 Q40218 Q08146; match: P10949 P11023 Q, similar to |
| 2612 | 17464573 | Unknown |
| 2613 | 17464724 | eukaryotic translation elongation factor 1 alpha 1, similar to |
| 2614 | 17464807 | phosphoglycerate mutase 2 (muscle) |
| 2615 | 17464864 | Unknown |
| 2616 | 17465135 | v-raf murine sarcoma viral oncogene homolog B1 |
| 2617 | 17465213 | Unknown |
| 2618 | 17465562 | Unknown |
| 2619 | 17466365 | Unknown |
| 2620 | 17466818 | Unknown |
| 2621 | 17468096 | prohibitin, similar to |
| 2622 | 17468798 | Unknown |
| 2623 | 17469624 | Unknown |
| 2624 | 17470256 | Unknown |
| 2625 | 17470269 | chromosome 15 open reading frame 2, similar to |
| 2626 | 17470290 | Unknown |
| 2627 | 17471316 | Unknown |
| 2628 | 17471893 | Unknown |
| 2629 | 17472555 | Unknown |
| 2630 | 17472883 | ND 51K NADH dehydrogenase (ubiquinone) flavoprotein 1 (51 kD) |
| 2631 | 17474293 | midline 1; Finger on X and Y (in rat only on X), similar to |
| 2632 | 17474785 | VDAC-1 voltage-dependent anion channel 1, similar to |
| 2633 | 17475184 | Y39B6A.pp.p, similar to |
| 2634 | 17476245 | Unknown |
| 2635 | 17476469 | Unknown |
| 2636 | 17476471 | Unknown |
| 2637 | 17478738 | Unknown |
| 2638 | 17481443 | procollagen-proline, 2-oxoglutarate 4-dioxygenase (proline 4- |
| | | hydroxylase), beta polypeptide (protein disulfide isomerase; thyroid |
| | | hormone binding protein p55) |
| 2639 | 17481778 | Unknown |
| 2640 | 17482059 | Unknown |
| 2641 | 17482696 | Kruppel-type zinc finger (C2H2), similar to |
| 2642 | 17482910 | Unknown |
| 2643 | 17482953 | putative methyl-binding domain protein MBD105, similar to |
| 2644 | 17483121 | rhophilin-like protein (H. sapiens), similar to |
| 2645 | 17483187 | Unknown |
| 2646 | 17483399 | RAB11B, member RAS oncogene family |
| 2647 | 17483482 | Unknown |
| 2648 | 17484820 | acetyl-Coenzyme A synthetase 2 (AMP forming)-like |
| 2649 | 17484835 | Unknown |
| 2650 | 17485036 | Unknown |
| 2651 | 17485099 | Unknown |
| 2652 | 17485128 | Unknown |
| 2653 | 17485337 | Unknown |
| 2654 | 17485700 | Unknown |
| 2655 | 17485787 | Mitochondrial Acyl-CoA Thioesterase |
| 2656 | 17486071 | DKFZP434O047 protein, similar to |
| 2657 | 17486087 | Unknown |
| 2658 | 17486456 | Unknown |
| 2659 | 17486463 | Unknown |
| 2660 | 17486622 | Unknown |
| 2661 | 17486915 | Unknown |
| 2662 | 17487175 | dentin phosphoryn, similar to |
| 2663 | 17487390 | Unknown |
| 2664 | 17487672 | Unknown |
| 2665 | 17487733 | F40G9.9.p, similar to |
| 2666 | 17487809 | glyceraldehyde-3-phosphate dehydrogenase, similar to |
| 2667 | 17487981 | F4N2.10, similar to |
| 2668 | 17488153 | Unknown |
| 2669 | 17489631 | Unknown |
| 2670 | 17491107 | Unknown |
| 2671 | 17511874 | Unknown |
| 2672 | 17511976 | Unknown |
| 2673 | 17512080 | WAS protein family, member 1 |
| 2674 | 17512147 | Unknown |
| 2675 | 17736731 | mixed lineage kinase 4beta |
| 2676 | 17834080 | haymaker protein |
| 2677 | 17865554 | mitochondrial ribosomal protein L9, 60S mitochondrial precursor (L9 mt) |
| 2678 | 17939563 | Unknown |
| 2679 | 17943068 | Tcf-4 BETA-Catenin Complex |
| 2680 | 17943407 | Auh Protein, An Rna-Binding Homologue Of Enoyl-Coa Hydratase |
| 2681 | 17981863 | ND 5 |
| 2682 | 17985539 | ND 4 |
| 2683 | 18044194 | Unknown |
| 2684 | 18087815 | Unknown |
| 2685 | 18088572 | RIKEN cDNA 4930553C05 gene, similar to |
| 2686 | 18147097 | CG1800 gene product [Drosophila melanogaster] homolog |
| 2687 | 18157651 | bullous pemphigoid antigen 1 eA |
| 2688 | 18158416 | chromosome 20 open reading frame 188 protein; likely ortholog of mouse |
| | | transient receptor protein 4, associated protein |
| 2689 | 18201886 | chromosome 20 open reading frame 175 |
| 2690 | 18201913 | winged-helix nude |
| 2691 | 18204214 | Unknown |
| 2692 | 18204272 | Unknown |
| 2693 | 18252315 | propionyl-CoA carboxylase alpha subunit |
| 2694 | 18252778 | ankyrin repeat-containing protein ASB-2 |
| 2695 | 18490293 | ephrin B3, similar to |
| 2696 | 18490363 | calsequestrin 2 (cardiac muscle) |
| 2697 | 18490389 | Unknown |
| 2698 | 18490639 | Unknown |
| 2699 | 18543654 | Unknown |
| 2700 | 18543672 | Unknown |
| 2701 | 18544062 | Unknown |
| 2702 | 18544103 | transcription factor Dp-1, similar to |
| 2703 | 18544502 | Unknown |
| 2704 | 18545149 | SWI/SNF related, matrix associated, actin dependent regulator of |
| | | chromatin, subfamily f, member 1 (H. sapiens), similar to |
| 2705 | 18545197 | Unknown |
| 2706 | 18545286 | Unknown |
| 2707 | 18545525 | Unknown |
| 2708 | 18545711 | trithorax-related, similar to |
| 2709 | 18545867 | forkhead box D2 |
| 2710 | 18546369 | Unknown |
| 2711 | 18546495 | N-acetylglucosaminyltransferase VI, similar to |
| 2712 | 18547145 | Unknown |
| 2713 | 18547604 | Unknown |
| 2714 | 18547655 | Unknown |
| 2715 | 18547774 | PAPIN, similar to |
| 2716 | 18547995 | Unknown |
| 2717 | 18548319 | Unknown |
| 2718 | 18548686 | Unknown |
| 2719 | 18548841 | Unknown |
| 2720 | 18549011 | Unknown |
| 2721 | 18549603 | Unknown |
| 2722 | 18549721 | spectrin, alpha, erythrocytic 1 (elliptocytosis 2) |
| 2723 | 18549759 | Unknown |
| 2724 | 18550245 | Unknown |
| 2725 | 18550248 | dysferlin |
| 2726 | 18550356 | Unknown |
| 2727 | 18550688 | LWamide neuropeptide precursor protein, similar to |
| 2728 | 18551342 | laminin receptor 1; Laminin receptor-1 (67 kD); 67 kD, ribosomal protein |
| | | SA, similar to |
| 2729 | 18551404 | Unknown |
| 2730 | 18551428 | Unknown |
| 2731 | 18551530 | Unknown |
| 2732 | 18551750 | Unknown |
| 2733 | 18552428 | down-regulated by Ctnnb1, a, similar to |
| 2734 | 18552574 | heat shock 70 kD protein 9B (mortalin-2) (H. sapiens), similar to |
| 2735 | 18552843 | Unknown |
| 2736 | 18553054 | Unknown |
| 2737 | 18553524 | Unknown |
| 2738 | 18553646 | Unknown |
| 2739 | 18553709 | RIKEN cDNA 1810055D05 gene, similar to |
| 2740 | 18553922 | succinate dehydrogenase complex, subunit A, flavoprotein (Fp) (H. |
| | | sapiens) similar to |
| 2741 | 18554092 | Unknown |
| 2742 | 18554792 | Unknown |
| 2743 | 18554892 | protein phosphatase 4 regulatory subunit 2 (H. sapiens), similar to |
| 2744 | 18555498 | Unknown |
| 2745 | 18555697 | SALL1 (sal (Drosophila)-like, similar to |
| 2746 | 18555923 | Unknown |
| 2747 | 18556527 | protein tyrosine phosphatase, receptor type, G |
| 2748 | 18557013 | Unknown |
| 2749 | 18557341 | Unknown |
| 2750 | 18557515 | ring finger protein 23; RING-B box-coiled coil-B30.2, similar to |
| 2751 | 18557535 | Unknown |
| 2752 | 18557606 | Unknown |
| 2753 | 18557689 | Unknown |
| 2754 | 18558040 | Unknown |
| 2755 | 18558112 | C-terminal binding protein 1 (H. sapiens), similar to |
| 2756 | 18558130 | cyclin G associated kinase (H. sapiens), similar to |
| 2757 | 18558177 | Unknown |
| 2758 | 18558348 | Unknown |
| 2759 | 18558362 | Unknown |
| 2760 | 18558762 | Unknown |
| 2761 | 18559050 | Unknown |
| 2762 | 18559054 | Unknown |
| 2763 | 18559169 | GrpE-like protein cochaperone |
| 2764 | 18559889 | Unknown |
| 2765 | 18559896 | Unknown |
| 2766 | 18559969 | Unknown |
| 2767 | 18559997 | Unknown |
| 2768 | 18560088 | Unknown |
| 2769 | 18560396 | Unknown |
| 2770 | 18560536 | Unknown |
| 2771 | 18560871 | Unknown |
| 2772 | 18560910 | SGC32445 protein |
| 2773 | 18561153 | Unknown |
| 2774 | 18561225 | Unknown |
| 2775 | 18561342 | Unknown |
| 2776 | 18561850 | Unknown |
| 2777 | 18562164 | Unknown |
| 2778 | 18562264 | Unknown |
| 2779 | 18562403 | gag, similar to |
| 2780 | 18562447 | Unknown |
| 2781 | 18562613 | Unknown |
| 2782 | 18562676 | Unknown |
| 2783 | 18562743 | Unknown |
| 2784 | 18562778 | Unknown |
| 2785 | 18562814 | Unknown |
| 2786 | 18562826 | Unknown |
| 2787 | 18563024 | Unknown |
| 2788 | 18563079 | Unknown |
| 2789 | 18563446 | Unknown |
| 2790 | 18564249 | Unknown |
| 2791 | 18565200 | Unknown |
| 2792 | 18565553 | Unknown |
| 2793 | 18565735 | Unknown |
| 2794 | 18565792 | Unknown |
| 2795 | 18565965 | Unknown |
| 2796 | 18566008 | Unknown |
| 2797 | 18566051 | Unknown |
| 2798 | 18566469 | CDC14 cell division cycle 14 homolog B (S. cerevisiae) (H. sapiens), |
| | | similar to |
| 2799 | 18566582 | Unknown |
| 2800 | 18567546 | Unknown |
| 2801 | 18568015 | Unknown |
| 2802 | 18568092 | Unknown |
| 2803 | 18568100 | Unknown |
| 2804 | 18568732 | Unknown |
| 2805 | 18568834 | Unknown |
| 2806 | 18568892 | T-COMPLEX PROTEIN 1, GAMMA SUBUNIT (TCP-1-GAMMA) (CCT- |
| | | GAMMA), similar to |
| 2807 | 18568988 | Unknown |
| 2808 | 18569016 | Unknown |
| 2809 | 18569389 | Unknown |
| 2810 | 18569391 | Unknown |
| 2811 | 18569544 | Unknown |
| 2812 | 18569728 | Unknown |
| 2813 | 18569926 | Unknown |
| 2814 | 18570016 | Unknown |
| 2815 | 18570037 | Unknown |
| 2816 | 18571373 | Unknown |
| 2817 | 18571864 | Unknown |
| 2818 | 18572080 | tubulin, beta polypeptide 4, member Q (H. sapiens), similar to |
| 2819 | 18572219 | Unknown |
| 2820 | 18572532 | Unknown |
| 2821 | 18572576 | DKFZP434J193 protein (H. sapiens), similar to |
| 2822 | 18572752 | Unknown |
| 2823 | 18573432 | Unknown |
| 2824 | 18573604 | Unknown |
| 2825 | 18573884 | Sec24-related protein C |
| 2826 | 18574091 | (H. sapiens), similar to |
| 2827 | 18574564 | Unknown |
| 2828 | 18574897 | cathepsin L, similar to |
| 2829 | 18575014 | Unknown |
| 2830 | 18575020 | Unknown |
| 2831 | 18575034 | Unknown |
| 2832 | 18575353 | Unknown |
| 2833 | 18575792 | Unknown |
| 2834 | 18575881 | solute carrier family 9 (sodium/hydrogen exchanger), isoform 3, similar to |
| 2835 | 18575937 | Unknown |
| 2836 | 18576372 | Unknown |
| 2837 | 18576435 | glycoprotein beta-Gal 3′-sulfotransferase (H. sapiens), similar to |
| 2838 | 18576618 | Unknown |
| 2839 | 18576708 | Unknown |
| 2840 | 18576758 | Unknown |
| 2841 | 18576861 | Unknown |
| 2842 | 18577160 | Unknown |
| 2843 | 18577199 | suppression of tumorigenicity 5 |
| 2844 | 18577427 | Unknown |
| 2845 | 18577553 | Unknown |
| 2846 | 18577877 | glutamate receptor, metabotropic 5 (H. sapiens), similar to |
| 2847 | 18578024 | Unknown |
| 2848 | 18578981 | voltage gated potassium channel Kv3.2b, similar to |
| 2849 | 18579037 | glyceraldehyde-3-phosphate dehydrogenase, similar to |
| 2850 | 18579791 | Unknown |
| 2851 | 18580015 | Unknown |
| 2852 | 18580073 | Unknown |
| 2853 | 18580116 | solute carrier family 4, sodium bicarbonate cotransporter, member 8 (H. |
| | | sapiens), similar to |
| 2854 | 18580149 | Unknown |
| 2855 | 18580193 | Unknown |
| 2856 | 18580223 | Unknown |
| 2857 | 18580396 | Unknown |
| 2858 | 18580585 | Unknown |
| 2859 | 18580633 | phosphoinositide-3-kinase, class 2, gamma polypeptide |
| 2860 | 18581005 | Unknown |
| 2861 | 18581215 | Unknown |
| 2862 | 18581598 | Unknown |
| 2863 | 18581873 | Unknown |
| 2864 | 18582200 | Unknown |
| 2865 | 18582274 | Unknown |
| 2866 | 18582343 | Unknown |
| 2867 | 18582592 | Unknown |
| 2868 | 18582682 | CG9109 gene product, similar to |
| 2869 | 18582865 | Unknown |
| 2870 | 18583213 | Unknown |
| 2871 | 18583325 | Unknown |
| 2872 | 18583345 | Unknown |
| 2873 | 18583383 | Unknown |
| 2874 | 18583657 | Unknown |
| 2875 | 18583725 | multidomain presynaptic cytomatrix protein Piccolo, similar to |
| 2876 | 18583727 | Unknown |
| 2877 | 18584065 | Unknown |
| 2878 | 18584949 | Unknown |
| 2879 | 18585335 | Unknown |
| 2880 | 18585686 | Unknown |
| 2881 | 18586054 | Unknown |
| 2882 | 18586298 | Unknown |
| 2883 | 18586333 | splicing factor 3b, subunit 3, 130 kD |
| 2884 | 18586459 | putative, similar to |
| 2885 | 18586610 | Unknown |
| 2886 | 18587004 | Unknown |
| 2887 | 18587044 | Unknown |
| 2888 | 18587067 | Unknown |
| 2889 | 18587111 | Unknown |
| 2890 | 18587387 | Unknown |
| 2891 | 18587810 | arachidonate 12-lipoxygenase, 12R type (H. sapiens), similar to |
| 2892 | 18588235 | Unknown |
| 2893 | 18588450 | Unknown |
| 2894 | 18588517 | Unknown |
| 2895 | 18589035 | Unknown |
| 2896 | 18589065 | WW domain binding protein-2, similar to |
| 2897 | 18589260 | Unknown |
| 2898 | 18589408 | Unknown |
| 2899 | 18589876 | Unknown |
| 2900 | 18590023 | Unknown |
| 2901 | 18590390 | RNI-like protein, similar to |
| 2902 | 18590417 | Unknown |
| 2903 | 18590816 | Unknown |
| 2904 | 18591174 | Unknown |
| 2905 | 18591441 | ND B14.5a NADH dehydrogenase (ubiquinone) 1 alpha subcomplex, 7 |
| | | (14.5 kD, B14.5a) |
| 2906 | 18591813 | Unknown |
| 2907 | 18592023 | Unknown |
| 2908 | 18592069 | Unknown |
| 2909 | 18592852 | Unknown |
| 2910 | 18593545 | Unknown |
| 2911 | 18593908 | Unknown |
| 2912 | 18593939 | secretory protein 45 kDa, similar to |
| 2913 | 18594017 | Unknown |
| 2914 | 18594189 | Unknown |
| 2915 | 18594359 | Unknown |
| 2916 | 18594592 | Unknown |
| 2917 | 18594594 | Unknown |
| 2918 | 18594767 | Unknown |
| 2919 | 18594954 | Unknown |
| 2920 | 18594992 | Unknown |
| 2921 | 18595043 | Unknown |
| 2922 | 18595057 | Unknown |
| 2923 | 18595318 | Unknown |
| 2924 | 18595340 | Unknown |
| 2925 | 18595665 | Unknown |
| 2926 | 18596319 | glycerol kinase (H. sapiens), similar to |
| 2927 | 18596413 | Unknown |
| 2928 | 18596484 | Unknown |
| 2929 | 18596861 | RAS-RELATED PROTEIN RAB-15, similar to |
| 2930 | 18597225 | Unknown |
| 2931 | 18597549 | ZINC FINGER PROTEIN 268 (ZINC FINGER PROTEIN HZF3), similar to |
| 2932 | 18597551 | Unknown |
| 2933 | 18597742 | Unknown |
| 2934 | 18598132 | Unknown |
| 2935 | 18598291 | kinesin family member C3 |
| 2936 | 18598462 | Unknown |
| 2937 | 18598482 | Unknown |
| 2938 | 18598674 | Unknown |
| 2939 | 18598989 | Unknown |
| 2940 | 18599137 | zinc finger protein 2 (A1-5) |
| 2941 | 18599227 | Unknown |
| 2942 | 18599297 | EphB1 |
| 2943 | 18599533 | polyhomeotic 2 protein, similar to |
| 2944 | 18599587 | Unknown |
| 2945 | 18600174 | Unknown |
| 2946 | 18600186 | Unknown |
| 2947 | 18600274 | Unknown |
| 2948 | 18600320 | Unknown |
| 2949 | 18600459 | axonal transport of synaptic vesicles |
| 2950 | 18600477 | Unknown |
| 2951 | 18600510 | Unknown |
| 2952 | 18600673 | replication initiation region protein (60 kD) (H. sapiens), similar to |
| 2953 | 18600792 | Unknown |
| 2954 | 18600878 | Unknown |
| 2955 | 18600890 | Unknown |
| 2956 | 18601250 | Unknown |
| 2957 | 18601419 | Unknown |
| 2958 | 18601439 | Unknown |
| 2959 | 18601460 | Unknown |
| 2960 | 18601629 | huntingtin interacting protein-1-related (H. sapiens), similar to |
| 2961 | 18601927 | Unknown |
| 2962 | 18602066 | Unknown |
| 2963 | 18602347 | Unknown |
| 2964 | 18602382 | chromosome condensation-related SMC-associated protein 1 |
| 2965 | 18602858 | PUTATIVE NUCLEOSIDE DIPHOSPHATE KINASE (NDK) (NDP |
| | | KINASE), similar to |
| 2966 | 18602966 | Unknown |
| 2967 | 18603033 | Unknown |
| 2968 | 18603423 | Unknown |
| 2969 | 18603588 | solute carrier family 1 (glial high affinity glutamate transporter), member 2 |
| 2970 | 18603701 | Unknown |
| 2971 | 18603711 | Unknown |
| 2972 | 18603795 | Unknown |
| 2973 | 18603941 | PHOSPHATIDYLINOSITOL 3-KINASE REGULATORY SUBUNIT (IB PI3- |
| | | KINASE P101 SUBUNIT) (PTDINS-3-KINASE P101) (PI3K) (P101-PI3K), |
| | | similar to |
| 2974 | 18604379 | Unknown |
| 2975 | 18604520 | Unknown |
| 2976 | 18604537 | rab-related GTP-binding protein |
| 2977 | 18604876 | exostoses (multiple) 2 (H. sapiens), similar to |
| 2978 | 18605074 | Unknown |
| 2979 | 18605322 | Unknown |
| 2980 | 18605359 | Unknown |
| 2981 | 18606573 | Unknown |
| 2982 | 18645167 | annexin A2 |
| 2983 | 18676544 | Unknown |
| 2984 | 18676570 | Unknown |
| 2985 | 18676847 | Unknown |
| 2986 | 18860829 | optic atrophy 1, isoform 1 |
| 2987 | 18860843 | optic atrophy 1, isoform 7 |
| 2988 | 18916767 | Unknown |
| 2989 | 18916841 | Unknown |
| 2990 | 18959202 | leucine-rich PPR-motif containing; leucine-rich protein mRNA |
| 2991 | 19115954 | dynein, axonemal, heavy polypeptide 5 |
| 2992 | 19263915 | Unknown |
| 2993 | 19353103 | Unknown |
| 2994 | 19526647 | oxidored-nitro domain-containing protein |
| 2995 | 19584385 | Unknown |
| 2996 | 19684029 | Unknown |
| 2997 | 19743821 | integrin beta 1 isoform 1C-2 precursor; integrin VLA-4 beta subunit; |
| | | fibronectin receptor beta subunit |
| 2998 | 19850567 | breast carcinoma amplified sequence 3 |
| 2999 | 19923102 | holocarboxylase synthetase (biotin-[proprionyl-Coenzyme A-carboxylase |
| | | (ATP-hydrolysing)] ligase); Holocarbyoxylase synthetase; |
| | | holocarboxylase synthetase |
| 3000 | 19923233 | sterol carrier protein 2 |
| 3001 | 19923611 | Unknown |
| 3002 | 19923717 | rhysin 2 |
| 3003 | 19923721 | pre-T-cell receptor alpha precursor |
| 3004 | 19923757 | golgi autoantigen, golgin subfamily a, 2; golgin-95 |
| 3005 | 20070212 | voltage-dependent anion channel 3 |
| 3006 | 20070798 | androgen-regulated short-chain dehydrogenase/reductase 1 |
| 3007 | 20127408 | hydroxyacyl-Coenzyme A dehydrogenase/3-ketoacyl-Coenzyme A |
| | | thiolase/enoyl-Coenzyme A hydratase (trifunctional protein), alpha |
| | | subunit; Hydroxyacyl-Coenzyme A dehydrogenase/3-ketoacyl-Coenzyme |
| | | A thiolase/ |
| 3008 | 20127473 | glucose regulated protein, 58 kD |
| 3009 | 20127510 | peroxisomal long-chain acyl-coA thioesterase; peroxisomal long-chain |
| | | acyl-coA thioesterase; putative protein |
| 3010 | 20140018 | mitochondrial ribosomal protein S9, precursor (MRP-S9) |
| 3011 | 20140250 | Sideroflexin 1 |
| 3012 | 20141424 | Short chain 3-hydroxyacyl-CoA dehydrogenase, mitochondrial precursor |
| | | (HCDH) |
| 3013 | 20141538 | Homeobox protein Hox-C12 (Hox-3F) |
| 3014 | 20141568 | Isocitrate dehydrogenase [NADP], mitochondrial precursor |
| | | (Oxalosuccinate decarboxylase) (IDH) (NADP+-specific ICDH) (IDP) |
| | | (ICD-M) |
| 3015 | 20141580 | Mitochondrial 2-oxoglutarate/malate carrier protein (OGCP) |
| 3016 | 20141765 | Succinyl-CoA ligase [GDP-forming] alpha-chain, mitochondrial precursor |
| | | (Succinyl-CoA synthetase, alpha chain) (SCS-alpha) |
| 3017 | 20141946 | DNA topoisomerase II, beta isozyme |
| 3018 | 20147036 | transient receptor potential cation channel protein |
| 3019 | 20150348 | Deoxy Hbalphayq, A Mutant Of Hba |
| 3020 | 20151189 | Glutamate Dehydrogenase-Apo Form |
| 3021 | 20178093 | Suppressor of cytokine signaling 7 (SOCS-7) (Nck, Ash and |
| | | phospholipase C gamma-binding protein) (Nck-associated protein 4) |
| | | (NAP-4) |
| 3022 | 20268814 | CD36 antigen (collagen type I receptor, thrombospondin receptor) |
| 3023 | 20270305 | synaptotagmin-like 5 |
| 3024 | 20270399 | polycystic kidney and hepatic disease 1 |
| 3025 | 226207 | dihydrolipoamide S-acetyltransferase |

Table 2 presents a selected subset of the 3025 human heart mitochondrial proteins that are disclosed in Table 1 and in the Sequence Listing. The mitochondrial proteins of Table 2 are organized according to particular mitochondrial function classifications as indicated, based on analysis of amino acid sequences and GENBANK annotations; a number of the entries in Table 2 may use earlier GENBANK Accession numbers which differ from those shown in Table 1, but the sequences of such GENBANK Accession numbers can each be matched to a sequence in the Sequence Listing of the instant application using sequence database searching software tools as exemplified above and as known to the art (e.g., Basic Local Alignment Search Tool (“BLAST”), http://www.ncbi.nim.nih.gov/BLAST, Altschul, J. Mol. Biol. 219:555-565, 1991, Henikoff et al., Proc. Natl. Acad. Sci. USA 89:10915-10919, 1992; PSI-BLAST, ALIGN, MEGALIGN; WISETOOLS. CLUSTAL W, Thompson et al., 1994 Nucl. Ac. Res. 22:4673; CAP, www.no.embnet. org/clustalw.html; FASTA/FASTP, Pearson, 1990 Proc. Nat. Acad. Sci. USA 85:2444, available from D. Hudson, Univ. of Virginia, Charlottesville, Va.). As described above, each amino acid sequence provides a polypeptide structure from which a sample can be analyzed to determine, on the basis of structure, whether a modified polypeptide as provided herein may be present in the sample. As also described above, each functional classification refers to a defined biological activity measureable according to methods provided herein and known to the art, such that the invention contemplates determination in a sample of whether a polypeptide that exhibits altered biological activity is present.

TABLE 2
MITOCHONDRIAL FUNCTIONS OF SELECTED
COMPONENTS OF THE HUMAN HEART
MITOCHONDRIAL PROTEOME

| MITOCHONDRIAL FUNCTION | GENBANK | SEQ ID |
|---|---|---|
| CLASSIFICATION | ACC NO. | NO: |
| Amino acid metabolism | 118533 | 351 |
| | 2695812 | 563 |
| | 4504067 | 75 |
| | 4758714 | 527 |
| | 6624122 | 4 |
| | 11545863 | 520 |
| | 12653507 | 76 |
| | 13027640 | 491 |
| | 13518228 | 519 |
| | 14764412 | 240 |
| | 14775546 | 506 |
| | 16877964 | 453 |
| Amino acid metabolism Total | 12 | |
| Apoptosis | 2286145 | 159 |
| | 10437144 | 843 |
| | 10835173 | 637 |
| | 12382773 | 158 |
| | 14729475 | 101 |
| | 14761496 | 717 |
| | 16163817 | 100 |
| Apoptosis Total | 7 | |
| C-compound metabolism | 1354222 | 40 |
| | 4758498 | 405 |
| | 11275986 | 360 |
| | 11428230 | 37 |
| | 11436533 | 36 |
| | 12230075 | 359 |
| | 12652981 | 361 |
| | 13630862 | 39 |
| | 14043187 | 38 |
| | 14724751 | 695 |
| C-compound metabolism Total | 10 | |
| Carrier | 113463 | 153 |
| | 4505775 | 157 |
| | 4557403 | 155 |
| | 7657347 | 532 |
| | 11141885 | 851 |
| | 12232421 | 920 |
| | 12653827 | 531 |
| | 13632616 | 152 |
| | 13647558 | 151 |
| | 14747216 | 154 |
| | 14752024 | 850 |
| | 14774525 | 156 |
| Carrier Total | 12 | |
| Complex 1 | 13013 | 599 |
| | 1262579 | 583 |
| | 1262580 | 592 |
| | 4505355 | 620 |
| | 4505357 | 609 |
| | 4505359 | 613 |
| | 4505361 | 611 |
| | 4505365 | 617 |
| | 4505367 | 605 |
| | 4689104 | 610 |
| | 4758768 | 600 |
| | 4758772 | 621 |
| | 4758776 | 607 |
| | 4758784 | 614 |
| | 4758786 | 601 |
| | 4758790 | 588 |
| | 4758792 | 586 |
| | 4826848 | 612 |
| | 4826852 | 608 |
| | 4894370 | 619 |
| | 6041669 | 616 |
| | 7657369 | 591 |
| | 10092657 | 585 |
| | 10179599 | 622 |
| | 10764847 | 618 |
| | 10835025 | 596 |
| | 10835087 | 584 |
| | 12005918 | 369 |
| | 13097156 | 598 |
| | 13272567 | 602 |
| | 13272568 | 604 |
| | 13528960 | 590 |
| | 13637608 | 606 |
| | 14336775 | 623 |
| | 14769051 | 615 |
| | 14777313 | 587 |
| | 15307634 | 595 |
| Complex 1 Total | 37 | |
| Complex 2 | 4759080 | 865 |
| | 13639114 | 792 |
| | 14727486 | 867 |
| | 16157047 | 791 |
| Complex 2 Total | 4 | |
| Complex 3 | 117759 | 944 |
| | 117863 | 947 |
| | 190804 | 946 |
| | 1351360 | 934 |
| | 9297078 | 933 |
| | 11128019 | 233 |
| | 13631678 | 945 |
| | 13649658 | 948 |
| | 14736223 | 942 |
| | 14775827 | 943 |
| Complex 3 Total | 10 | |
| Complex 4 | 117103 | 211 |
| | 226209 | 221 |
| | 1262581 | 207 |
| | 4502985 | 213 |
| | 4502987 | 218 |
| | 4502989 | 217 |
| | 4502991 | 219 |
| | 4502993 | 220 |
| | 4758038 | 210 |
| | 4758040 | 215 |
| | 13629150 | 209 |
| | 13637833 | 216 |
| | 13648426 | 237 |
| | 16196598 | 212 |
| Complex 4 Total | 14 | |
| Complex 5 | 114549 | 84 |
| | 1262582 | 80 |
| | 4502297 | 87 |
| | 4502303 | 93 |
| | 5901896 | 89 |
| | 6005717 | 88 |
| | 11526149 | 85 |
| | 13272855 | 81 |
| | 13543618 | 83 |
| | 14774139 | 91 |
| Complex 5 Total | 10 | |
| DNA synthesis | 118749 | 497 |
| | 1709123 | 281 |
| | 4153874 | 840 |
| | 11225260 | 283 |
| DNA synthesis Total | 4 | |
| Glycolysis | 31645 | 355 |
| | 107554 | 752 |
| | 129070 | 750 |
| | 136066 | 921 |
| | 387011 | 751 |
| | 4557032 | 467 |
| | 11430299 | 401 |
| | 12653371 | 684 |
| | 13436413 | 350 |
| | 14043654 | 831 |
| | 14761208 | 356 |
| | 15553127 | 403 |
| | 15991827 | 402 |
| Glycolysis Total | 13 | |
| Guanine-related | 106185 | 372 |
| | 121009 | 379 |
| | 386745 | 380 |
| | 1335250 | 784 |
| | 4504049 | 378 |
| | 4506517 | 764 |
| | 6005772 | 747 |
| | 10047118 | 344 |
| | 10945428 | 516 |
| | 11055998 | 376 |
| | 14745808 | 377 |
| | 15779126 | 375 |
| | 16181084 | 343 |
| Guanine-related Total | 13 | |
| Inositol-related | 108480 | 688 |
| | 124505 | 433 |
| | 1399105 | 682 |
| | 4505801 | 686 |
| | 10835023 | 431 |
| | 11436778 | 435 |
| | 14724557 | 683 |
| | 14728229 | 687 |
| | 14760649 | 432 |
| | 14783738 | 434 |
| Inositol-related Total | 10 | |
| Kinase/phosphatase | 130749 | 45 |
| | 1103677 | 573 |
| | 1709242 | 650 |
| | 4503269 | 246 |
| | 4505153 | 510 |
| | 4506091 | 551 |
| | 4557769 | 522 |
| | 7439346 | 737 |
| | 10047120 | 437 |
| | 11526789 | 430 |
| | 12643716 | 738 |
| | 12654407 | 574 |
| | 12659007 | 733 |
| | 12830367 | 803 |
| | 13606056 | 280 |
| | 13631907 | 553 |
| | 13646385 | 222 |
| | 13648611 | 802 |
| | 13938619 | 224 |
| | 14194461 | 11 |
| | 14721507 | 801 |
| | 14733904 | 799 |
| | 14736227 | 774 |
| | 14740371 | 12 |
| | 14749765 | 10 |
| | 14782921 | 732 |
| | 14784064 | 552 |
| | 14785405 | 706 |
| | 15301488 | 418 |
| | 16033591 | 808 |
| Kinase/phosphatase Total | 30 | |
| Lipid metabolism | 1082723 | 722 |
| | 1169204 | 286 |
| | 1762533 | 148 |
| | 3273228 | 18 |
| | 4501869 | 22 |
| | 4502327 | 97 |
| | 4503607 | 295 |
| | 4503609 | 296 |
| | 4503651 | 322 |
| | 4504975 | 484 |
| | 4557817 | 869 |
| | 4557833 | 724 |
| | 4758312 | 297 |
| | 10835059 | 319 |
| | 11276083 | 323 |
| | 11433007 | 678 |
| | 11640566 | 421 |
| | 12669909 | 483 |
| | 12707570 | 304 |
| | 12805021 | 19 |
| | 13435350 | 327 |
| | 13639628 | 13 |
| | 13647276 | 465 |
| | 13653049 | 20 |
| | 14041699 | 310 |
| | 14043451 | 373 |
| | 14725848 | 21 |
| | 14729783 | 252 |
| | 14730775 | 420 |
| | 14746487 | 815 |
| | 14764159 | 14 |
| | 14764202 | 419 |
| | 14769776 | 674 |
| | 14781245 | 324 |
| Lipid metabolism Total | 34 | |
| Lipoprotein | 229479 | 480 |
| | 1082692 | 693 |
| | 4826914 | 691 |
| | 9438229 | 692 |
| | 13470094 | 70 |
| | 14721241 | 485 |
| Lipoprotein Total | 6 | |
| Nucleotide metabolism | 4502013 | 28 |
| | 4502457 | 78 |
| | 4503375 | 258 |
| | 8671846 | 204 |
| | 13654685 | 79 |
| | 14776778 | 77 |
| Nucleotide metabolism Total | 6 | |
| Protease | 4502201 | 30 |
| | 4502563 | 137 |
| | 7656959 | 139 |
| | 10047106 | 144 |
| | 12408656 | 136 |
| | 12643637 | 24 |
| | 12654627 | 517 |
| | 14772672 | 138 |
| | 14780055 | 727 |
| | 16741033 | 726 |
| Protease Total | 10 | |
| Protein targeting | 123571 | 385 |
| | 1091688 | 390 |
| | 1346317 | 387 |
| | 4008131 | 184 |
| | 5032181 | 915 |
| | 5802970 | 33 |
| | 6912714 | 916 |
| | 7657257 | 917 |
| | 7662673 | 918 |
| | 9910382 | 533 |
| | 12655195 | 391 |
| | 13645492 | 389 |
| | 14603309 | 386 |
| Protein targeting Total | 13 | |
| ras/GTPase | 1657266 | 789 |
| | 5803135 | 755 |
| | 11359874 | 371 |
| | 11436135 | 761 |
| | 12652715 | 648 |
| | 12751117 | 704 |
| | 13569962 | 845 |
| | 13651229 | 772 |
| | 13652324 | 760 |
| | 13786129 | 417 |
| | 13794267 | 757 |
| | 14211570 | 202 |
| | 14249144 | 754 |
| | 14740792 | 1390 |
| ras/GTPase Total | 14 | |
| Receptor | 184477 | 771 |
| | 1001941 | 257 |
| | 1168781 | 316 |
| | 4504733 | 436 |
| | 4877291 | 763 |
| | 11968152 | 852 |
| | 13632266 | 894 |
| | 13650874 | 748 |
| | 14732886 | 895 |
| | 14744234 | 646 |
| | 16161569 | 788 |
| Receptor Total | 11 | |
| Redox | 802150 | 662 |
| | 4502601 | 143 |
| | 4557845 | 775 |
| | 6912536 | 633 |
| | 11399466 | 239 |
| | 11416669 | 632 |
| | 12804319 | 142 |
| | 13112023 | 199 |
| | 13236495 | 753 |
| | 13529257 | 41 |
| | 13627233 | 42 |
| | 13994325 | 744 |
| | 14735899 | 235 |
| Redox Total | 13 | |
| Stress | 4503731 | 331 |
| | 4758192 | 800 |
| | 5453902 | 634 |
| | 7643782 | 383 |
| | 13631440 | 675 |
| | 14250063 | 676 |
| | 14755436 | 874 |
| Stress Total | 7 | |
| Structural | 13194197 | 459 |
| | 13643253 | 460 |
| | 14124976 | 461 |
| | 14730782 | 462 |
| | 15305472 | 924 |
| Structural Total | 5 | |
| TCA cycle | 417178 | 450 |
| | 1071834 | 256 |
| | 1170477 | 451 |
| | 1718502 | 16 |
| | 5031777 | 448 |
| | 5174539 | 500 |
| | 11321581 | 872 |
| | 11321583 | 868 |
| | 11374664 | 452 |
| | 12804901 | 449 |
| | 13627252 | 658 |
| | 13639817 | 505 |
| | 14740547 | 342 |
| | 14782063 | 501 |
| | 15318843 | 17 |
| | 16192638 | 446 |
| TCA cycle Total | 16 | |
| Transcription | 105294 | 48 |
| | 107912 | 905 |
| | 1033182 | 1400 |
| | 1582692 | 888 |
| | 2565032 | 904 |
| | 4506445 | 780 |
| | 4507389 | 301 |
| | 6678455 | 908 |
| | 6912440 | 287 |
| | 9884738 | 67 |
| | 11096171 | 783 |
| | 11761696 | 119 |
| | 11890755 | 782 |
| | 12653775 | 394 |
| | 12734816 | 741 |
| | 13242069 | 647 |
| | 13787197 | 242 |
| | 13938539 | 232 |
| | 14730158 | 889 |
| | 14742266 | 781 |
| | 14748858 | 910 |
| | 14766373 | 765 |
| | 14790190 | 847 |
| | 15296351 | 859 |
| | 15300149 | 558 |
| | 15451854 | 530 |
| | 16163124 | 926 |
| Transcription Total | 27 | |
| Translation | 1706611 | 300 |
| | 4503507 | 311 |
| | 4758118 | 243 |
| | 5032051 | 6 |
| | 7661872 | 474 |
| | 7705626 | 543 |
| | 7706349 | 546 |
| | 11177148 | 535 |
| | 11416393 | 538 |
| | 11424404 | 544 |
| | 11559927 | 542 |
| | 11596859 | 537 |
| | 13027604 | 547 |
| | 13123976 | 73 |
| | 13559404 | 534 |
| | 13631521 | 549 |
| | 13648964 | 35 |
| | 13899231 | 541 |
| | 14028389 | 539 |
| | 14028405 | 545 |
| | 14165270 | 536 |
| | 14285174 | 299 |
| | 15150811 | 548 |
| | 15295574 | 469 |
| | 15298022 | 540 |
| Translation Total | 25 | |
| Transport | 28714 | 52 |
| | 114374 | 579 |
| | 1172554 | 1394 |
| | 1359715 | 578 |
| | 1588292 | 130 |
| | 4503057 | 225 |
| | 5729937 | 518 |
| | 5730033 | 848 |
| | 7799988 | 470 |
| | 8923870 | 408 |
| | 10716563 | 135 |
| | 10835220 | 94 |
| | 11612670 | 690 |
| | 12803281 | 1395 |
| | 13376991 | 1396 |
| | 13540606 | 875 |
| | 13649217 | 1393 |
| | 14149607 | 186 |
| | 14739472 | 710 |
| | 14767738 | 134 |
| | 14778381 | 294 |
| | 16974753 | 849 |
| Transport Total | 22 | |
| Tumor-related | 120749 | 498 |
| | 132164 | 768 |
| | 1177438 | 123 |
| | 4507643 | 930 |
| | 10567164 | 348 |
| | 10835155 | 928 |
| | 10863907 | 397 |
| | 12246901 | 929 |
| | 12643796 | 770 |
| | 13529047 | 912 |
| | 13650639 | 515 |
| | 14725399 | 898 |
| | 14755336 | 931 |
| | 15076827 | 665 |
| | 15296762 | 1388 |
| | 16160929 | 769 |
| Tumor-related Total | 16 | |
| Zinc finger | 1177230 | 1401 |
| | 2117022 | 1402 |
| | 2317769 | 714 |
| | 3021386 | 1403 |
| | 4507979 | 1404 |
| | 4827065 | 1405 |
| | 5454180 | 1407 |
| | 7671629 | 464 |
| | 14211907 | 1410 |
| | 14286186 | 1406 |
| | 14670360 | 1409 |
| | 14755316 | 3025 |
| | 14755456 | 1408 |
| Zinc finger Total | 13 | |

### Example 4

### OXIDATIVE POST-TRANSLATIONAL MODIFICATION OF TRYPTOPHAN RESIDUES IN CARDIAC MITOCHONDRIAL PROTEINS

This example shows the distribution of N-formylkynurenine, a product of the dioxidation of tryptophan residues in proteins, throughout the human heart mitochondrial proteome. This oxidized amino acid was associated with a distinct subset of proteins, including an over-representation of complex I subunits as well as complex V subunits and enzymes involved in redox metabolism. No relationship was observed between the tryptophan modification and methionine oxidation, a known artifact of sample handling. As the mitochondria were isolated from normal human heart tissue and not subject to any artificially induced oxidative stress, the susceptible tryptophan residues in this group of proteins appeared, according to non-limiting theory, to be “hot spots” for oxidation in close proximity to a source of reactive oxygen species (ROS) in respiring mitochondria.

LC/MS/MS data generated from the human heart mitochondrial proteome project as described in the preceding Examples, as well as data for human and bovine proteins prepared by sucrose density gradient centrifugation as described above, or by immunoprecipitation using antibodies against complex V (ATP synthase) and/or complex I (NADH dehydrogenase) proteins (see, Table 2), were queried against the human or bovine subsets of GenBank using the Sonar MSMS searching algorithm (Genomic Solutions, Ann Arbor, Mich.) with oxidation of methionine (+16 u) and tryptophan (+32 u) specified as differential modifications. Corresponding MALDI spectra were manually inspected. FIG. 3 shows oxidation products of tryptophan from proteins, including N-formylkynurenine (Structure 2).

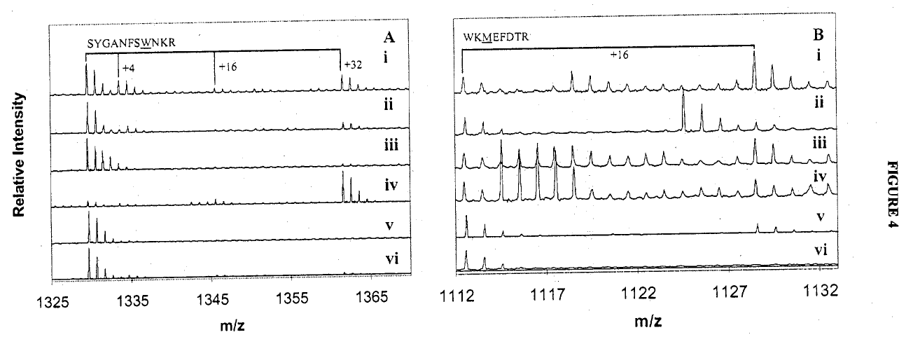

Modifications to complex I subunits in bovine heart mitochondria in response to the oxidative stress caused by peroxynitrite treatment were studied in vitro, and yielded evidence of oxidized tryptophan in several subunits, both by MALDI TOF and by LC/MS/MS. Surprisingly, the relative intensities of the peaks in the MALDI spectra corresponding to peptides containing N-formylkynurenine were also high in untreated mitochondria from some bovine and human heart preparations, although there was substantial variation. Prior to complex I isolation and electrophoresis, mitochondria were prepared identically from all hearts which were freshly collected, frozen and thawed immediately prior to analysis. FIG. 4 shows the MALDI spectra of peptides from the human complex I subunit, NDUFS4 (see Table 3), and its bovine homologue from five different preparations corresponding to seven different hearts (five human, including one pooled sample of mitochondria from three individual hearts, and two bovine hearts). The relative intensities of m/z 1329.6 and 1361.6 (corresponding to peptides without and with dioxidized tryptophan, FIG. 4A) and 1112.5 and 1128.5 (corresponding to peptides without and with oxidized methionine, FIG. 4B) were used as a rough measure of protein oxidation. No correlation was found between the extent of tryptophan oxidation and that of methionine oxidation, suggesting that they occurred via different mechanisms.

The dioxidation of tryptophan was clearly discernable in FIG. 4A (i) and (ii) in which complex I was purified by different methods, sucrose density gradient centrifugation or immunoprecipitation, respectively, but corresponded to mitochondria from the same human heart. This finding suggested that the method of preparation was not a factor in determining the extent of oxidation, but rather that such oxidation was a characteristic of the donor from which the sample was obtained (in this case, a 41-year-old male Caucasian who died of brain cancer). The other human donor, displaying far less extensive oxidation of tryptophan as seen in FIG. 4A (iii), was a 62-year-old female Caucasian who died of intracranial bleeding. In contrast, NDUFS4 from a pool of mitochondria from three human hearts displayed an extensively oxidized tryptophan-containing peptide FIG. 4A (iv). Again the degree of oxidation in the pooled sample was not commensurate with the degree of oxidation for the methionine-containing fragment FIG. 4B (iv).

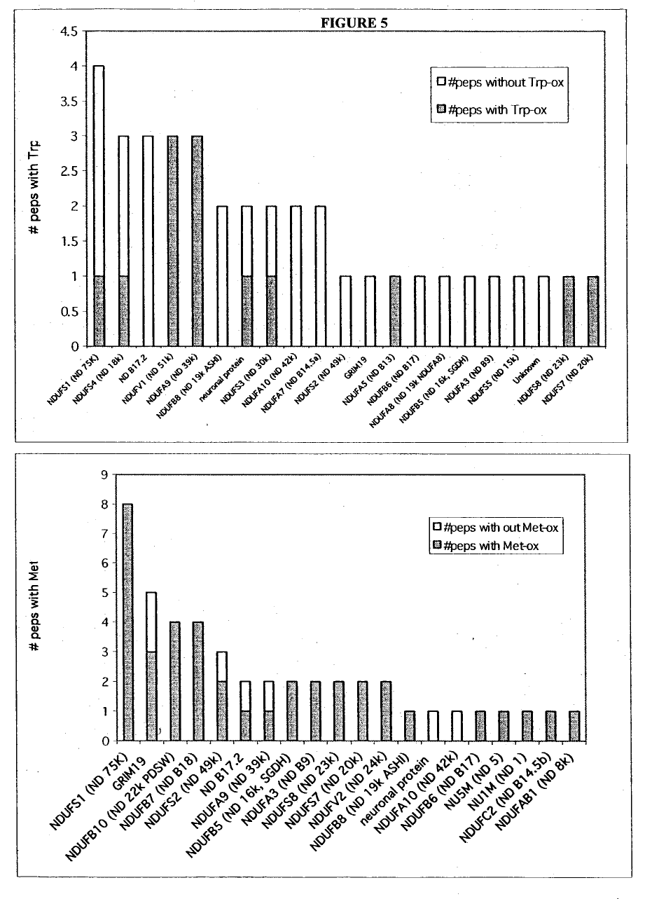

Distribution of the oxidatively modified tryptophan in the MS/MS spectra dataset described in the preceding Examples was assessed by reanalyzing the data with N-formylkynurenine selected as a differential modification of tryptophan (+32) using the SonarMSMS algorithm according to the supplier's instructions (Genomic Solutions, Ann Arbor, Mich.). Table 3 lists N-formylkynurenine-containing peptides found with peptide expect scores (Epep) values ≦1×10⁻² (99% confidence); also listed in Table 3 are the identifiers for the mitochondrial polypeptide sequences from which these peptides derived. Of this list of 51 peptide sequences from 39 proteins, 9 subunits of complex I had N-formylkyenurine-containing tryptic peptides and included two newly discovered subunits (Table 1, NCBI/Genbank Acc. Nos. 13938442 and 17455445, now 21754001). This subset of proteins was used to compare tryptophan oxidation versus methionine oxidation as a function of the ability to observe a peptide in any given LC/MS/MS experiment. As shown in FIG. 5, the numbers of distinct peptides containing methionine (A) and tryptophan (B) were plotted for a given complex I subunit which had a Sonar MSMS Epep score of ≦1×10⁻², and on each plot FIG. 5 indicates whether the corresponding oxidized residue was observed. Methionine oxidation appeared to be directly related to the number of observable peptides that would be expected if oxidation were a random sample-handling artifact. In contrast, tryptophan oxidation appeared to be much more specific to selected subunits, with the greatest modification being noted for NDUFV1 (51 kDa flavoprotein 1) and NDUFA9 (a 39 kDa reductase/isomerase subunit). In addition, five subunits of the iron-protein component were oxidized.

TABLE 3
PEPTIDES CONTAINING DOUBLY OXIDIZED
TRYPTOPHAN FROM THE CARDIAC
MITOCHONDRIAL PROTEOME.

| | | Peptide | |
|---|---|---|---|
| | | Derived | |
| | | from | |
| | | NCBI/ | |
| | | Genbank | PROTEIN |
| PEPTIDE | Epep | Acc. No. | DESCRIPTION |
| VFEISPFEPwITR | 1.40E−05 | 6681764 | NDUFA9 |
| FGPIPLGSLGwK | 2.30E−04 | 6681764 | NDUFA9 |
| wLSAEIEDVKPAK | 1.80E−03 | 6681764 | NDUFA9 |
| HAGGVTGGwDNLLAVIPGGS | 2.10E−04 | 20149568 | NDUFV1 |
| STPLIPK | | | |
| GDARPAEIDSLwEISK | 9.40E−04 | 20149568 | NDUFV1 |
| GPDwILGEIK | 2.40E−03 | 20149568 | NDUFV1 |
| LAALPENPPAIDwAYYK | 3.20E−05 | 5453559 | ATPase d F0 |
| TIDwVAFAEIIPQNQK | 2.10E−03 | 5453559 | ATPase d F0 |
| YPYwPHQPIENL | 7.20E−03 | 5453559 | ATPase d F0 |
| wVVIGDENYGEGSSR | 8.40E−08 | 3600098 | aconitase precursor |
| VAEKEGwPLDIR | 4.00E−04 | 3600098 | aconitase precursor |
| LwISNGGLADIFTVFAK | 2.90E−06 | 18044943 | acyl-Coenzyme A |
| | | | dehydrogenase, very long chain |
| IFGSEAAwK | 3.90E−03 | 18044943 | acyl-Coenzyme A |
| | | | dehydrogenase, very |
| | | | long chain |
| ALGVLAQLIwSR | 1.10E−05 | 4758076 | citrate synthase precursor |
| DYIwNTLNSGR | 7.10E−04 | 4758076 | citrate synthase |
| | | | precursor |
| KLETAVNLAwTAGNSNTR | 1.60E−05 | 4507879 | VDAC-1 |
| wNTDNTLGTEITVEDQLAR | 5.30E−03 | 4507879 | VDAC-1 |
| VVDGAVGAQwLAEFR | 4.70E−05 | 17458911 | dihydrolipoamide |
| | | | S-acetyltransferase |
| VPEANSSwMDTVIR | 6.60E−04 | 17458911 | dihydrolipoamide |
| | | | S-acetyltransferase |
| SAVTALwGK | 3.70E−03 | 4504349 | beta globin |
| LLVVYPwTQR | 4.30E−03 | 4504349 | beta-globin |
| RPPEPTTPwQEDPEPEDENL | 6.80E−08 | 13938442 | neuronal protein |
| YEK | | | (ND17.3) |
| NLTQYSwLLDGFPR | 1.00E−06 | 19923437 | adenylate kinase 3 |
| | | | alpha like |
| FDLNSPwEAFPVYR | 2.10E−05 | 11360206 | NDUFS3 |
| IASGLGLAwIVGR | 2.60E−05 | 4758714 | microsomal glutathione |
| | | | S-transferase 3 |
| GYIVIEDLwK | 2.90E−05 | 12001992 | brain my025 |
| ASSTSPVEISEwLDQK | 4.00E−05 | 4503607 | electron transfer |
| | | | flavoprotein alpha |
| | | | polypeptide |
| GRPTSTNPIASIFAwTR | 6.40E−05 | 4504575 | isocitrate |
| | | | dehydrogenase 2 |
| | | | (NADP+), |
| | | | mitochondrial |
| GLLTYTSwEDALSR | 1.40E−04 | 21411235 | NDUFS1 |
| IPwFQYPIIYDIR | 1.90E−04 | 6005854 | D-prohibitin |
| GLSDGEwQLVLNVwGK | 2.50E−04 | 229361 | Myoglobin |
| ASwSSLSMDEK | 3.00E−04 | 5921895 | Cytochrome c oxidase |
| | | | subunit IV isoform 1 |
| LDDLVNwAR | 5.30E−04 | 21750696 | NDUFS7 |
| TLLwTELFR | 7.80E−04 | 4505371 | NDUFS8 |
| SYGANFSwNK | 8.70E−04 | 13528960 | NDUFS4 |
| ASLHALVGSPIIwGGEPR | 9.90E−04 | 13676336 | long-chain acyl-coA |
| | | | thioesterase |
| | | | peroxisomal |
| wEVADLQPQLK | 1.20E−03 | 21903482 | Ubiquinol-cytochrome |
| | | | C reductase complex |
| | | | core protein 2 |
| YEGFFSLwK | 1.30E−03 | 21361114 | mitochondrial carrier; |
| | | | oxoglutarate carrier |
| LITTQQwLIK | 1.40E−03 | 13272660 | ATP synthase 6 |
| LWEPLVEEPPADQwK | 1.50E−03 | 4826848 | NDUFA5 |
| IDEAILITwTK | 2.00E−03 | 15991833 | hexokinase 1 |
| wDGQETTLVR | 3.30E−03 | 458862 | fatty acid binding |
| | | | protein, heart; hFABP |
| HwLDSPwPGFFTLDGQPR | 3.40E−03 | 20541592 | 2-oxoglutarate |
| | | | dehydroqenase E1 |
| | | | component, |
| | | | mitochondrial |
| | | | precursor |
| AwNGSAEGPGKVER | 4.30E−03 | 21754001 | Unnamed protein |
| | | | product (NDUFB11) |
| ELwFSDDPNVTK | 4.70E−03 | 4757732 | programmed cell death |
| | | | 8 (apoptosis-inducing |
| | | | factor AIF) |
| EQwDTIEELIR | 5.30E−03 | 4503301 | 2,4-dienoyl CoA |
| | | | reductase 1 precursor |
| GAwSNVLR | 5.30E−03 | 86754 | carrier ANT |
| wYYNAAGFNK | 5.30E−03 | 5454152 | UCR ubiquinone- |
| | | | binding protein (VI) |
| ELDSITPEVLPGwK | 5.50E−03 | 8131894 | Mitofilin |
| APLAEEwDNMTMK | 8.10E−03 | 4505093 | monoamine oxidase B |
| LATFwYYAK | 9.10E−03 | 22096328 | ATP synthase G chain, |
| | | | mitochondrial |

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying a mitochondrial target for therapeutic intervention in treatment of a disease associated with altered mitochondrial function, comprising:
(a) determining a presence, in a biological sample from a subject known to have or suspected of having a disease associated with altered mitochondrial function, of at least one modified polypeptide, said modified polypeptide comprising at least one modification to a polypeptide having an amino acid sequence as set forth in any one of SEQ ID NOS 1-3025; and
(b) correlating the modification with at least one disease associated with altered mitochondrial function, and therefrom identifying a mitochondrial target for therapeutic intervention.

2. The method of claim 1 wherein the modified polypeptide exhibits altered biological activity.

3. The method of claim 1 wherein the biological sample is selected from the group consisting of blood, skin, skeletal muscle, liver and cartilage.

4. The method of claim 1 wherein the disease associated with altered mitochondrial function is selected from the group consisting of Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF) and cancer.

5. The method of claim 1 wherein the modification is selected from the group consisting of an amino acid substitution, an amino acid insertion, an amino acid deletion, a posttranslational modification and an altered expression level.

6. The method of claim 4 wherein the posttranslational modification is selected from the group consisting of glycosylation, phosphorylation, nitration, nitrosylation, amidation, fatty acylation and oxidative modification.

7. A method of identifying an agent for treating a disease associated with altered mitochondrial function, comprising:
(a) contacting a candidate agent with a biological sample from a subject having a disease associated with altered mitochondrial function, wherein said sample comprises at least one polypeptide that exhibits altered biological activity which accompanies said disease and wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025; and
(b) determining an increase or decrease in the altered biological activity of the polypeptide in the presence of the candidate agent relative to the level of the altered biological activity in the absence of the candidate agent, and therefrom identifying an agent for treating a disease associated with altered mitochondrial function.

8. The method of claim 7 wherein the altered biological activity is an indicator of altered mitochondrial function that is selected from the group consisting of ATP biosynthesis, oxidative phosphorylation, calcium uptake, calcium release, maintenance of inner mitochondrial membrane potential, mitochondrial permeability transition, ETC-mediated electron transport and intermembrane space protein release.

9. The method of claim 7 wherein the sample is selected from the group consisting of a cell, a mitochondria enriched sample, an isolated mitochondrion and a submitochondrial particle.

10. The method of claim 7 wherein the disease associated with altered mitochondrial function is selected from the group consisting of Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF), and cancer.

11. A method of treating a disease associated with altered mitochondrial function comprising administering to a subject in need thereof an agent that compensates for at least one biological activity of a polypeptide that exhibits altered biological activity which accompanies said disease, wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025.

12. A method for identifying a risk for having or a presence of a disease associated with altered mitochondrial function, comprising:
(a) determining a presence, in a biological sample from a subject suspected of having a disease associated with altered mitochondrial function, of at least one modified polypeptide, said modified polypeptide comprising at least one modification to a polypeptide having an amino acid sequence as set forth in any one of SEQ ID NOS 1-3025, wherein the modification correlates with at least one disease associated with altered mitochondrial function, and therefrom identifying a risk for or presence of disease.

13. A method of identifying an agent for treating a disease associated with altered mitochondrial function, comprising:
(a) contacting a candidate agent with an isolated polypeptide that exhibits altered biological activity which accompanies a disease associated with altered mitochondrial function, wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025; and
(b) determining an increase or decrease in the altered biological activity of the polypeptide in the presence of the candidate agent relative to the level of the altered biological activity in the absence of the candidate agent, and therefrom identifying an agent for treating a disease associated with altered mitochondrial function.

14. The method of claim 13 wherein the disease associated with altered mitochondrial function is selected from the group consisting of Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fiber syndrome (MERRF), and cancer.

15. The method of claim 13 wherein the isolated polypeptide is present in a preparation that is selected from the group consisting of a submitochondrial particle, a proteoliposome and a mitochondrial protein fraction.

16. A method of identifying an agent for treating a disease associated with altered mitochondrial function, comprising:
(a) administering a candidate agent to a subject having a disease associated with altered mitochondrial function; and
(b) determining, in a first biological sample obtained from the subject prior to the step of administering the candidate agent and in a second biological sample obtained from the subject subsequent to the step of administering the candidate agent, wherein each of said first and second samples comprises at least one polypeptide that exhibits altered biological activity which accompanies said disease and wherein the polypeptide is selected from the group consisting of (i) a polypeptide having an amino acid sequence as set forth in any one of SEQ ID NOS 1-3025 and (ii) a modified polypeptide that comprises at least one modification to a polypeptide having an amino acid sequence as set forth in any one of SEQ ID NOS 1-3025,
an increase or decrease in the altered biological activity of the polypeptide in the second sample relative to the level of the altered biological activity in the first sample, and therefrom identifying an agent for treating a disease associated with altered mitochondrial function.

17. The method of claim 16 wherein the altered biological activity is an indicator of altered mitochondrial function that is selected from the group consisting of ATP biosynthesis, oxidative phosphorylation, calcium uptake, calcium release, maintenance of inner mitochondrial membrane potential, mitochondrial permeability transition, ETC-mediated electron transport and intermembrane space protein release.

18. The method of claim 16 wherein the sample is selected from the group consisting of a cell, a mitochondria enriched sample, an isolated mitochondrion and a submitochondrial particle.

19. The method of claim 16 wherein the disease associated with altered mitochondrial function is selected from the group consisting of Alzheimer's disease, diabetes mellitus, Parkinson's disease, Huntington's disease, osteoarthritis, dystonia, Leber's hereditary optic neuropathy (LHON), mitochondrial encephalopathy, lactic acidosis, and stroke (MELAS), myoclonic epilepsy ragged red fibersyndrome (MERRF), and cancer.
